(19) United States
(12) Patent Application Publication
(10) Pub. No.: US 2025/0191759 A1
(43) Pub. Date: Jun. 12, 2025

(54) Breast Cancer Diagnostic and Treatment

(71) Applicants: Adriana DE SIERVI, Ciudad Autónoma de Buenos Aires (AR); Consejo Nacional de Investigaciones Científicas y Técnicas (CONICET), Ciudad Autónoma de Buenos Aires (AR)

(72) Inventors: Paula Lucia FARRE, Ciudad Autónoma de Buenos Aires (AR); Rocio Belen DUCA, Ciudad Autónoma de Buenos Aires (AR); Adriana DE SIERVI, Ciudad Autónoma de Buenos Aires (AR)

(21) Appl. No.: 18/846,215

(22) PCT Filed: Mar. 10, 2023
(86) PCT No.: PCT/IB2023/052334

§ 371 Date: Sep. 11, 2024

Related U.S. Application Data
(60) Provisional application No. 63/319,188, filed on Mar. 11, 2022.

(51) Int. Cl. G16H 50/20; C12Q 1/6851; C12Q 1/6886; G06N 20/10; G16H 20/17; G16H 50/30; G16H 50/70

(52) U.S. Cl. CPC G16H 50/20; C12Q 1/6851; C12Q 1/6886; G06N 20/10; G16H 20/17; G16H 50/30; G16H 50/70; C12Q 2600/158; C12Q 2600/178

(57) ABSTRACT

The present disclosure related to methods of diagnosis and treatment of breast cancer comprising determining the expression level of at least four miRNAs selected from the group consisting of miR-150-5p, miR-106b-3p, miR-106a-5p, miR-125a-5p, miR-17-5p, miR-574-3p, miR-339-5p, miR-339-3p, miR-335-5p, miR-16-5p, and miR-21-5p. Selection of specific groups of patients and specific therapies can be made based on the patterns of expression observed for at least four of the disclosed biomarkers. The present disclosure also provides methods of enriching a sample obtained from a patient, diagnostic kits comprising oligonucleotides capable of hybridizing with the disclosed biomarkers.

### CROSS-REFERENCE TO RELATED APPLICATIONS AND INCORPORATION BY REFERENCE

This PCT application claims the priority benefit of U.S. Provisional Application No. 63/319,188, filed on Mar. 11, 2022, which is herein incorporated by reference in its entirety.

### REFERENCE TO SEQUENCE LISTING SUBMITTED ELECTRONICALLY

The content of the electronically submitted sequence listing (Name: 3181_009PC01_Seqlisting_ST26; Size: 42,909 bytes; and Date of Creation: Mar. 3, 2023) is herein incorporated by reference in its entirety.

### FIELD

The present disclosure related to methods and compositions for the diagnosis and treatment of breast cancer comprising the use of a panel of microRNA biomarkers.

### BACKGROUND

Breast cancer remains the second leading cause of cancer-related deaths in women. Although the discovery of BRCA1 and BRCA2 were important steps in identifying key genetic factors involved in breast cancer, it has become clear that mutations in BRCA1 and BRCA2 account for only a fraction of inherited susceptibility to breast cancer (Nathanson et al. (2001) Human Mol. Gen. 10(7):715-720; Anglican Breast Cancer Study Group (2000) Br. J. Cancer 83(10):1301-08; Syrjakoski et al. (2000) J. Natl. Cancer Inst. 92:1529-31). Despite considerable research into therapies for breast cancer, breast cancer remains difficult to diagnose and treat effectively, and the high mortality observed in breast cancer patients indicates that improvements are needed in the diagnosis, treatment and prevention of the disease.

Currently, the gold standard early detection method for breast cancer is mammography, which is complemented by other diagnostic imaging techniques such as magnified mammography, breast ultrasound and/or nuclear magnetic resonance. Finally, molecular analysis is performed from a standard biopsy. In turn, there are tumor markers in the blood such as CEA, CA125 and CA15-3 (Chen et al. (2020) Cancer research, 80(2), 170-174).

Regarding mammography, the sensitivity values of the technique, together with specificity and other metrics such as positive predictive value (PPV) or area under the curve (AUC) have been widely reported in various works, and systematic reviews and meta-analysis comparing different works. Various authors reported that they found sensitivity and specificity values for mammography of 52 and 90.5% (Sorin et al (2018) American Journal of Roentgenology, W267-W274), in other cases 81 and 96% (Li et al (2016) Radiology, 281(2), 382-391.6), 82 and 94% (Kim et al (2019) Korean Journal of Radiology, 20(2), 218-224), from 87% (without reporting specificity values) (Niell et al. (2017) Radiologic Clinics, 55(6), 1145-1162) to 97 and 64.5% (Zeeshan et al (2018) Cureus, 10(4)). This allows us to conclude that the sensitivity and specificity associated with mammography are highly variable, due to various factors that include the type of equipment and the personnel involved in the analysis. In turn, it has been seen that the sensitivity of mammography decreases drastically in cases of women with dense breasts (30-63% vs 76-98%), which makes it a problem in the early diagnosis of breast cancer in these women. (Niell et al. 2017).

Some miRNAs have been proposed as biomarkers for breast cancers (see, e.g., U.S. Pat. No. 8,148,069, CN105586401, US2020347457, WO2015035480, CN108004318, US2018230544, CN109609633, U.S. Ser. No. 10/316,367, U.S. Ser. No. 10/059,998, US2017175203, U.S. Pat. Nos. 7,955,848, 8,288,356, U.S. Ser. No. 10/526,602, US2013065778, WO2007140352, AU2013245505, US2012219958, and AU2016203583. miRNAs are small non-coding RNA molecules that can silence the gene expression of multiple genes. miRNAs are attractive for use as biomarkers because they can be released into the extracellular space, complexed with other molecules or packaged in exosomes, and circulate in body fluids such as blood.

Liquid biopsy is a material obtained from a peripheral blood sample of a patient in order to look for tumor cells or fragments of circulating tumor nucleic acid, such as DNA, RNA or non-coding RNA (such as miRNAs). Liquid biopsies can be used to detect cancer at an early stage, even when the tumor is undetectable by other diagnostic methods. It has been determined that the detection of miRNAs temporarily precedes the appearance of a tumor image, which could be an advantage when compared with images as an early detection method. They could also be used to predict the evolution of patients and/or establish a personalized therapeutic plan.

To date, clinical techniques to diagnose and treat breast cancer do not involve the use of miRNA biomarkers. The present disclosure provides novel combinations of miRNAs biomarkers that, combined with the use of machine learning, yield diagnostic tools that are capable of early detection of breast cancer with high sensitivity and specificity.

### BRIEF SUMMARY

The present disclosure provides a method for determining the breast cancer status in a subject in need thereof, comprising applying a machine-learning classifier to a plurality of miRNA expression levels obtained from a miRNA biomarker panel from a sample from the subject, wherein the machine-learning classifier identifies the subject as having or not having breast cancer. Also provided is method for treating a human subject afflicted with breast cancer comprising administering a breast cancer therapy to the subject, wherein, prior to the administration, the subject is identified as having or not having a specific breast cancer status determined by applying a machine-learning classifier to a plurality of miRNA expression levels obtained from a miRNA biomarker panel from a sample obtained from the subject. The present disclosure also provides a method for treating a human subject afflicted with breast cancer comprising (i) identifying, prior to the administration, a subject having or not having a specific breast cancer status by applying a machine-learning classifier to a plurality of miRNA expression levels obtained from a miRNA biomarker panel from a sample obtained from the subject; and, (ii) administering a breast cancer therapy to the subject. Also provide is a method for identifying a human subject afflicted with a breast cancer suitable for treatment with a breast cancer therapy, the method comprising applying a machine-learning classifier to a plurality of miRNA expression levels obtained from a miRNA biomarker panel from sample obtained from the subject, wherein the assignment of the sample to a specific breast cancer status, indicates that a specific breast cancer therapy can be administered to treat the cancer.

In some aspects, the machine-learning classifier is a model obtained by Linear Regression, Random Forest, Logistic Regression, Artificial Neural Network (ANN), Support Vector Machine (SVM), XGBoost (XGB), glmnet, cforest, Classification and Regression Trees for Machine-learning (CART), treebag, K-Nearest Neighbors (kNN), or a combination thereof. In some aspects, the Linear Regression is Lasso Regression.

In some aspects, the miRNA biomarker panel comprises miR-150-5p, miR-106b-3p, miR-106a-5p, miR-125a-5p, miR-17-5p, miR-574-3p, miR-339-5p, miR-339-3p, miR-335-5p, miR-16-5p and miR-21-5p. In some aspects, the miRNA biomarker panel consists of miR-150-5p, miR-106b-3p, miR-106a-5p, miR-125a-5p, miR-17-5p, miR-574-3p, miR-339-5p, miR-339-3p, miR-335-5p, miR-16-5p and miR-21-5p. In some aspects, the miRNA biomarker panel comprises at least four miRNAs selected from the group consisting of miR-150-5p, miR-106b-3p, miR-106a-5p, miR-125a-5p, miR-17-5p, miR-574-3p, miR-339-5p, miR-339-3p, miR-335-5p, miR-16-5p and miR-21-5p. In some aspects, the miRNA biomarker panel consists of at least five miRNAs selected from the group consisting of miR-150-5p, miR-106b-3p, miR-106a-5p, miR-125a-5p, miR-17-5p, miR-574-3p, miR-339-5p, miR-339-3p, miR-335-5p, miR-16-5p and miR-21-5p. In some aspects, the miRNA biomarker panel comprise 4, 5, 6, 7, 8, 9, 10, or 11 miRNAs selected from the group consisting of miR-150-5p, miR-106b-3p, miR-106a-5p, miR-125a-5p, miR-17-5p, miR-574-3p, miR-339-5p, miR-339-3p, miR-335-5p, miR-16-5p and miR-21-5p. In some aspects, the miRNA biomarker panel comprises miR-106a-5p, miR-17-5p, miR-339-3p, miR-16-5p, miR-150-5p, miR-125a-5p, miR-339-5p, and miR-335-5p. In some aspects, the miRNA biomarker panel comprises miR-106a-5p, miR-339-3p, miR-16-5p, miR-150-5p and miR-339-5p. In some aspects, the miRNA biomarker panel comprises miR-106a-5p, miR-17-5p, miR-339-3p, miR-16-5p and miR-335-5p. In some aspects, the miRNA biomarker panel comprises miR-106a-5p, miR-17-5p, miR-339-3p, and miR-16-5p.

In some aspects, the miRNA biomarker panel comprises miR-150-5p, miR-106b-3p, miR-106a-5p, miR-125a-5p, miR-17-5p, miR-574-3p, miR-339-5p, miR-335-5p, miR-16-5p and miR-21-5p. In some aspects, the miRNA biomarker panel comprises miR-106a-5p, miR-150-5p, miR-125a-5p, miR-17-5p, miR-339-5p, miR-335-5p and miR-16-5p. In some aspects, the miRNA biomarker panel consists of miR-150-5p, miR-106b-3p, miR-106a-5p, miR-125a-5p, miR-17-5p, miR-574-3p, miR-339-5p, miR-335-5p, miR-16-5p and miR-21-5p. In some aspects, the miRNA biomarker panel consists of miR-106a-5p, miR-150-5p, miR-125a-5p, miR-17-5p, miR-339-5p, miR-335-5p and miR-16-5p

In some aspects, the sample comprises blood. In some aspects, the blood is venous blood. In some aspects, the miRNA expression levels are determined using quantitative real-time PCR (qPCR), sequencing (miRNA-seq), miRNA expression microarrays, DNA biosensors, or any technology that measures RNA. In some aspects, the machine-learning classifier is trained with miRNA expression data obtained from a reference population.

The present disclosure also provides a classifier for determining the breast cancer status of sample from a subject in need thereof, wherein the classifier identifies the sample as having a specific breast cancer status using as input miRNA expression levels obtained from a miRNA biomarker panel comprises miR-150-5p, miR-106b-3p, miR-106a-5p, miR-125a-5p, miR-17-5p, miR-574-3p, miR-339-5p, miR-339-3p, miR-335-5p, miR-16-5p and miR-21-5p, or a subset thereof from a sample from the subject, and wherein the breast cancer status indicates that the subject can be effectively treated with a breast cancer therapy. In some aspects, the miRNA biomarker panel comprise 4, 5, 6, 7, 8, 9, 10, or 11 miRNAs selected from the group consisting of miR-150-5p, miR-106b-3p, miR-106a-5p, miR-125a-5p, miR-17-5p, miR-574-3p, miR-339-5p, miR-339-3p, miR-335-5p, miR-16-5p and miR-21-5p. In some aspects, the miRNA biomarker panel comprises miR-106a-5p, miR-17-5p, miR-339-3p, miR-16-5p, miR-150-5p, miR-125a-5p, miR-339-5p, and miR-335-5p. In some aspects, the miRNA biomarker panel comprises miR-106a-5p, miR-339-3p, miR-16-5p, miR-150-5p and miR-339-5p. In some aspects, the miRNA biomarker panel comprises miR-106a-5p, miR-17-5p, miR-339-3p, miR-16-5p and miR-335-5p. In some aspects, the miRNA biomarker panel comprises miR-106a-5p, miR-17-5p, miR-339-3p, and miR-16-5p. In some aspects, the sample comprises blood. In some aspects, the blood is venous blood.

In some aspects, the calculation of the breast cancer status comprises obtaining the probability according to a statistical model, wherein the statistical model is a logistic regression. In some aspects, the statistical model is cross validated with machine learning model. In some aspects, the machine-learning model is Cross Validation Leave One Out. In some aspects, the calculation of the breast cancer status comprises: (i) averaging the Ct values obtained from the qPCR for each of the miRNAs biomarkers; (ii) subtracting the average value of each of the miRNAs from the Ct value obtained from a control; (iii) squaring the subtracting result of the previous step; (iv) calculating the logarithm in base e for the result of the step iii, obtaining an individual value for each miRNA (Value X); (v) calculating the probability by integrating each result of the previous steps according to: p(x)=1/(e\hat{ }(−(βₒ+βₙ*Value X))+1), wherein the β is a specific coefficient related to the statistical model selected; (vi) comparing the obtained probability score (p(x)) with a cut-off, wherein if the value of the individual's probability of having breast cancer is equal to or greater than the cut-off point, the individual will be classified as sick, and if it is less than the cut-off point, it will be classified as healthy. In some aspects, the sample is enriched in at least one miRNA from the miRNA biomarker panel comprises miR-150-5p, miR-106b-3p, miR-106a-5p, miR-125a-5p, miR-17-5p, miR-574-3p, miR-339-5p, miR-339-3p, miR-335-5p, miR-16-5p and miR-21-5p.

The present disclosure provides a sample comprising body fluid enriched in at least one miRNA from the miRNA biomarker panel comprises miR-150-5p, miR-106b-3p, miR-106a-5p, miR-125a-5p, miR-17-5p, miR-574-3p, miR-339-5p, miR-339-3p, miR-335-5p, miR-16-5p and miR-21-5p. In some aspects, the body fluid is selected from the consisting of blood, plasma, serum, urine, saliva, lacrimal fluid, and fluids obtainable from the breast glands. In some aspects, the breast cancer treatment is based in a breast cancer therapy selected from the group consisting of chemotherapy, anti-hormone therapy, targeted therapy, immunotherapy, and any combination thereof.

In some aspects of the methods and classifiers disclosed above, the breast cancer status comprises absence or presence of breast cancer. In some aspects, the breast cancer is selected from the group consisting of: metastatic, and non-metastatic. In some aspects, the breast cancer status comprises a breast cancer risk score. In some aspects, the breast cancer status comprises a breast cancer prognosis or outcome score. In some aspects, the breast cancer status comprises a breast cancer response to a specific breast cancer therapy. In some aspects, the breast cancer status comprises a breast cancer stage score. In some aspects, the breast cancer stage is selected from the group consisting of: T, N, M and any combination thereof.

In some aspects, administering the breast cancer therapy reduces the cancer burden. In some aspects, cancer burden is reduced by at least about 10%, at least about 20%, at least about 30%, at least about 40%, or about 50% compared to the cancer burden prior to the administration.

In some aspects, the subject exhibits progression-free survival of at least about one month, at least about 2 months, at least about 3 months, at least about 4 months, at least about 5 months, at least about 6 months, at least about 7 months, at least about 8 months, at least about 9 months, at least about 10 months, at least about 11 months, at least about one year, at least about eighteen months, at least about two years, at least about three years, at least about four years, or at least about five years after the initial administration. In some aspects, the subject exhibits stable disease about one month, about 2 months, about 3 months, about 4 months, about 5 months, about 6 months, about 7 months, about 8 months, about 9 months, about 10 months, about 11 months, about one year, about eighteen months, about two years, about three years, about four years, or about five years after the initial administration. In some aspects, the subject exhibits a partial response about one month, about 2 months, about 3 months, about 4 months, about 5 months, about 6 months, about 7 months, about 8 months, about 9 months, about 10 months, about 11 months, about one year, about eighteen months, about two years, about three years, about four years, or about five years after the initial administration. In some aspects, the subject exhibits a complete response about one month, about 2 months, about 3 months, about 4 months, about 5 months, about 6 months, about 7 months, about 8 months, about 9 months, about 10 months, about 11 months, about one year, about eighteen months, about two years, about three years, about four years, or about five years after the initial administration.

In some aspects, the administering improves progression-free survival probability by at least about 10%, at least about 20%, at least about 30%, at least about 40%, at least about 50%, at least about 60%, at least about 70%, at least about 80%, at least about 90%, at least about 100%, at least about 110%, at least about 120%, at least about 130%, at least about 140%, or at least about 150%, compared to the progression-free survival probability of a subject not diagnosed using a classifier of the present disclosure. The term “classifier of the present disclosure” refers to a breast cancer classifier disclosed herein, e.g., PM1, PM2, PM3, PM4, PM5, a combination thereof, or a classification model generated as disclosed herein. In some aspects, the administering improves overall survival probability by at least about 25%, at least about 50%, at least about 75%, at least about 100%, at least about 125%, at least about 150%, at least about 175%, at least about 200%, at least about 225%, at least about 250%, at least about 275%, at least about 300%, at least about 325%, at least about 350%, or at least about 375%, compared to the overall survival probability of a subject not diagnosed using a classifier of the present disclosure.

The present disclosure provides a miRNA biomarker panel comprising miR-150-5p, miR-106b-3p, miR-106a-5p, miR-125a-5p, miR-17-5p, miR-574-3p, miR-339-5p, miR-339-3p, miR-335-5p, miR-16-5p and miR-21-5p, for use in determining the breast cancer status of a subject in need thereof using a machine-learning classifier of the present, wherein the breast cancer status is used for (i) identifying a subject suitable for an anticancer therapy; (ii) determining the prognosis of a subject undergoing anticancer therapy; (iii) initiating, suspending, or modifying the administration of an anticancer therapy; or, (iv) a combination thereof.

Also provided is a therapy for treating breast cancer in a human subject in need thereof, wherein the subject is identified as having a breast cancer status according to the machine-learning classifier of the present disclosure, wherein the breast cancer status makes the subject eligible for treatment with a breast cancer therapy selected from the group consisting of chemotherapy, anti-hormone therapy, targeted therapy, immunotherapy, or any combination thereof.

The present disclosure provides a method of assigning a breast cancer status to a subject in need thereof, the method comprising (i) generating a machine-learning model by training a machine-learning method with a training set comprising miRNA expression levels for each gene in a gene panel in a plurality of samples obtained from a plurality of subjects, wherein each sample is assigned a breast cancer status classification; and, (ii) assigning, using the machine-learning model, the breast cancer status to the subject, wherein the input to the machine-learning model comprises miRNA expression levels for each gene in the gene panel in a test sample obtained from the subject.

Also provided is a method of assigning a breast cancer status to a subject in need thereof, the method comprising generating a machine-learning model by training a machine-learning method with a training set comprising miRNA expression levels for each gene in a gene panel in a plurality of samples obtained from a plurality of subjects, wherein each sample is assigned a breast cancer status classification; wherein the machine-learning model assigns a breast cancer status to the subject using as input miRNA expression levels for each gene in the gene panel in a test sample obtained from the subject.

The present disclosure provides a method of assigning a breast cancer status to a subject in need thereof, the method comprising using a machine-learning model to predict the breast cancer status of the subject, wherein the machine-learning model is generated by training a machine-learning method with a training set comprising miRNA expression levels for each gene in a gene panel in a plurality of samples obtained from a plurality of subjects, wherein each sample is assigned a breast cancer status classification.

In some aspects, a classifier or method disclosed herein is implemented in a computer system comprising at least one processor and at least one memory, the at least one memory comprising instructions executed by the at least one processor to cause the at least one processor to implement the machine-learning model. In some aspects, the computer implemented method comprises (i) inputting, into the memory of the computer system, the machine-learning model; (ii) inputting, into the memory of the computer system, the miRNA biomarker panel input data corresponding to the subject, wherein the input data comprises miRNA expression levels; (iii) executing the machine-learning model; or, (v) any combination thereof.

The present disclosure also provides a kit for the detection of breast cancer, comprising: (i) specific oligonucleotides for reverse transcription of miR-16-5p, miR-17-5p, miR-106a-5p, and miR-339-3p in a sample; (ii) oligonucleotides for quantitative PCR of miR-16-5p, miR-17-5p, miR-106a-5p, miR-339-3p; and a universal oligonucleotide Rv. Also provided is a kit for the detection of breast cancer comprising: (i) specific oligonucleotides for reverse transcription of miR-106a-5p, miR-17-5p, miR-339-3p, miR-16-5p and miR-335-5p in a sample; (ii) oligonucleotides for quantitative PCR of miR-106a-5p, miR-17-5p, miR-339-3p, miR-16-5p, and miR-335-5p; and a universal oligonucleotide Rv. Also provided is a kit for the detection of breast cancer comprising: (i) specific oligonucleotides for reverse transcription of miR-106a-5p, miR-17-5p, miR-339-3p, miR-16-5p, miR-150-5p, miR-125a-5p, miR-339-5p, and miR-335-5p in a sample; (ii) oligonucleotides for quantitative PCR of miR-106a-5p, miR-17-5p, miR-339-3p, miR-16-5p, miR-150-5p, miR-125a-5p, miR-339-5p, and miR-335-5p; and a universal oligonucleotide Rv. Also provided is a kit for the detection of breast cancer comprising: (i) specific oligonucleotides for reverse transcription of miR-150-5p, miR-106b-3p, miR-106a-5p, miR-125a-5p, miR-17-5p, miR-574-3p, miR-339-5p, miR-339-3p, miR-335-5p, miR-16-5p, and miR-21-5p in a sample; (ii) oligonucleotides for quantitative PCR of miR-150-5p, miR-106b-3p, miR-106a-5p, miR-125a-5p, miR-17-5p, miR-574-3p, miR-339-5p, miR-339-3p, miR-335-5p, miR-16-5p and miR-21-5p and a universal oligonucleotide Rv. In some aspects, the kit further comprises specific oligonucleotides for the control, e.g., a cel39 control. In some aspects, the kit further comprises synthetic positive controls for the quantitative PCR step. In some aspects, the kit further comprises a procedures manual.

### BRIEF DESCRIPTION OF THE DRAWINGS/FIGURES

FIG. 1. Diagram of the samples and cohorts of volunteers enrolled in the clinical protocol. Plasma samples from patients with breast cancer and healthy donors (HD) from different hospitals were collected for the identification and validation of miRNAs that can be used in the diagnosis of breast cancer. Expression data from microarrays were also obtained from the serum of patients with breast cancer and HD from public repositories with which the external validation cohort was established. The number of samples in each case is indicated. ES=Early Stages; AS=Advanced Stages.

FIG. 2. Scheme of the selection of candidate biomarker miRNAs obtained from the different patient cohorts and technologies. Two technologies were used for the identification of candidate miRNAs, expression microarrays and miRNA sequencing. Plasma from patients with breast cancer or HD from the exploratory cohort was used for expression microarrays or six breast cancer patients and four HD from the validation cohort. Then, with the data obtained, comparisons of the lists of miRNAs obtained were made, with different selection criteria (p-val<0.05 or <0.2 and Fold-change >1.5 or >0 as appropriate. Finally, two final groups of miRNAs. The number of volunteers enrolled and miRNAs obtained is indicated in all cases.

FIGS. 3A, 3B and 3C. Validation of candidate miRNAs in the exploratory cohort. Expression of the indicated circulating miRNAs measured by RT-qPCR from plasma of patients with breast cancer (n=30) or HD (n=36). The data is normalized to the spike-in cel-miR-39-3p. Box plots are shown with dots indicating each sample individually to show the variability corresponding to each experimental group. The statistical significance used was 5% and the data were analyzed using the T-test, Wilcoxon test or the median test, as appropriate. The term n.s. was used when the differences were not significant.

FIGS. 4A, 4B and 4C. ROC curves corresponding to the miRNAs measured in the exploratory cohort. Using the expression data of the 11 candidate miRNAs measured by RT-qPCR from the plasma of patients with breast cancer (n=30) and HD (n=36), the corresponding ROC curves were made using logistic models. Area under the curve (AUC) values, their corresponding confidence intervals (CI) and associated p-values were calculated, which are shown in the table in the lower right corner. The statistical significance used was 5%.

FIGS. 5A, 5B and 5C. Validation of candidate miRNAs in the validation cohort. Expression of the indicated circulating miRNAs measured by RT-qPCR from plasma of patients with breast cancer (n=100) or HD (n=73). The data is normalized to the spike-in cel-miR-39-3p. Box plots are shown with dots indicating each sample individually to show the variability corresponding to each experimental group. The statistical significance used was 5% and the data were analyzed using the T-test, Wilcoxon test or the median test, as appropriate.

FIGS. 6A, 6B and 6C. ROC curves corresponding to the miRNAs measured in the validation cohort. Using the expression data of the 11 candidate miRNAs measured by RT-qPCR from plasma of patients with breast cancer (n=100) and HD (n=73), the corresponding ROC curves were made using logistic models. Area under the curve (AUC) values, their corresponding confidence intervals (CI) and associated p-values were calculated, which are shown in the table in the lower right corner. The statistical significance used was 5%.

FIGS. 7A, 7B and 7C. Validation of candidate miRNAs in the in-silico external validation cohort. Expression of the indicated circulating miRNAs obtained from public repositories. They were measured by expression microarrays from serum from patients with breast cancer (n=1272) or HD (n=1272). Violin plots were made to show the variability corresponding to each experimental group together with the values of their median and interquartile ranges. The statistical significance used was 5% and the data were analyzed using the T-test, Wilcoxon test or the median test, as appropriate.

FIGS. 8A, 8B and 8C. ROC curves corresponding to the miRNAs measured in the in-silico external validation cohort. Using the expression data of the 11 candidate miRNAs obtained from public repositories, measured by expression microarrays from serum of patients with breast cancer (n=1,272) and HD (n=1,272), the corresponding ROC curves were made using logistic models. Area under the curve (AUC) values, their corresponding confidence intervals (CI) and associated p-values were calculated, which are shown in the table in the lower right corner. The statistical significance used was 5%.

FIG. 9. Graph of the penalty of the coefficients using Lasso Regression. Using the ln of the expression of the candidate miRNAs, the automatic selection of variables was performed using Lasso Regression. On the upper X axis, the number of variables that survive the selection is reported as the value of the Lambda penalty coefficient increases. Each colored line represents a certain variable (miRNA).

FIG. 10. Graph of the optimal number of variables selected automatically by Lasso Regression. Using the in of the expression of the candidate miRNAs, the automatic selection of variables was performed using Lasso Regression. The optimal number of miRNAs at each value of the Lambda penalty coefficient is reported on the upper X-axis (lower X-axis). The blue and orange lines delimit the optimal number of miRNAs automatically selected by mathematical algorithms.

FIG. 11. Ranking graph of miRNAs obtained by Random Forest. Using the ln of the expression of the candidate miRNAs, the automatic selection of variables was performed using Random Forest. The most important miRNAs selected by this technique are reported in descending order. The nodes to make decisions about which subgroup of miRNAs to choose are observed in the distances between the empty circles corresponding to each miRNA in particular.

FIG. 12. Scheme for the selection of predictive models and techniques used. Three techniques were used for the selection of predictive models: Lasso Regression, Random Forest, and selecting the miRNAs that were significant from the model of the 11 candidate miRNAs together. All models were built using machine learning techniques and the best model was selected.

### DETAILED DESCRIPTION

The present disclosure provides methods for the diagnosis and treatment of breast cancer based on the detection of expression levels of specific miRNA biomarker in blood samples from a subject. The methods disclosed herein comprise the determination of expression levels of miRNAs that are overexpressed in subjects with breast cancer and the calculation of scores and/or models based on machine learning techniques. In some aspects, the biomarkers used in the methods of the present disclosure are selected from a panel of miRNAs comprising or consisting of miR-150-5p, miR-106b-3p, miR-106a-5p, miR-125a-5p, miR-17-5p, miR-574-3p, miR-339-5p, miR-339-3p, miR-335-5p, miR-16-5p and miR-21-5p.

Specific subsets of miRNAs from the miRNA panel of the present disclosure have been shown to be particularly sensitive to detect the presence of breast cancer from measurements in circulating blood. In some aspects, the miRNA subset comprises or consists of miR-16-5p, miR-17-5p, miR-106a-5p, and miR-339-3p. In some aspects, the miRNA subset comprises or consists of miR-106a-5p, miR-17-5p, miR-339-3p, miR-16-5p and miR-335-5p. In some aspects, the miRNA subset comprises or consists of miR-106a-5p, miR-17-5p, miR-339-3p, miR-16-5p, miR-150-5p, miR-125a-5p, miR-339-5p, and miR-335-5p. In some aspects, the miRNA subset comprises or consists of miR-150-5p, miR-106b-3p, miR-106a-5p, miR-125a-5p, miR-17-5p, miR-574-3p, miR-339-5p, miR-339-3p, miR-335-5p, miR-16-5p and miR-21-5p. In some aspects, the miRNA subset comprises or consists of miR-150-5p, miR-106b-3p, miR-106a-5p, miR-125a-5p, miR-17-5p, miR-574-3p, miR-339-5p, miR-335-5p, miR-16-5p and miR-21-5p. In some aspects, the miRNA subset comprises or consists of miR-106a-5p, miR-125a-5p, miR-150-5p, miR-17-5p, miR-339-5p, miR-335-5p and miR-16-5p.

The present disclosure provides also predictive models or classifiers to identify patients suitable for treatment with anti-breast cancer therapies, methods to determine whether to initiate, suspend, or modify a treatment, or methods to monitor the prognosis of a patient undergoing anti-cancer therapy. The machine learning models disclosed herein can classify an individual patient into a specific phenotype class. This classification via a score or a model allows patients and cancers to be stratified and guides treatment decision. Thus, the present disclosure provides methods for treating a subject afflicted with breast cancer identified according to the miRNA-based classifiers disclosed herein with a particular therapy. Also provided are personalized treatments that can be administered to a subject having breast cancer. The present disclosure also provides compositions comprising a sample from a subject enriched in the miRNA biomarkers disclosed herein. Also disclosed are kits, detection tests, and systems for the detection of the biomarkers disclosed herein.

The application of the methods and compositions disclosed herein can improve clinical outcomes through early detection of breast cancer and/or by matching patients to therapies.

### I. Definitions

In order that the present disclosure can be more readily understood, certain terms are first defined. As used in this application, except as otherwise expressly provided herein, each of the following terms shall have the meaning set forth below. Additional definitions are set forth throughout the application.

The singular forms “a”, “an” and “the” include plural referents unless the context clearly dictates otherwise. The terms “a” (or “an”), as well as the terms “one or more,” and “at least one” can be used interchangeably herein. In certain aspects, the term “a” or “an” means “single.” In other aspects, the term “a” or “an” includes “two or more” or “multiple.”

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

The terms “about” or “comprising essentially of” refer to a value or composition that is within an acceptable error range for the particular value or composition as determined by one of ordinary skill in the art, which will depend in part on how the value or composition is measured or determined, i.e., the limitations of the measurement system. For example, “about” or “comprising essentially of” can mean within 1 or more than 1 standard deviation per the practice in the art. Alternatively, “about” or “comprising essentially of” can mean a range of up to 10%. Furthermore, particularly with respect to biological systems or processes, the terms can mean up to an order of magnitude or up to 5-fold of a value. When particular values or compositions are provided in the application and claims, unless otherwise stated, the meaning of “about” or “comprising essentially of” should be assumed to be within an acceptable error range for that particular value or composition.

It is understood that wherever aspects are described herein with the language “comprising,” otherwise analogous aspects described in terms of “consisting of” and/or “consisting essentially of” are also provided.

As used herein, the term “approximately,” as applied to one or more values of interest, refers to a value that is similar to a stated reference value. In certain aspects, the term “approximately” refers to a range of values that fall within 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

As described herein, any concentration range, percentage range, ratio range or integer range is to be understood to include the value of any integer within the recited range and, when appropriate, fractions thereof (such as one tenth and one hundredth of an integer), unless otherwise indicated.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their Système International de Unites (SI) accepted form. The headings provided herein are not limitations of the various aspects of the disclosure, which can be had by reference to the specification as a whole. Accordingly, the terms defined are more fully defined by reference to the specification in its entirety.

Abbreviations used herein are defined throughout the present disclosure. Various aspects of the disclosure are described in further detail in the following subsections.

The term “diagnosing” as used herein refers to assessing the probability according to which a subject is afflicted or will be afflicted with a disease or condition referred to in this specification. As will be understood by those skilled in the art, such an assessment is usually not intended to be correct for 100% of the subjects to be diagnosed. The term, however, requires that a statistically significant portion of subjects can be correctly diagnosed to be afflicted with the disease or condition. Whether a portion is statistically significant can be determined without further ado by the person skilled in the art using various well known statistic evaluation tools, e.g., determination of confidence intervals, and p-value determination, e.g. via binomial tests. Details are found in Dowdy and Wearden, Statistics for Research, John Wiley & Sons, New York 1983. Exemplary confidence intervals are at least 90%, at least 95%, at least 97%, at least 98% or at least 99%. The significance levels of statistical tests are, for example, 0.1, 0.05, 0.01, 0.005, or 0.0001. For example, the probability envisaged by the present invention allows that the diagnosis will be correct for at least 60%, at least 70%, at least 80%, or at least 90% of the subjects of a given cohort or population. In some aspects, the diagnostic method has a sufficiently large sensitivity and specificity as described below. In some aspects, the sensitivity envisaged by the present invention allows that the diagnosis of cases will be correct for at least 75%, at least 80%, at least 85%, at least 90%, or at least 95% of the afflicted subjects of a given cohort or population. Also, in some aspects, the specificity envisaged by the present invention allows that the diagnosis will be correct for at least 25%, at least 50%, at least 75%, at least 80%, at least 85%, at least 90%, or at least 95% of the unafflicted subjects of a given cohort or population.

“Administering” refers to the physical introduction of a composition comprising a therapeutic agent (e.g., a monoclonal antibody) to a subject, using any of the various methods and delivery systems known to those skilled in the art. Exemplary routes of administration include intravenous, intramuscular, subcutaneous, intraperitoneal, spinal or other parenteral routes of administration, for example by injection or infusion.

The phrase “parenteral administration” as used herein means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, intraocular, intravitreal, periorbital, epidural and intrasternal injection and infusion, as well as in vivo electroporation. Other non-parenteral routes include an oral, topical, epidermal or mucosal route of administration, for example, intranasally, vaginally, rectally, sublingually or topically. Administering can also be performed, for example, once, a plurality of times, and/or over one or more extended periods.

The terms “treat,” “treating,” and “treatment,” as used herein, refer to any type of intervention or process performed on, or administering an active agent to, the subject with the objective of reversing, alleviating, ameliorating, inhibiting, or slowing down or preventing the progression, development, severity or recurrence of a symptom, complication, condition or biochemical indicia associated with a disease or enhancing overall survival. Treatment can be of a subject having a disease or a subject who does not have a disease (e.g., for prophylaxis). As used here, the terms “treat,” “treating,” and “treatment” refer to the administration of an effective dose or effective dosage.

The term “effective dose” or “effective dosage” is defined as an amount sufficient to achieve or at least partially achieve a desired effect.

A “therapeutically effective amount” or “therapeutically effective dosage” of a drug or therapeutic agent is any amount of the drug that, when used alone or in combination with another therapeutic agent, protects a subject against the onset of a disease or promotes disease regression evidenced by a decrease in severity of disease symptoms, an increase in frequency and duration of disease symptom-free periods, or a prevention of impairment or disability due to the disease affliction.

A therapeutically effective amount or dosage of a drug includes a “prophylactically effective amount” or a “prophylactically effective dosage”, which is any amount of the drug that, when administered alone or in combination with another therapeutic agent to a subject at risk of developing a disease or of suffering a recurrence of disease, inhibits the development or recurrence of the disease.

In addition, the terms “effective” and “effectiveness” with regard to a treatment disclosed herein includes both pharmacological effectiveness and physiological safety. Pharmacological effectiveness refers to the ability of the drug to promote cancer regression in the patient. Physiological safety refers to the level of toxicity, or other adverse physiological effects at the cellular, organ and/or organism level (adverse effects) resulting from administration of the drug.

The ability of a therapeutic agent to promote disease regression, e.g., cancer regression can be evaluated using a variety of methods known to the skilled practitioner, such as in human subjects during clinical trials, in animal model systems predictive of efficacy in humans, or by assaying the activity of the agent in in vitro assays.

By way of example, an “anti-cancer agent” or combination thereof promotes cancer regression in a subject. In some aspects, a therapeutically effective amount of the therapeutic agent promotes cancer regression to the point of eliminating the cancer.

The term “breast cancer” (BC) as used herein relates to an abnormal hyperproliferation of breast tissue cells in a subject. In some aspects, the breast cancer is a primary breast cancer, for example, with a tumor size classification in situ (IS) or pT3, or for example with a tumor size classification of pT1 or pT2.

The term “subject” as referred to herein encompasses animals, for example, mammals such as humans. In some aspects, the subject was in the past afflicted with, is at present afflicted with, is suspected to be afflicted with, or is at risk to be afflicted with breast cancer. Subjects that are afflicted with the said disease can be identified by the accompanying symptoms known for the disease. These symptoms are known in the art and described, e.g., in Breast Cancer Facts & Figures 2011-2012, issued by the American Cancer Society, Inc., Atlanta. However, a subject suspected to be afflicted with the aforementioned disease may also be an apparently healthy subject, e.g., investigated by routine clinical screening, or may be a subject being at risk for developing the aforementioned disease. Risk groups (e.g. individuals with a genetic predisposition to develop breast cancer) for the disease are known in the art and described in, e.g., Dumitrescu & Cotarla, J. Cell. Mol. Med. (2005) 9(1):208-221; Bradbury & Olopade. Rev. Endocr. Metabol. Dis. (2007) 8(3):255-267. In some aspects, the subject is female. In some aspects, the subject is a woman at most 80 years old. In some aspects, the subject is a woman less than 80 years of age.

The term “sample”, as used herein, refers to a sample of a body fluid, to a sample of separated cells or to a sample from a tissue or an organ or to a sample of wash/rinse fluid obtained from an outer or inner body surface. Samples can be obtained by well-known techniques and include, for example, scrapes, swabs or biopsies from the digestive tract, liver, pancreas, anal canal, the oral cavity, the upper aerodigestive tract and the epidermis. Such samples can be obtained by use of brushes, (cotton) swabs, spatula, rinse/wash fluids, punch biopsy devices, puncture of cavities with needles or surgical instrumentation. In some aspects, samples are samples of body fluids, e.g., blood, plasma, serum, urine, saliva, lacrimal fluid, and fluids obtainable from the breast glands, e.g. milk. In some aspects, the samples of body fluids are free of cells of the subject. Tissue or organ samples may be obtained from any tissue or organ by, e.g., biopsy or other surgical procedures. Separated cells may be obtained from the body fluids or the tissues or organs by separating techniques such as filtration, centrifugation or cell sorting. In some aspects, cell, tissue or organ samples are obtained from those body fluids, cells, tissues or organs that are known or suspected to contain the miRNAs of the present disclosure. In some aspects, samples are obtained from those body fluids, cells, tissues or organs described herein below to contain the miRNAs of the present disclosure. In some aspects, the sample is a blood sample, for example a plasma sample, or for example a plasma sample processed as described herein below.

The term “miRNA” or “microRNA” is understood by the skilled artisan and relates to a short ribonucleic acid (RNA) molecule found in eukaryotic cells and in body fluids of metazoan organisms. As used herein interchangeably, a “miR gene product,” “microRNA,” “miR,” or “miRNA” refers to the unprocessed (e.g., precursor) or processed (e.g., mature) RNA transcript from a miR gene. As the miR gene products are not translated into protein, the term “miR gene products” does not include proteins. The unprocessed miR gene transcript is also called a “miR precursor” or “miR prec” and typically comprises an RNA transcript of about 70-100 nucleotides in length. The miR precursor can be processed by digestion with an RNAse (for example, Dicer, Argonaut, or RNAse III (e.g., E. coli RNAse III)) into an active 19-25 nucleotide RNA molecule. This active 19-25 nucleotide RNA molecule is also called the “processed” miR gene transcript or “mature” miRNA.

The term “miR-150-5p” as used herein refers to a human miR-150-5p having the sequence set forth in SEQ ID NO:1. In some aspects, miR-150-5p refers to a human miR-150-5p having the sequence set forth in mirbase.org accession number MIMAT0000451 or any of the sequence reads disclosed therein. In some aspects, miR-150-5p refers to a human miR-150-5p having the sequence set forth in rnacentral.org accession number URS000016FD1A_9606.

The term “miR-106b-3p” as used herein refers to a human miR-106n-3p having the sequence set forth in SEQ ID NO:5. In some aspects, the term miR-106b-3p refers to a human miR-106b-3p having the sequence set forth in mirbase.org accession number MIMAT0004672 or any of the sequence reads disclosed therein. In some aspects, the term miR-106b-3p refers to a human miR-106b-3p having the sequence set forth in rnacentral.org accession number URS0000384021_9606.

The term “miR-106a-5p” as used herein refers to a human miR-106a-5p having the sequence set forth in SEQ ID NO:8. In some aspects, the term miR-106a-5p refers to a human miR-106a-5p having the sequence set forth in mirbase.org accession number MIMAT0000076 or any of the sequence reads disclosed therein. In some aspects, the term miR-106a-5p refers to a human miR-106a-5p having the sequence set forth in rnacentral.org accession number URS000039ED8D_9606.

The term “miR-125a-5p” as used herein refers to a human miR-125a-5p having the sequence set forth in SEQ ID NO:4. In some aspects, the term miR-125a-5p refers to a human miR-125a-5p having the sequence set forth in mirbase.org accession number MIMAT0000443 or any of the sequence reads disclosed therein. In some aspects, the term miR-125a-5p refers to a human miR-125a-5p having the sequence set forth in rnacentral.org accession number URS00005A4DCF_9606.

The term “miR-17-5p” as used herein refers to a human miR-17-5p having the sequence set forth in SEQ ID NO:2. In some aspects, the term miR-17-5p refers to a human miR-17-5p having the sequence set forth in mirbase.org accession number MIMAT0000070 or any of the sequence reads disclosed therein. In some aspects, the term miR-17-5p refers to a human miR-17-5p having the sequence set forth in rnacentral.org accession number URS00002075FA_9606.

The term “miR-574-3p” as used herein refers to a human miR-574-3p having the sequence set forth in SEQ ID NO:3. In some aspects, the term miR-574-3p refers to a human miR-574-3p having the sequence set forth in mirbase.org accession number MIMAT0003239 or any of the sequence reads disclosed therein. In some aspects, the term miR-574-3p refers to a human miR-574-3p having the sequence set forth in rnacentral.org accession number URS00001CF056_9606.

The term “miR-339-5p” as used herein refers to a human miR-339-5p having the sequence set forth in SEQ ID NO:9. In some aspects, the term miR-339-5p refers to a human miR-339-5p having the sequence set forth in mirbase.org accession number MIMAT0000764 or any of the sequence reads disclosed therein. In some aspects, the term miR-339-5p refers to a human miR-339-5p having the sequence set forth in rnacentral.org accession number URS000003FD55_9606.

The term “miR-339-3p” as used herein refers to a human miR-339-3p having the sequence set forth in SEQ ID NO:10. In some aspects, the term miR-339-3p refers to a human miR-339-3p having the sequence set forth in mirbase.org accession number MIMAT0004702 or any of the sequence reads disclosed therein. In some aspects, the term miR-339-3p refers to a human miR-339-3p having the sequence set forth in rnacentral.org accession number URS000055B190_9606.

The term “miR-335-5p” as used herein refers to a human miR-335-5p having the sequence set forth in SEQ ID NO:11. In some aspects, the term miR-335-5p refers to a human miR-335-5p having the sequence set forth in mirbase.org accession number MIMAT0000765 or any of the sequence reads disclosed therein. In some aspects, the term miR-335-5p refers to a human miR-335-5p having the sequence set forth in rnacentral.org accession number URS0000237AF9_9606.

The term “miR-16-5p” as used herein refers to a human miR-16-5p having the sequence set forth in SEQ ID NO:6. In some aspects, the term miR-16-5p refers to a human miR-16-5p having the sequence set forth in mirbase.org accession number MIMAT0000069 or any of the sequence reads disclosed therein. In some aspects, the term miR-16-5p refers to a human miR-16-5p having the sequence set forth in rnacentral.org accession number URS00004BCD9C_9606.

The term “miR-21-5p” as used herein refers to a human miR-21-5p having the sequence set forth in SEQ ID NO:7. In some aspects, the term miR-21-5p refers to a human miR-21-5p having the sequence set forth in mirbase.org accession number MIMAT0000076 or any of the sequence reads disclosed therein. In some aspects, the term miR-21-5p refers to a human miR-21-5p having the sequence set forth in rnacentral.org accession number URS000039ED8D_9606.

The term “cel-miR-39-3p” as used herein refers to a Caenorhabditis elegans cel-miR-39-3p reference miRNA having the sequence set forth in SEQ ID NO:12. In some aspects, the term cel-miR-39-3p refers to a Caenorhabditis elegans cel-miR-39-3p reference miRNA having the sequence set forth in mirbase.org accession number MIMAT0000010 or any of the sequence reads disclosed therein. In some aspects, the term cel-miR-39-3p refers to a Caenorhabditis elegans cel-miR-39-3p reference miRNA having the sequence set forth in rnacentral.org accession number URS00005D4EC7_6239.

The contents of the miRBase and RNAcentral database entries for the accession numbers disclosed above, corresponding to Release 22.1 of miRBase and Release 22 of RNAcentral, are hereby incorporated by reference in their entireties.

It is to be understood that the present disclosure also encompasses pri-miRNAs, and the pre-miRNAs of the miRNAs of the present disclosure. Thus, a miRNA-precursor consists of 25 to several thousand nucleotides, for example 40 to 130 nucleotides, for example 50 to 120 nucleotides, or, for example 60 to 110 nucleotides. In some aspects, a miRNA consists of 5 to 100 nucleotides, for example 10 to 50 nucleotides, or 12 to 40 nucleotides, or 18 to 26 nucleotides. In some aspects, the miRNAs of the present disclosure are miRNAs of human origin, i.e. they are encoded in the human genome. Also, in some aspects, the term miRNA relates to the “guide” strand which eventually enters the RNA-induced silencing complex (RISC) as well as to the “passenger” strand complementary thereto.

### II. Use of miRNA Biomarkers Panels to Detect Breast Cancer

The present disclosure provides methods for the classification of a sample from a subject to determine the likelihood that the subject suffers from breast cancer. As used herein, the term “classifier” refers to a method of sample, subject, or patient classification based on the calculation of one or more signatures, scores, or probabilistic models (e.g., machine learning models) based on the expression levels of a panel of miRNA biomarkers. In some aspects, these classifier are generated using miRNA biomarker panels selected from a full panel comprising, consisting, or consisting essentially of miR-150-5p, miR-106b-3p, miR-106a-5p, miR-125a-5p, miR-17-5p, miR-574-3p, miR-339-5p, miR-339-3p, miR-335-5p, miR-16-5p and miR-21-5p, or from biomarker panels comprising, consisting, or consisting essentially of a subset thereof, e.g., miR-16-5p, miR-17-5p, miR-106a-5p, and miR-339-3p; miR-106a-5p, miR-17-5p, miR-339-3p, miR-16-5p and miR-335-5p; miR-106a-5p, miR-17-5p, miR-339-3p, miR-16-5p, miR-150-5p, miR-125a-5p, miR-339-5p, and miR-335-5p; miR-150-5p, miR-106b-3p, miR-106a-5p, miR-125a-5p, miR-17-5p, miR-574-3p, miR-339-5p, miR-339-3p, miR-335-5p, miR-16-5p and miR-21-5p; miR-150-5p, miR-106b-3p, miR-106a-5p, miR-125a-5p, miR-17-5p, miR-574-3p, miR-339-5p, miR-335-5p, miR-16-5p and miR-21-5p; or, miR-106a-5p, miR-150-5p, miR-125a-5p, miR-17-5p, miR-339-5p, miR-335-5p and miR-16-5p. In some aspects, the classifiers of the present disclosure are predictive models generated by machine learning, e.g., random forests or artificial neural networks. In some aspects, the machine learning classifier is generated using a training set comprising expression data, e.g., microRNA expression data. In some aspects, the classifier, e.g., a machine learning classifier, is generated using fresh samples from subjects. In other aspects, the classifier, e.g., a machine learning classifier, is generated using archival samples.

As used herein, the terms “fresh sample,” “non-archival sample,” and grammatical variants thereof refer to a sample (e.g., a blood sample from a subject having breast cancer, suspected of having breast cancer, or at risk of having breast cancer) which has been processed (e.g., to determine miRNA expression levels) before a predetermined period of time, e.g., one week, after extraction from a subject. In some aspects, a fresh sample has not been frozen. In some aspects, a fresh sample has not been fixed. In some aspects, a fresh sample has been stored for less than two weeks, less than one week, or less than six, five, four, three, or two days before processing. In some specific aspects, the sample is a blood sample that has been maintained for less than 24 hour, 48 hours, or 72 hours at room temperature.

As used herein, the term “archival sample” and grammatical variants thereof refers to a sample (e.g., a blood sample from a subject having breast cancer, suspected of having breast cancer, or at risk of having breast cancer) which has been processed (e.g., to determine miRNA expression levels) after a predetermined period of time, e.g., a week, after extraction from a subject. In some aspects, an archival sample has been frozen. In some aspects, an archival sample has been fixed. In some aspect, an archival sample has a known diagnostic and/or a treatment history. In some aspects, an archival sample has been stored for at least one week, at least one month, at least six months, or at least one year, before processing.

In some aspects, a classifier of the present disclosure comprises, e.g., determining at least one score (e.g., a probability of having breast cancer) determined by measuring the expression levels of a miRNA biomarker panel selected from a full panel comprising, consisting, or consisting essentially of miR-150-5p, miR-106b-3p, miR-106a-5p, miR-125a-5p, miR-17-5p, miR-574-3p, miR-339-5p, miR-339-3p, miR-335-5p, miR-16-5p and miR-21-5p, or from a biomarker panel comprising, consisting, or consisting essentially of a subset thereof, e.g., miR-16-5p, miR-17-5p, miR-106a-5p, and miR-339-3p; miR-106a-5p, miR-17-5p, miR-339-3p, miR-16-5p and miR-335-5p; miR-106a-5p, miR-17-5p, miR-339-3p, miR-16-5p, miR-150-5p, miR-125a-5p, miR-339-5p, and miR-335-5p; miR-150-5p, miR-106b-3p, miR-106a-5p, miR-125a-5p, miR-17-5p, miR-574-3p, miR-339-5p, miR-339-3p, miR-335-5p, miR-16-5p and miR-21-5p; miR-150-5p, miR-106b-3p, miR-106a-5p, miR-125a-5p, miR-17-5p, miR-574-3p, miR-339-5p, miR-335-5p, miR-16-5p and miR-21-5p; or, miR-106a-5p, miR-150-5p, miR-125a-5p, miR-17-5p, miR-339-5p, miR-335-5p and miR-16-5p in a sample obtained from the subject, wherein the at least one score allows assignment of the sample to a particular breast cancer class (e.g., a particular stage in breast cancer). In some aspects, the score is a probability, which can be compared, for example, to a predetermined threshold level.

In some aspects, a classifier of the present disclosure comprises measuring the expression levels of a miRNA biomarker panel selected from a full panel comprising, consisting, or consisting essentially of miR-150-5p, miR-106b-3p, miR-106a-5p, miR-125a-5p, miR-17-5p, miR-574-3p, miR-339-5p, miR-339-3p, miR-335-5p, miR-16-5p and miR-21-5p, or from a biomarker panel comprising, consisting, or consisting essentially of a subset thereof, e.g., miR-16-5p, miR-17-5p, miR-106a-5p, and miR-339-3p; miR-106a-5p, miR-17-5p, miR-339-3p, miR-16-5p and miR-335-5p; miR-106a-5p, miR-17-5p, miR-339-3p, miR-16-5p, miR-150-5p, miR-125a-5p, miR-339-5p, and miR-335-5p; miR-150-5p, miR-106b-3p, miR-106a-5p, miR-125a-5p, miR-17-5p, miR-574-3p, miR-339-5p, miR-339-3p, miR-335-5p, miR-16-5p and miR-21-5p; miR-150-5p, miR-106b-3p, miR-106a-5p, miR-125a-5p, miR-17-5p, miR-574-3p, miR-339-5p, miR-335-5p, miR-16-5p and miR-21-5p; or miR-106a-5p, miR-150-5p, miR-125a-5p, miR-17-5p, miR-339-5p, miR-335-5p and miR-16-5p, in a sample obtained from the subject, and applying a predictive model generated via machine-learning (e.g., a logistic regression, a random forest, an artificial neural network, or a support vector machine model), which assigns the sample to a particular breast cancer class (e.g., a particular stage in breast cancer). In some aspects, the machine-learning model output (e.g., the output from a random forest model) is post-processed using a statistical function that assigns the machine-learning model output to a particular breast cancer class or a combination thereof.

As used herein, the term “breast cancer class” can refer for example to a binary determination, e.g., whether breast cancer in absent or present, or whether the breast cancer is metastatic or non-metastatic, or to a specific factor in breast cancer development, e.g., extent or size of the tumor, spread to nearby lymph nodes, metastasis to distant sites, estrogen receptor status, progesterone receptor status, Her2 status, grade of cancer, or any combination thereof. Once all of these factors have been determined, this information is combined in a process called stage grouping to assign an overall stage. In some aspects, the term breast cancer class refers to a specific stage based on the TNM staging system, wherein T refers to extent or size of the tumor, N refers to spread to nearby lymph nodes, and M refers to metastasis to distant sites.

T followed by a number from 0 to 4 describes the main (primary) tumor's size and if it has spread to the skin or to the chest wall under the breast. Higher T numbers mean a larger tumor and/or wider spread to tissues near the breast. TX: Primary tumor cannot be assessed. T0: No evidence of primary tumor. Tis: Carcinoma in situ (DCIS, or Paget disease of the breast with no associated tumor mass). T1 (includes T1a, T1b, and T1c): Tumor is 2 cm (¾ of an inch) or less across. T2: Tumor is more than 2 cm but not more than 5 cm (2 inches) across. T3: Tumor is more than 5 cm across. T4 (includes T4a, T4b, T4c, and T4d): Tumor of any size growing into the chest wall or skin. This includes inflammatory breast cancer.

N followed by a number from 0 to 3 indicates whether the cancer has spread to lymph nodes near the breast and, if so, how many lymph nodes are involved. Lymph node staging for breast cancer is based on how the nodes look under the microscope, and has changed as technology has improved. Newer methods have made it possible to find smaller and smaller groups of cancer cells, but experts have not been sure how much these tiny deposits of cancer cells influence outlook. A deposit of cancer cells must contain at least 200 cells or be at least 0.2 mm across (less than 1/100 of an inch) for it to change the N stage. An area of cancer spread that is smaller than 0.2 mm (or fewer than 200 cells) does not change the stage, but is recorded with abbreviations (i+ or mol+) that indicate the type of special test used to find the spread. If the area of cancer spread is at least 0.2 mm (or 200 cells), but still not larger than 2 mm, it is called a micrometastasis. Micrometastases are counted only if there are not any larger areas of cancer spread. Areas of cancer spread larger than 2 mm are known to influence outlook and do change the N stage. These larger areas are sometimes called macrometastases, or just called metastases. NX: Nearby lymph nodes cannot be assessed (for example, if they were removed previously). N0: Cancer has not spread to nearby lymph nodes. N0(i+): The area of cancer spread contains fewer than 200 cells and is smaller than 0.2 mm. The abbreviation “i+” means that a small number of cancer cells (isolated tumor cells) were seen in routine stains or when immunohistochemistry was used. N0(mol+): Cancer cells cannot be seen in underarm lymph nodes (even using special stains), but traces of cancer cells were detected using RT-PCR. N1: Cancer has spread to 1 to 3 axillary lymph node(s), and/or cancer is found in internal mammary lymph nodes on sentinel lymph node biopsy. N1mi: Micrometastases in the lymph nodes under the arm. The areas of cancer spread in the lymph nodes are at least 0.2 mm across, but not larger than 2 mm. N1a: Cancer has spread to 1 to 3 lymph nodes under the arm with at least one area of cancer spread greater than 2 mm across. N1b: Cancer has spread to internal mammary lymph nodes on the same side as the cancer, but this spread could only be found on sentinel lymph node biopsy (it did not cause the lymph nodes to become enlarged). N1c: Both N1a and N1b apply. N2: Cancer has spread to 4 to 9 lymph nodes under the arm, or cancer has enlarged the internal mammary lymph nodes N2a: Cancer has spread to 4 to 9 lymph nodes under the arm, with at least one area of cancer spread larger than 2 mm. N2b: Cancer has spread to one or more internal mammary lymph nodes, causing them to become enlarged. N3: Any of the following N3x classes: N3a: either: cancer has spread to 10 or more axillary lymph nodes, with at least one area of cancer spread greater than 2 mm, or cancer has spread to the lymph nodes under the collarbone (infraclavicular nodes), with at least one area of cancer spread greater than 2 mm. N3b: either: cancer is found in at least one axillary lymph node (with at least one area of cancer spread greater than 2 mm) and has enlarged the internal mammary lymph nodes, or cancer has spread to 4 or more axillary lymph nodes (with at least one area of cancer spread greater than 2 mm), and to the internal mammary lymph nodes on sentinel lymph node biopsy. N3c: Cancer has spread to the lymph nodes above the collarbone (supraclavicular nodes) on the same side of the cancer with at least one area of cancer spread greater than 2 mm.

M followed by a 0 or 1 indicates whether the cancer has spread to distant organs—for example, the lungs, liver, or bones. M0: No distant spread is found on x-rays (or other imaging tests) or by physical exam. cM0(i+): Small numbers of cancer cells are found in blood or bone marrow (found only by special tests), or tiny areas of cancer spread (no larger than 0.2 mm) are found in lymph nodes away from the underarm, collarbone, or internal mammary areas. M1: Cancer has spread to distant organs (most often to the bones, lungs, brain, or liver) as seen on imaging tests or by physical exam, and/or a biopsy of one of these areas proves cancer has spread and is larger than 0.2 mm.

In some aspects, the classifiers disclosed herein can assign a sample obtained from a subject to a specific T, N, or M stage, or any combination thereof.

After a classifier of the present disclosure, e.g., a classifier based on the assignment of a score, e.g., a probability score, or a machine-learning classifier based, e.g., on a probabilistic model, assigns the subject's sample to a particular breast cancer class, such classification would guide the selection and administration of a specific treatment or treatments which have been determined to be effective to treat the same type of cancer in other subjects having the same breast cancer class, e.g., a breast cancer therapy disclosed below or a combination thereof.

The term “score” as used herein refers to a numerical value or other representation which is linked or based on a specific feature, e.g. a Z score that integrates expression values obtained from a number of genes or miRNA, after assigning specific weights to each value. In some aspects, a numeric scored can be compared to a “cutoff value” or “threshold,” which as used herein means a numerical value or other representation whose value is used to arbitrate between two or more states (e.g. diseased and non-diseased) of classification for a biological sample. For example, if a parameter is greater than the cutoff value, a first classification of the quantitative data is made (e.g. diseased state); or if the parameter is less than the cutoff value, a different classification of the quantitative data is made (e.g. non-diseased state).

The classifiers disclosed herein can be used to assign a patient or a cancer sample to a specific treatment class. Specific subpopulations of patients can be further classified according to the classifiers disclosed herein by using, for example, more than one threshold. In some aspects, splitting the output of a probability score or machine-learning model, combined for example with the use of different subpanels of the miRNAs disclosed herein can yield a combined biomarker comprising a single final score or a combination thereof. E.g., specific thresholds in the probability output may provide a likelihood of biomarker positivity or biomarker negativity corresponding to T, N and M stages.

In other aspects, probability scores and/or machine-learning models generated using the miRNA panels disclosed herein may provide distinct T, N, and M classifications, which can be combined into a single combined biomarker. For example, a first probability score or machine-learning model derived from a miRNA subpanel A may yield a first biomarker corresponding to T staging, a second probability score or machine-learning model derived from a miRNA subpanel B may yield a second biomarker corresponding to N staging, a third probability score or machine-learning model derived from a miRNA subpanel C may yield a third biomarker corresponding to M staging, and finally the first, second, and third biomarker may the integrated into a combined biomarker, i.e., a biomarker derived from discrete biomarkers.

In some aspects, the output of the classifiers disclosed herein can be combined with other biomarkers known in the art, e.g., BRCA status, or with biomarkers related to the subject physiology (e.g., pre-existing conditions) or lifestyle. In turn, the classifiers disclosed herein, alone or in combination with other classifiers, will inform a clinician (e.g., a medical doctor), e.g., to decide whether a patient should be selected for treatment, whether a treatment should be initiated, whether treatment should be suspended, or whether treatment should be modified.

The classifiers disclosed herein rely on the selection of a specific miRNA biomarker panel as the source of the input data used by the classifier. In some aspects, each one of the miRNAs in a miRNA biomarker panel is referred to as a biomarker. The “level” of a miRNA biomarker disclosed herein or a combination thereof can refer, in some instances, to the “expression level” of the biomarker, e.g., the level of miRNA biomarker in a sample. In some aspects, the expression level of an mRNA biomarker disclosed herein can be quantified using PCR (e.g., real-time PCR), sequencing (e.g., deep sequencing or next generation sequencing, e.g., RNA-Seq), or microarray expression profiling or other technologies that utilize RNAse protection in combination with amplification or amplification and new quantitation methods such as RNA-Seq or other methods. In a specific aspect, the expression levels of the miRNAs disclosed herein are detected using techniques selected from the group consisting of quantitative real-time PCR (qPCR), miRNA expression microarrays, and DNA biosensors. In specific aspects of the present disclosure, the expression levels of the miRNA biomarkers disclosed herein (e.g., miR-150-5p, miR-106b-3p, miR-106a-5p, miR-125a-5p, miR-17-5p, miR-574-3p, miR-339-5p, miR-339-3p, miR-335-5p, miR-16-5p and miR-21-5p) are measured in a blood simple from a subject, e.g., a subject suspected of having breast cancer, a subject having breast cancer, or a subject at risk of having breast cancer.

In the classifiers disclosed herein, expression levels for miRNAs in a miRNA biomarker panel of the present disclosure can be used to classify a sample as, e.g., breast cancer positive or breast cancer negative, according to whether a calculated score (e.g., a probability score) is above or below a certain threshold value.

In the classifiers disclosed herein, expression levels for miRNAs in a miRNA biomarker panel of the present disclosure and their assignment, e.g., to presence or absence of breast cancer can be used as a training set for machine-learning, e.g., using random forests or an artificial neural network (ANN). The machine learning would yield a model, e.g., a random forest model. Subsequently, expression levels for miRNAs in a miRNA biomarker panel obtained from a sample or samples from a test subject would be used as input for the model, which would classify the subject's sample as, e.g., breast cancer positive or breast cancer negative.

Biomarker panels: The present disclosure provides miRNA biomarker panels for the detection of breast cancer. In some aspects, the miRNA biomarker panel comprises, consists, or consists essentially of miR-150-5p, miR-106b-3p, miR-106a-5p, miR-125a-5p, miR-17-5p, miR-574-3p, miR-339-5p, miR-339-3p, miR-335-5p, miR-16-5p and miR-21-5p. In some aspects, the miRNA biomarker panel comprises at least one, at least two, at least three, at least four, at least five, at least six, at least seven, at least eight, at least nine, or least 10 miRNA selected from the group consisting of miR-150-5p, miR-106b-3p, miR-106a-5p, miR-125a-5p, miR-17-5p, miR-574-3p, miR-339-5p, miR-339-3p, miR-335-5p, miR-16-5p and miR-21-5p.

In some aspects, a miRNA biomarker panel of the present disclosure comprises two miRNAs selected from the group consisting of miR-150-5p, miR-106b-3p, miR-106a-5p, miR-125a-5p, miR-17-5p, miR-574-3p, miR-339-5p, miR-339-3p, miR-335-5p, miR-16-5p and miR-21-5p. In some aspects, a miRNA biomarker panel of the present disclosure consists of two miRNAs selected from the group consisting of miR-150-5p, miR-106b-3p, miR-106a-5p, miR-125a-5p, miR-17-5p, miR-574-3p, miR-339-5p, miR-339-3p, miR-335-5p, miR-16-5p and miR-21-5p.

In some aspects, a miRNA biomarker panel of the present disclosure comprises three miRNAs selected from the group consisting of miR-150-5p, miR-106b-3p, miR-106a-5p, miR-125a-5p, miR-17-5p, miR-574-3p, miR-339-5p, miR-339-3p, miR-335-5p, miR-16-5p and miR-21-5p. In some aspects, a miRNA biomarker panel of the present disclosure consists of three miRNAs selected from the group consisting of miR-150-5p, miR-106b-3p, miR-106a-5p, miR-125a-5p, miR-17-5p, miR-574-3p, miR-339-5p, miR-339-3p, miR-335-5p, miR-16-5p and miR-21-5p.

In some aspects, a miRNA biomarker panel of the present disclosure comprises four miRNAs selected from the group consisting of miR-150-5p, miR-106b-3p, miR-106a-5p, miR-125a-5p, miR-17-5p, miR-574-3p, miR-339-5p, miR-339-3p, miR-335-5p, miR-16-5p and miR-21-5p. In some aspects, a miRNA biomarker panel of the present disclosure consists of four miRNAs selected from the group consisting of miR-150-5p, miR-106b-3p, miR-106a-5p, miR-125a-5p, miR-17-5p, miR-574-3p, miR-339-5p, miR-339-3p, miR-335-5p, miR-16-5p and miR-21-5p.

In some aspects, a miRNA biomarker panel of the present disclosure comprises five miRNAs selected from the group consisting of miR-150-5p, miR-106b-3p, miR-106a-5p, miR-125a-5p, miR-17-5p, miR-574-3p, miR-339-5p, miR-339-3p, miR-335-5p, miR-16-5p and miR-21-5p. In some aspects, a miRNA biomarker panel of the present disclosure consists of five miRNAs selected from the group consisting of miR-150-5p, miR-106b-3p, miR-106a-5p, miR-125a-5p, miR-17-5p, miR-574-3p, miR-339-5p, miR-339-3p, miR-335-5p, miR-16-5p and miR-21-5p.

In some aspects, a miRNA biomarker panel of the present disclosure comprises six miRNAs selected from the group consisting of miR-150-5p, miR-106b-3p, miR-106a-5p, miR-125a-5p, miR-17-5p, miR-574-3p, miR-339-5p, miR-339-3p, miR-335-5p, miR-16-5p and miR-21-5p. In some aspects, a miRNA biomarker panel of the present disclosure consists of six miRNAs selected from the group consisting of miR-150-5p, miR-106b-3p, miR-106a-5p, miR-125a-5p, miR-17-5p, miR-574-3p, miR-339-5p, miR-339-3p, miR-335-5p, miR-16-5p and miR-21-5p.

In some aspects, a miRNA biomarker panel of the present disclosure comprises seven miRNAs selected from the group consisting of miR-150-5p, miR-106b-3p, miR-106a-5p, miR-125a-5p, miR-17-5p, miR-574-3p, miR-339-5p, miR-339-3p, miR-335-5p, miR-16-5p and miR-21-5p. In some aspects, a miRNA biomarker panel of the present disclosure consists of seven miRNAs selected from the group consisting of miR-150-5p, miR-106b-3p, miR-106a-5p, miR-125a-5p, miR-17-5p, miR-574-3p, miR-339-5p, miR-339-3p, miR-335-5p, miR-16-5p and miR-21-5p.

In some aspects, a miRNA biomarker panel of the present disclosure comprises eight miRNAs selected from the group consisting of miR-150-5p, miR-106b-3p, miR-106a-5p, miR-125a-5p, miR-17-5p, miR-574-3p, miR-339-5p, miR-339-3p, miR-335-5p, miR-16-5p and miR-21-5p. In some aspects, a miRNA biomarker panel of the present disclosure consists of eight miRNAs selected from the group consisting of miR-150-5p, miR-106b-3p, miR-106a-5p, miR-125a-5p, miR-17-5p, miR-574-3p, miR-339-5p, miR-339-3p, miR-335-5p, miR-16-5p and miR-21-5p.

In some aspects, a miRNA biomarker panel of the present disclosure comprises nine miRNAs selected from the group consisting of miR-150-5p, miR-106b-3p, miR-106a-5p, miR-125a-5p, miR-17-5p, miR-574-3p, miR-339-5p, miR-339-3p, miR-335-5p, miR-16-5p and miR-21-5p. In some aspects, a miRNA biomarker panel of the present disclosure consists of nine miRNAs selected from the group consisting of miR-150-5p, miR-106b-3p, miR-106a-5p, miR-125a-5p, miR-17-5p, miR-574-3p, miR-339-5p, miR-339-3p, miR-335-5p, miR-16-5p and miR-21-5p.

In some aspects, a miRNA biomarker panel of the present disclosure comprises ten miRNAs selected from the group consisting of miR-150-5p, miR-106b-3p, miR-106a-5p, miR-125a-5p, miR-17-5p, miR-574-3p, miR-339-5p, miR-339-3p, miR-335-5p, miR-16-5p and miR-21-5p. In some aspects, a miRNA biomarker panel of the present disclosure consists of ten miRNAs selected from the group consisting of miR-150-5p, miR-106b-3p, miR-106a-5p, miR-125a-5p, miR-17-5p, miR-574-3p, miR-339-5p, miR-339-3p, miR-335-5p, miR-16-5p and miR-21-5p.

In some aspects, a miRNA biomarker panel of the present disclosure comprises miR-106a-5p, miR-17-5p, miR-339-3p, miR-16-5p, miR-150-5p, miR-125a-5p, miR-339-5p, and miR-335-5p. In some aspects, a miRNA biomarker panel of the present disclosure consists of miR-106a-5p, miR-17-5p, miR-339-3p, miR-16-5p, miR-150-5p, miR-125a-5p, miR-339-5p, and miR-335-5p. In some aspects, a miRNA biomarker panel of the present disclosure consists of miR-106a-5p, miR-17-5p, miR-339-3p, miR-16-5p, miR-150-5p, miR-125a-5p, miR-339-5p, and miR-335-5p, and one, two, three, four, five, six, seven, eight, nine, or ten additional miRNAs, e.g., miRNAs disclosed herein.

In some aspects, a miRNA biomarker panel of the present disclosure comprises miR-106a-5p, miR-339-3p, miR-16-5p, miR-150-5p and miR-339-5p. In some aspects, a miRNA biomarker panel of the present disclosure consists of miR-106a-5p, miR-339-3p, miR-16-5p, miR-150-5p and miR-339-5p. In some aspects, a miRNA biomarker panel of the present disclosure consists of miR-106a-5p, miR-339-3p, miR-16-5p, miR-150-5p and miR-339-5p, and one, two, three, four, five, six, seven, eight, nine, or ten additional miRNAs, e.g., miRNAs disclosed herein.

In some aspects, a miRNA biomarker panel of the present disclosure comprises miR-106a-5p, miR-17-5p, miR-339-3p, miR-16-5p and miR-335-5p. In some aspects, a miRNA biomarker panel of the present disclosure consists of miR-106a-5p, miR-17-5p, miR-339-3p, miR-16-5p and miR-335-5p. In some aspects, a miRNA biomarker panel of the present disclosure consists of miR-106a-5p, miR-17-5p, miR-339-3p, miR-16-5p and miR-335-5p, and one, two, three, four, five, six, seven, eight, nine, or ten additional miRNAs, e.g., mRNAs disclosed herein.

In some aspects, a miRNA biomarker panel of the present disclosure comprises miR-106a-5p, miR-17-5p, miR-339-3p, and miR-16-5p. In some aspects, a miRNA biomarker panel of the present disclosure consists of miR-106a-5p, miR-17-5p, miR-339-3p, and miR-16-5p. In some aspects, a miRNA biomarker panel of the present disclosure consists of miR-106a-5p, miR-17-5p, miR-339-3p, and miR-16-5p, and one, two, three, four, five, six, seven, eight, nine, or ten additional miRNAs, e.g., miRNAs disclosed herein.

In some aspects, a miRNA biomarker panel of the present disclosure comprises miR-150-5p, miR-106b-3p, miR-106a-5p, miR-125a-5p, miR-17-5p, miR-574-3p, miR-339-5p, miR-335-5p, miR-16-5p. and miR-21-5p. In some aspects, a miRNA biomarker panel of the present disclosure consists of miR-150-5p, miR-106b-3p, miR-106a-5p, miR-125a-5p, miR-17-5p, miR-574-3p, miR-339-5p, miR-335-5p, miR-16-5p. and miR-21-5p. In some aspects, a miRNA biomarker panel of the present disclosure consists of miR-150-5p, miR-106b-3p, miR-106a-5p, miR-125a-5p, miR-17-5p, miR-574-3p, miR-339-5p, miR-335-5p, miR-16-5p. and miR-21-5p, and one, two, three, four, five, six, seven, eight, nine, or ten additional miRNAs, e.g., miRNAs disclosed herein.

In some aspects, a miRNA biomarker panel of the present disclosure comprises miR-106a-5p, miR-150-5p, miR-125a-5p, miR-17-5p, miR-339-5p, miR-335-5p and miR-16-5p. In some aspects, a miRNA biomarker panel of the present disclosure consists of miR-106a-5p, miR-150-5p, miR-125a-5p, miR-17-5p, miR-339-5p, miR-335-5p and miR-16-5p. In some aspects, a miRNA biomarker panel of the present disclosure consists of miR-106a-5p, miR-150-5p, miR-125a-5p, miR-17-5p, miR-339-5p, miR-335-5p and miR-16-5p, and one, two, three, four, five, six, seven, eight, nine, or ten additional miRNAs, e.g., miRNAs disclosed herein.

In some aspects, the miRNA biomarker panel used in the methods disclosed herein does not consist of miR-125b-2, miR-125b-1, miR-10b, miR-181a, miR-140, miR-21, miR-29a prec, miR-199b, miR-29b-1, miR-130a, miR-155, let7a-2, miR-29c, miR-224, miR-31, miR-122a, miR-16-2, miR-145, miR-205, miR-100, miR-30c, miR-17-5p, miR-29b-2, miR-146, and miR-181b-1, or a combination thereof. In some aspects, the miRNA biomarker panel used in the methods disclosed herein does not comprise miR-125b-2, miR-125b-1, miR-10b, miR-181a, miR-140, miR-21, miR-29a prec, miR-199b, miR-29b-1, miR-130a, miR-155, let7a-2, miR-29c, miR-224, miR-31, miR-122a, miR-16-2, miR-145, miR-205, miR-100, miR-30c, miR-17-5p, miR-29b-2, miR-146, and miR-181b-1, or a combination thereof.

In some aspects, the miRNA biomarker panel used in the methods disclosed herein does not consist of miRNAs miR-146a, miR-155, miR-222 and miR-339, or a combination thereof. In some aspects, the miRNA biomarker panel used in the methods disclosed herein does not comprise miRNAs miR-146a, miR-155, miR-222 and miR-339, or a combination thereof.

In some aspects, the miRNA biomarker panel used in the methods disclosed herein does not consist of miRNA 429-3p, miRNA 29c-3p, miRNA 29a-3p, miRNA 29b-3p, miRNA 200a-3p, miRNA 200b-3p, miRNA 200c-3p, miRNA 141-3p, miRNA 15a-5p, miRNA 15b-5p, miRNA 16-5p, miRNA 424-5p, miRNA 497-5p, miRNA 615-3p, miRNA 451a-3p and miRNA 542-5p, or a combination thereof. In some aspects, the miRNA biomarker panel used in the methods disclosed herein does not comprise miRNA 429-3p, miRNA 29c-3p, miRNA 29a-3p, miRNA 29b-3p, miRNA 200a-3p, miRNA 200b-3p, miRNA 200c-3p, miRNA 141-3p, miRNA 15a-5p, miRNA 15b-5p, miRNA 16-5p, miRNA 424-5p, miRNA 497-5p, miRNA 615-3p, miRNA 451a-3p and miRNA 542-5p, or a combination thereof.

In some aspects, the miRNA biomarker panel used in the methods disclosed herein does not consist of miR-183 and/or miR-494. In some aspects, the miRNA biomarker panel used in the methods disclosed herein does not comprise miR-183 and/or miR-494.

In some aspects, the miRNA biomarker panel used in the methods disclosed herein does not consist of miR-5p, miR-10b-5p, and miR-99a-5p, or a combination thereof. In some aspects, the miRNA biomarker panel used in the methods disclosed herein does not comprise miR-5p, miR-10b-5p, and miR-99a-5p, or a combination thereof

In some aspects, the miRNA biomarker panel used in the methods disclosed herein does not consist of miR-409-3, miR-382-5p, miR-375 and miR-23a-3p, or a combination thereof. In some aspects, the miRNA biomarker panel used in the methods disclosed herein does not comprise miR-409-3, miR-382-5p, miR-375 and miR-23a-3p, or a combination thereof.

In some aspects, the miRNA biomarker panel used in the methods disclosed herein does not consist of let-7b-5p, miR-106a-5p, miR-16-5p, miR-19a-3p, miR-19b-3p, miR-20a-5p, miR-223-3p, miR-25-3p, miR-425-5p, miR-451a, miR-92a-3p and miR-93-5p, or a combination thereof. In some aspects, the miRNA biomarker panel used in the methods disclosed herein does not comprise let-7b-5p, miR-106a-5p, miR-16-5p, miR-19a-3p, miR-19b-3p, miR-20a-5p, miR-223-3p, miR-25-3p, miR-425-5p, miR-451a, miR-92a-3p and miR-93-5p, or a combination thereof.

In some aspects, the miRNA biomarker panel used in the methods disclosed herein does not consist of miR-139-3p, miR-193a-3p, miR-206, miR-519a, miR-526b, miR-571c, miR-571, miR-148b, miR-184, miR-376c, miR-409-3p, miR-424 and miR-801, or a combination thereof. In some aspects, the miRNA biomarker panel used in the methods disclosed herein does not comprise miR-139-3p, miR-193a-3p, miR-206, miR-519a, miR-526b, miR-571c, miR-571, miR-148b, miR-184, miR-376c, miR-409-3p, miR-424 and miR-801, or a combination thereof.

In some aspects, the miRNA biomarker panel used in the methods disclosed herein does not consist of miR-149-5p, miR-10a-5p, miR-20b-5p, miR-30a-3p and miR-342-5p, or a combination thereof. In some aspects, the miRNA biomarker panel used in the methods disclosed herein does not comprise miR-149-5p, miR-10a-5p, miR-20b-5p, miR-30a-3p and miR-342-5p, or a combination thereof.

Sample preparation and processing: The methods disclosed herein comprise measuring the expression levels of a miRNA biomarker panel selected from a sample, e.g., a biological sample obtained from a subject. Biomarker levels (e.g., expression levels of miRNAs in a miRNA biomarker panel of the present disclosure) can be measured in any biological sample that contains or is suspected to contain one or more of the biomarkers disclosed herein, including any tissue sample or biopsy from the subject or a blood sample, e.g., a venous blood sample. In some aspects, the sample is a cell-free sample, e.g., comprising cell-free nucleic acids (e.g., miRNAs). A sample can comprise, in some instances, compounds that are not naturally intermixed with the tissue in nature such as preservatives, anticoagulants, buffers, fixatives, nutrients, antibiotics or the like. In some aspects, the present disclosure provides a sample that has been enriched in the miRNAs of the miRNA biomarker panels of the present disclosure. In some aspects, the level of miRNAs corresponding to a miRNA biomarker panel of the present disclosure is enriched with respect to other miRNAs present in the original sample.

In some aspects, the sample has been enriched in nucleic acids in general. In some aspects, the sample has been deproteinized. In some aspects, the sample has been processed, e.g., by centrifugation to remove cells and/or protein aggregated. In some aspects, the sample has been enriched in miRNAs using an affinity binding method, for example kits including columns, TRizol or any similar reagent that contains guanidinium thiocyanate and phenol, including homemade reagents that allow RNA isolation. Concentration and quantification of mRNAs can be conducted using any methods known in the art. See, e.g., Bissels et al. (2009) RNA 15(12):2375-2384; Wang et al. (2012) PLoS One 7(7):e41561; Bosson et al. (2014) Molecular Cell 56:347-359; Cheung et al. (2018) Biomicrofluidics 12:014104; Ustuner et al. (2021) Scientific Reports 11:19650; which are herein incorporated by reference in their entireties.

In some aspect, the amount of miRNAs in an enriched sample is at least about 100%, at least about 200%, at least about 300%, at least about 400%, at least about 500%, at least about 600%, at least about 700%, at least about 800%, at least about 900%, or at least about 1000% higher than the level of miRNAs in the original sample.

In some aspect, the amount of miRNAs in an enriched sample is about 100%, about 200%, about 300%, about 400%, about 500%, about 600%, about 700%, about 800%, about 900%, or about 1000% higher than the level of miRNAs in the original sample.

In some aspect, the amount of miRNAs in an enriched sample is between about 100% and about 200%, about 200% and 300%, about 300% and about 400%, about 400% and about 500%, about 500% and about 600%, about 600% and about 700%, about 700% and about 800%, about 800% and about 900%, about 900% and about 1000%, about 100% and about 1000%, about 200% and about 500%, about 100% and about 300%, about 400% and about 800%, or about 500% and about 1000% higher than the level of miRNAs in the original sample.

In some aspect, the amount of miRNAs in an enriched sample is at least about 2-fold, at least about 3-fold, at least about 4-fold, at least about 5-fold, at least about 6-fold, at least about 7-fold, at least about 8-fold, at least about 9-fold, or at least about 10-fold higher than the level of miRNAs in the original sample.

In some aspect, the amount of miRNAs in an enriched sample is about 2-fold, about 3-fold, about 4-fold, about 5-fold, about 6-fold, about 7-fold, about 8-fold, about 9-fold, or about 10-fold higher than the level of miRNAs in the original sample.

In some aspect, the amount of miRNAs in an enriched sample is between about 2-fold and about 3-fold, about 3-fold and about 4-fold, about 4-fold and about 5-fold, about 5-fold and about 6-fold, about 6-fold and about 7-fold, about 7-fold and about 8-fold, about 8-fold and about 9-fold, about 9-fold and about 10-fold, about 1-fold and about 10-fold, about 2-fold and about 5-fold, about 1-fold and about 3-fold, about 4-fold and about 8-fold, or about 5-fold and about 10-fold higher than the level of miRNAs in the original sample.

miRNA biomarker expression levels: The level of expression of the genes in the gene panels described herein can be determined using any method in the art. For example, the most commonly used techniques for miRNA quantification are real-time quantitative PCR (qPCR), microarray, and sequencing (miRNA-seq). Other types of techniques to quantify specific miRNAs are miRNA-seq, miRNA expression microarrays, and DNA biosensors. In some specific aspects, the quantification technique is Stem Loop RT-qPCR, but any of the other techniques known in the art could also be used for its quantification. A person skilled in the art could routinely fine-tune each of these quantification techniques to determine the level of expression of the miRNAs of the present disclosure.

In some aspects, the miRNA levels are determined using sequencing methods, e.g., Next Generation Sequencing (NGS). In some aspects, the NGS is RNA-Seq, EdgeSeq, PCR, Nanostring, or any combination thereof, or any technologies that can measure miRNA. In some aspects, the miRNA measurement methods comprise nuclease protection.

In some aspects, the miRNA levels are determined using fluorescence. In some aspects, the miRNA levels are determined using an Affymetrix microarray or a microarray such as sold by Agilent. Any method of sequencing known in the art can be used.

Sequencing of nucleic acids isolated by selection methods are typically carried out using next-generation sequencing (NGS). Next-generation sequencing includes any sequencing method that determines the nucleotide sequence of either individual nucleic acid molecules or clonally expanded proxies for individual nucleic acid molecules in a highly parallel fashion (e.g., greater than 105 molecules are sequenced simultaneously). In one aspect, the relative abundance of the nucleic acid species in the library can be estimated by counting the relative number of occurrences of their cognate sequences in the data generated by the sequencing experiment. Next generation sequencing methods are known in the art, and are described, e.g., in Metzker, M. (2010) Nature Biotechnology Reviews 11:31-46; Eastel et al. (2019) Expert Rev. Mol. Diag. 19:591-98; and, McCombie et al. (2019) Cold Spring Harb. Perspect. Med. 9:a036798; which are herein incorporated by reference in their entireties.

In some aspects, next-generation sequencing allows for the determination of the nucleotide sequence of an individual nucleic acid biomarker (e.g., Helicos BioSciences' HeliScope Gene Sequencing system, and Pacific Biosciences' PacBio RS system). In other aspects, the sequencing method determines the nucleotide sequence of clonally expanded proxies for individual nucleic acid biomarkers and/or quantification of the level (e.g., relative quantity of copies) of individual nucleic acid biomarkers, e.g., miRNA biomarkers of the present disclosure (e.g., the Solexa sequencer, Illumina Inc., San Diego, Calif; 454 Life Sciences (Branford, Conn.), and Ion Torrent), e.g., massively parallel short-read sequencing (e.g., the Solexa sequencer, Illumina Inc., San Diego, Calif.), which generates more bases of sequence per sequencing unit than other sequencing methods that generate fewer but longer reads. Other methods or machines for next-generation sequencing include, but are not limited to, the sequencers provided by 454 Life Sciences (Branford, Conn.), Applied Biosystems (Foster City, Calif.; SOLiD sequencer), Helicos BioSciences Corporation (Cambridge, Mass.), and emulsion and microfluidic sequencing technology nanodroplets (e.g., GnuBio droplets).

Platforms for next-generation sequencing include, but are not limited to, Roche/454's Genome Sequencer (GS) FLX System, Illumina/Solexa's Genome Analyzer (GA), Life/APG's Support Oligonucleotide Ligation Detection (SOLiD) system, Polonator's G.007 system, Helicos BioSciences' HeliScope Gene Sequencing system, and Pacific Biosciences' PacBio RS system, HTG Molecular Diagnostics' EdgeSeq, and Nanostring Technology's Hyb & Seq NGS Technology.

NGS technologies can include one or more steps, e.g., template preparation, sequencing and imaging, and data analysis, which are disclosed more in detail below.

It is noted that template amplification methods, such as PCR methods known in the art, can also be used to quantify biomarker levels. Exemplary template enrichment methods include, e.g., microdroplet PCR technology (Tewhey et al., Nature Biotech. 2009, 27:1025-1031), custom-designed oligonucleotide microarrays (e.g., Roche/NimbleGen oligonucleotide microarrays), and solution-based hybridization methods (e.g., molecular inversion probes (MIPs) (Porreca et al., Nature Methods, 2007, 4:931-936; Krishnakumar et al., Proc. Natl. Acad. Sci. USA, 2008, 105:9296-9310; Turner et al., Nature Methods, 2009, 6:315-316), and biotinylated RNA capture sequences (Gnirke et al., Nat. Biotechnol. 2009; 27(2):182-9).

In some aspects, the expression levels of a plurality of miRNA biomarkers of the present disclosure, e.g., a full panel comprising, consisting, or consisting essentially of miR-150-5p, miR-106b-3p, miR-106a-5p, miR-125a-5p, miR-17-5p, miR-574-3p, miR-339-5p, miR-339-3p, miR-335-5p, miR-16-5p and miR-21-5p, or a biomarker panel comprising, consisting, or consisting essentially of a subset thereof, e.g., miR-16-5p, miR-17-5p, miR-106a-5p, and miR-339-3p; miR-106a-5p, miR-17-5p, miR-339-3p, miR-16-5p and miR-335-5p; miR-106a-5p, miR-17-5p, miR-339-3p, miR-16-5p, miR-150-5p, miR-125a-5p, miR-339-5p, and miR-335-5p; miR-150-5p, miR-106b-3p, miR-106a-5p, miR-125a-5p, miR-17-5p, miR-574-3p, miR-339-5p, miR-339-3p, miR-335-5p, miR-16-5p and miR-21-5p; miR-150-5p, miR-106b-3p, miR-106a-5p, miR-125a-5p, miR-17-5p, miR-574-3p, miR-339-5p, miR-335-5p, miR-16-5p and miR-21-5p; or, miR-106a-5p, miR-150-5p, miR-125a-5p, miR-17-5p, miR-339-5p, miR-335-5p and miR-16-5p, are normalized with respect to a control. In some aspects, the control in an exogenous miRNA, e.g., cel-miR-39 (cel329), cel-miR-54 or cel-miR-238 from Caenorhabditis elegans, or a combination thereof. In other aspects, the control is an endogenous miRNA or a combination thereof, e.g., the averaged Cq value of all the analyzed miRNAs (global mean). In some aspects, the control is a stable endogenous miRNA identified, for example, by using geNorm, NormFinder, or BestKeeper. See, e.g., Faraldi et al. (2019) Scientific Reports 9: 1584.

Classifiers: The classifiers of the present disclosure rely on the integration of the expression levels of a plurality of miRNAs to derive, e.g., a score (e.g., a Z-score) or a probabilistic model (e.g., derived from a machine learning technique such as random forests) which is correlated with the presence or absence of breast cancer, and/or correlations with responses to particular anticancer therapies. Thus, the determination that a subject from a sample (e.g., a blood sample) has a particular score (e.g., a Z-score or a probability scores obtained by applying a machine learning model) allows determining whether the subject has breast cancer, and/or the selection of the appropriate treatment or combination thereof. Thus, in one aspect, the present disclosure provides methods for determining the presence or absence of breast cancer in a subject in need thereof wherein the method comprises determining a combined biomarker which comprises a score (e.g., a Z-score or a probability scores obtained by applying a machine learning model) derived from expression levels of a plurality of miRNA biomarkers of the present disclosure, e.g., a full panel comprising, consisting, or consisting essentially of miR-150-5p, miR-106b-3p, miR-106a-5p, miR-125a-5p, miR-17-5p, miR-574-3p, miR-339-5p, miR-339-3p, miR-335-5p, miR-16-5p and miR-21-5p, or a biomarker panel comprising, consisting, or consisting essentially of a subset thereof, e.g., miR-16-5p, miR-17-5p, miR-106a-5p, and miR-339-3p; miR-106a-5p, miR-17-5p, miR-339-3p, miR-16-5p and miR-335-5p; miR-106a-5p, miR-17-5p, miR-339-3p, miR-16-5p, miR-150-5p, miR-125a-5p, miR-339-5p, and miR-335-5p; miR-150-5p, miR-106b-3p, miR-106a-5p, miR-125a-5p, miR-17-5p, miR-574-3p, miR-339-5p, miR-339-3p, miR-335-5p, miR-16-5p and miR-21-5p; miR-150-5p, miR-106b-3p, miR-106a-5p, miR-125a-5p, miR-17-5p, miR-574-3p, miR-339-5p, miR-335-5p, miR-16-5p and miR-21-5p; or miR-150-5p, miR-106a-5p, miR-150-5p, miR-125a-5p, miR-17-5p, miR-339-5p, miR-335-5p and miR-16-5p.

In some aspects, the classifiers disclosed herein are used prognostically. In some aspects, the classifiers disclosed herein are used predictively in a clinical setting, i.e., as predictive biomarkers. In some aspects, the classifiers disclosed herein can be used to stratify a population into different classes, e.g., for a clinical trial. In the context of the present disclosure, it is to be understood that the term classifier includes one or more classifiers, or combinations of classifiers, which can belong to the same or different classes (e.g., a score, e.g., Z-score, based classifier and a machine-learning based classifier, or several machine-learning based classifiers disclosed herein) wherein the term classifier is used to describe the output of a mathematical model assigning, e.g., a sample from a subject to a specific breast cancer class.

In some aspects, the classifier disclosed herein is a classifier obtained by the application of machine-learning techniques. In some specific aspects, the machine-learning technique is linear regression, e.g., Lasso regression. In some specific aspects, the machine learning technique is Random Forest. In some aspects, the machine-learning technique is selected from the group consisting of Linear Regression, Random Forest, Logistic Regression, Artificial Neural Network (ANN), Support Vector Machine (SVM), XGBoost (XGB), glmnet, cforest, Classification and Regression Trees for Machine-learning (CART), treebag, K-Nearest Neighbors (kNN), or a combination thereof.

The machine-learning classifiers generated by the machine-learning methods disclosed herein can be subsequently evaluated by determining the ability of the classifier to correctly call each test subject. In some aspects, the subjects of the training population used to derive the model are different from the subjects of the testing population used to test the model. As would be understood by a person skilled in the art, this allows one to predict the ability of the miRNA biomarker panel used to train the classifier as to their ability to properly characterize a subject whose breast cancer status is unknown.

The data which is inputted into the mathematical model can be any data which is representative of the expression level of the miRNA being evaluated. Mathematical models useful in accordance with the present disclosure include those using both supervised or unsupervised learning techniques. In some aspect of the disclosure, the mathematical model chosen uses supervised learning in conjunction with a “training population” to evaluate each of the possible combinations of miRNA biomarkers. In one aspect, the mathematical model used is selected from the following: a regression model, a logistic regression model, a neural network, a clustering model, principal component analysis, nearest-neighbor classifier analysis, linear discriminant analysis, quadratic discriminant analysis, a support vector machine, a decision tree, a genetic algorithm, classifier optimization using bagging, classifier optimization using boosting, classifier optimization using the Random Subspace Method, a projection pursuit, genetic programming and weighted voting. In some aspects, a logistic regression model is used. In other aspects, a decision tree model if used. In some aspects, a neural network model is used.

The results of applying a mathematical model of the present disclosure, e.g., a Lasso regression or Random forest model, to the data will generate one or more classifiers using one or more gene panels. In some aspects, multiple classifiers are created which are satisfactory for the given purpose (e.g., to correctly stage breast cancer). In this instance, in some aspects, a formula is generated which utilizes more than one classifier. For example, a formula can be generated which utilizes classifiers in series, e.g. a first classifier determiner the presence or absence of breast cancer, a second classifier determines the stage of the breast cancer, and a third classifier determines whether a particular treatment would be assigned to such breast cancer). In another aspect, a formula can be generated which results from weighting the results of more than one classifier. Other possible combinations and weightings of classifiers would be understood and are encompassed herein.

Classifiers (e.g., Lasso regression or Random forest models) generated according to the methods disclosed herein can be used to test an unknown or test subject. In one aspect, the model generated by a machine-learning method identified herein can detect whether an individual has breast cancer or a specific breast cancer stage. In some aspects, the model can predict whether a subject will respond to a particular therapy. In other aspects, the model can select or be used to select a subject for administration of a particular therapy.

In one aspect of the disclosure, each classifier is evaluated for its ability to properly characterize each subject of the training population using methods known to a person skilled in the art. For example, one can evaluate the classifier using cross validation, Leave One out Cross Validation (LOOCV), n-fold cross validation, or jackknife analysis using standard statistical methods. In another aspect of the present disclosure, each classifier is evaluated for its ability to properly characterize those subjects of the training population which were not used to generate the classifier.

In some aspects, one can train the classifier using one dataset, and evaluate the classifier on another distinct dataset. Accordingly, since the testing dataset is distinct from the training dataset, there is no need for cross validation.

In one aspect, the method used to evaluate the classifier for its ability to properly characterize each subject of the training population is a method that evaluates the classifier's sensitivity (TPF, true positive fraction) and 1-specificity (TNF, true negative fraction). In one aspect, the method used to test the classifier is Receiver Operating Characteristic (“ROC”) which provides several parameters to evaluate both the sensitivity and specificity of the result of the model generated, e.g., a model derived from the application of Lasso regression or Random forests.

In some aspects, the metrics used to evaluate the classifier for its ability to properly characterize each subject of the training population are classification accuracy (ACC), area under the receiver operating characteristic curve (AUC ROC), Sensitivity (true positive fraction, TPF), Specificity (true negative fraction, TNF), positive predicted value (PPV), negative predicted value (NPV), or any combination thereof. In one specific aspect, the metrics used to evaluate the classifier for its ability to properly characterize each subject of the training population are classification accuracy (ACC), area under the receiver operating characteristic curve (AUC ROC), Sensitivity (true positive fraction, TPF), Specificity (true negative fraction, TNF), positive predicted value (PPV), and negative predicted value (NPV).

In some aspects, the training set includes a reference population of at least about 10, at least about 20, at least about 30, at least about 40, at least about 50, at least about 60, at least about 70, at least about 80, at least about 90, at least about 100, at least about 110, at least about 120, at least about 130, at least about 140, at least about 150, at least about 160, at least about 170, at least about 180, at least about 190, at least about 200, at least about 250, at least about 300, at least about 350, at least about 400, at least about 450, at least about 500, at least about 600, at least about 700, at least about 800, at least about 900, or at least about 1000 subjects.

In some aspects, the training set includes a reference population of about 10, about 20, about 30, about 40, about 50, about 60, about 70, about 80, about 90, about 100, about 110, about 120, about 130, about 140, about 150, about 160, about 170, about 180, about 190, about 200, about 250, about 300, about 350, about 400, about 450, about 500, about 600, about 700, about 800, about 900, or about 1000 subjects.

In some aspects, the training set includes a reference population of between about 10 and about 20, about 20 and about 30, about 30 and about 40, about 40 and about 50, about 50 and about 60, about 60 and about 70, about 70 and about 80, about 80 and about 90, about 90 and about 100, about 100 and about 200, about 200 and about 300, about 300 and about 400, about 400 and about 500, about 500 and about 600, about 600, about 700, about 700 and about 800, about 800 and about 900, about 900 and about 1000 about 10 and about 200 subjects, about 200 and about 400, about 400 and about 600, about 600 and about 800, about 800 and about 1000, about 10 and about 250, about 250 and about 500, about 500 and about 750, or about 750 and about 1000 subject.

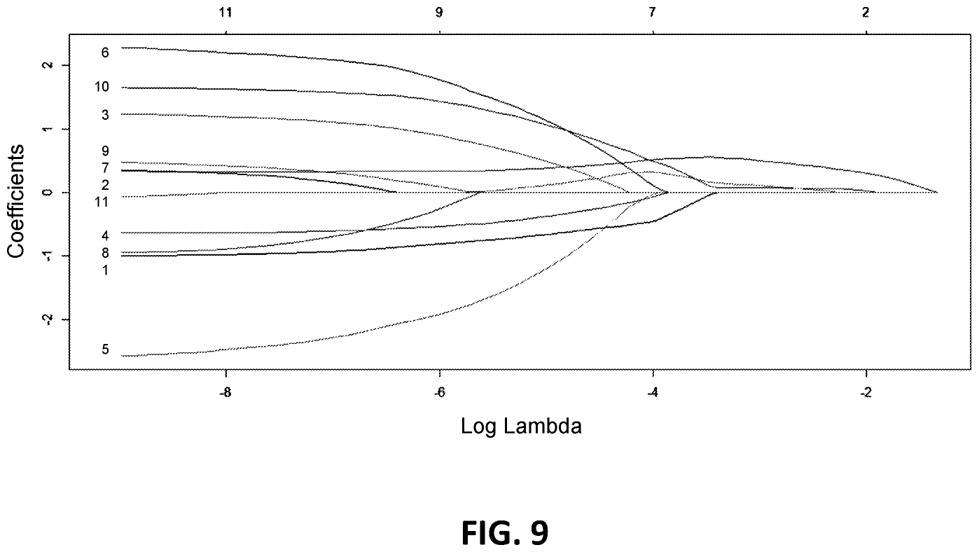

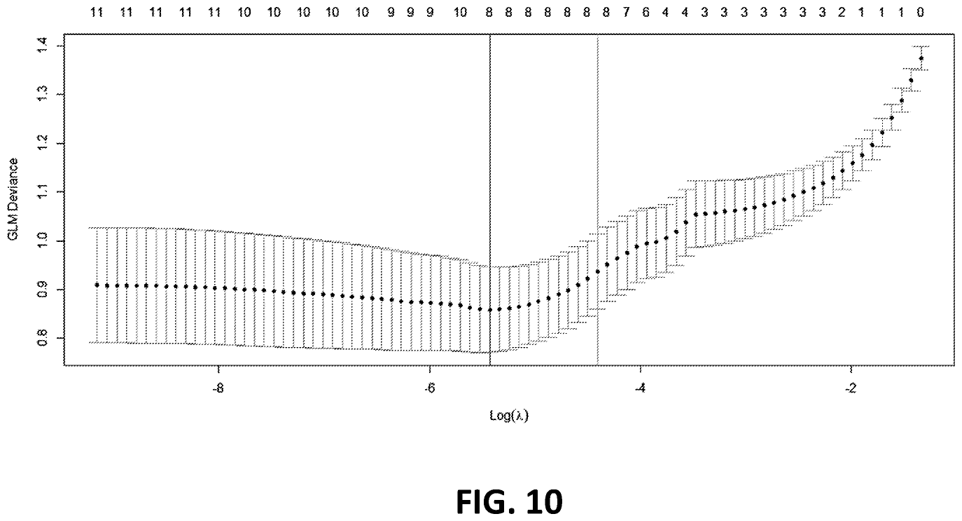

The Lasso Regression technique is based on a mathematical model that automatically penalizes those variables that are less relevant to the model or that do not provide new information, in order to eliminate them. This allows choosing those variables that have “survived” the selection technique objectively. The coefficient that Lasso uses to penalize is called Lambda (k), and as its value grows, the number of surviving variables decreases. To use this selection technique in the present work, the expression of the 11 candidate miRNAs transformed as mentioned above was used and, using the RStudio software, the analysis was carried out. As a result of the application of the algorithm, the software returns a series of graphs that account for the selection that was made. What is observed in FIG. 9 is, on the one hand, the representation of each of the 11 miRNAs with a determined color line. Then, the way in which each of the miRNAs is penalized is observed as the corresponding lines disappear as the X-axis is advanced from left to right (FIG. 9). In the upper part of the graph (FIG. 9), the numbers that are observed are the amount of miRNAs that were surviving in each point, and denote that, as the X axis advances, these numbers decrease. In the lower part of the graph (FIG. 9), the value of the Lambda logarithm is observed, and it is seen how, as it grows, the miRNAs disappear until finally all of them take a value of zero. Lasso regression then makes it possible to automatically define the optimal number of miRNAs to include in the model, and which miRNAs is it. In FIG. 10 it can be seen that 8 miRNAs were defined as the optimal number, a value that is defined in the upper part of the graph delimited by the horizontal lines. In particular, the 8 miRNAs selected by this method were: miR-150-5p, miR-106a-5p, miR-125a-5p, miR-17-5p, miR-339-5p, miR-339-3p, miR-335-5p and miR-16-5p.

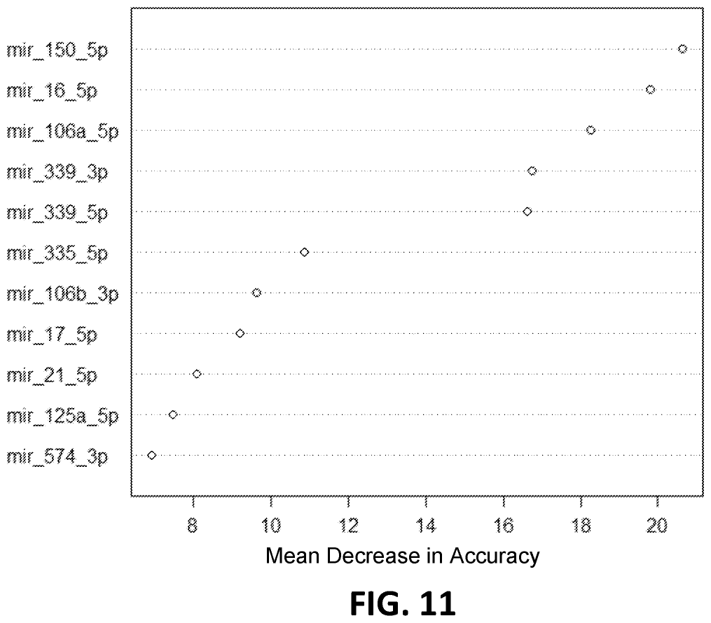

Another technique that was used in the selection of miRNAs to be used in predictive models was Random Forest. It consists of an algorithm that, through decision trees, results in a ranking of variables, from the most important to the least important according to this algorithm, while determining nodes or jumps in the importance of the variables, which makes the selection of variables clearer. In particular in this work, the ranking obtained by Random Forest is detailed in FIG. 11. In it, it can be seen how the miRNAs miR-150-5p, miR-16-5p, miR-106a-5p, miR-339-3p and miR-339-5p were classified in the ranking as the 5 most important according to the order of appearance and a jump was established between these and the following miRNAs, demonstrated with the change in the MeanDecreaseAccuracy value associated with each miRNA.

In some aspects, the methods disclosed herein comprise the use of a single predictive model (classifier) disclosed herein, e.g., Predictive Model 1 (PM1), Predictive Model 2 (PM2), Predictive Model 3 (PM3), Predictive Model 4 (PM4), or Predictive Model 5 (PM5). In some aspects, the methods disclosed herein comprise using PM1. In some aspects, the methods disclosed herein comprise using PM2. In some aspects, the methods disclosed herein comprise using PM3. In some aspects, the methods disclosed herein comprise using PM4. In some aspects, the methods disclosed herein comprise using PM5.

In some aspects, a method disclosed herein comprises using a single classifier, wherein the single classifier is PM1. In some aspects, a method disclosed herein comprises using a single classifier, wherein the single classifier is PM2. In some aspects, a method disclosed herein comprises using a single classifier, wherein the single classifier is PM3. In some aspects, a method disclosed herein comprises using a single classifier, wherein the single classifier is PM4. In some aspects, a method disclosed herein comprises using a single classifier, wherein the single classifier is PM5.

In some aspects, the methods disclosed herein comprise using two predictive models disclosed herein. In some aspects, the methods disclosed herein comprise using three predictive models disclosed herein. In some aspects, the methods disclosed herein comprise using four predictive models disclosed herein. In some aspects, the methods disclosed herein comprise using five predictive models disclosed herein.

In some aspects, the models (classifiers) disclosed herein used a statistical model called logistic regression associated with a cross validation of machine learning (Cross Validation Leave One Out). In some aspects, a value (p coefficient) is first obtained for each miRNA, which will then be informed within the equation to obtain a probability value. In some aspects, the probability value is compared with the threshold value or cut-off point, which will serve to classify the individual as healthy or sick. In other aspects, other statistical analysis techniques can be used.

Predictive Model 1—PM1: PM1 comprises analyzing the expression levels of a set of miRNAs consisting of miR-150-5p, miR-106b-3p, miR-106a-5p, miR-125a-5p, miR-17-5p, miR-574-3p, miR-339-5p, miR-339-3p, miR-335-5p, miR-16-5p and miR-21-5p. In some aspects, PM1 comprises
(i) averaging the Ct values obtained from the qPCR for each of the 11 specific miRNAs (miR-150-5p, miR-106b-3p, miR-106a-5p, miR-125a-5p, miR-17-5p, miR-574-3p, miR-339-5p, miR-339-3p, miR-335-5p, miR-16-5p and miR-21-5p);
(ii) subtracting the average value of each of the miRNAs from the Ct value obtained from the control (cel-miR-39-3p): Ct control−Ct miR_{specific}=ΔCtmiR_{specific};
(iii) squaring the ΔCtmiR_{specific}: 2\hat{ }(ΔCtmiR_{specific})=Value×miR_{specific};
(iv) calculating the logarithm in base e of Value×miR_{specific}: Ln(Value×miR_{specific})=Result miR_{specific}, which results in 4 values per individual: Value A (miR₁₅₀₋₅ₚ Result), Value B: (miR_{106b-3p} Result), Value C (miR₁₀₆ₐ₋₅ₚ Result), Value D (miR₁₂₅ₐ₋₅ₚ Result); Value E (miR₁₇₋₅ₚ Result), Value F (miR₅₇₄₋₃ₚ Result); Value G (miR₃₃₉₋₅ₚ Result), Value H (miR₃₃₉₋₃ₚ Result), Value I (miR₃₃₅₋₅ₚ Result); Value J (miR₁₆₋₅ₚ Result), Value K (miR₂₁₋₅ₚ Result);
(v) calculating the probability of having breast cancer for each individual by integrating the 11 results of the miRNAs in the following equation:

$$p ( x ) = 1 / ( e \hat{} ( - ( {β}_{0} + {β}_{1} * ValueA + {β}_{2} * ValueB + {β}_{3} * ValueC + {β}_{4} * ValueD + {β}_{5} * ValueE + {β}_{6} * ValueF + {β}_{7} * ValueG + {β}_{8} * ValueH + {β}_{9} * ValueI + {β}_{10} * ValueJ + {β}_{11} * ValueK ) ) + 1 ) ,$$

wherein the values of the beta coefficients are the following: β₀=2.6258, β₁=0.3280, β₂=−0.990, β₃=1.2630, ₄=−0.6357, β₅=−2.6589, β₆=0.5139, β₇=−0.1197, β₈=2.3412, β₉=−1.0167, β₁₀=1.6683, β₁₁=0.3948
(vi) comparing the obtained probability score (p(x)) with the specific cut-off point for this predictive model, which is 0.39;
wherein if the value of the individual's probability of having breast cancer is equal to or greater than 0.39, the individual will be classified as sick, and if it is less than 0.39, it will be classified as healthy.

In some aspects, PM1 comprises calculating the probability of having breast cancer for each individual by integrating the 11 results of the miRNAs in the following equation:

$$p ( x ) = 1 / ( e \hat{} ( - ( {β}_{0} + {β}_{1} * ValueA + {β}_{2} * ValueB + {β}_{3} * ValueC + {β}_{4} * ValueD + {β}_{5} * ValueE + {β}_{6} * ValueF + {β}_{7} * ValueG + {β}_{8} * ValueH + {β}_{9} * ValueI + {β}_{10} * ValueJ + {β}_{11} * ValueK ) ) + 1 ) ,$$

wherein the values of the beta coefficients are the following: β₀=2.6258, β₁=0.3280, β₂=−0.990, β₃=1.2630, ₄=−0.6357, β₅=−2.6589, β₆=0.5139, β₇=−0.1197, β₈=2.3412, β₉=−1.0167, β₁₀=1.6683, β₁₁=0.3948, and wherein each parameter in the equation has been calculated as described above. In some aspects, PM1 further comprises comparing the obtained probability score (p(x)) with the specific cut-off point for this predictive model, which is 0.39; wherein if the value of the individual's probability of having breast cancer is equal to or greater than 0.39, the individual will be classified as sick, and if it is less than 0.39, it will be classified as healthy.

In some aspects, PM1 has a sensitivity of about 87%. In some aspects, PM1 has a specificity of about 73%. In some aspects, PM1 has an AUCROC of about 0.88. In some aspects, PM1 has an accuracy of about 81%. In some aspects, PM1 has a positive predictive value of about 81%. In some aspects, PM1 has a negative predictive value of about 80%. In some aspects, PM1 has a false positive rate of about 23%.

Predictive Model 2—PM2: PM2 comprises analyzing the expression levels of a set of miRNAs consisting of miR106a-5p, miR17-5p, miR339-3p, miR335-5p, and miR16-5p. In some aspects, PM2 comprises
(i) averaging the Ct values obtained from the qPCR for each of the 5 specific miRNAs Averaging the Ct values obtained from the qPCR for each miRNA separately (miR106a-5p, miR17-5p, miR339-3p, miR16-5p, miR335-5p);
(ii) subtracting the average value of each of the miRNAs from the Ct value obtained from the control (cel-miR-39-3p): Ct control−Ct miR_{specific}=ΔCtmiR_{specific};
(iii) squaring the ΔCtmiR_{specific}: 2\hat{ }(ΔCtmiR_{specific})=Value×miR_{specific};
(iv) calculating the logarithm in base e of Value×miR_{specific}: Ln(Value×miR_{specific})=Result miR_{specific},
which results in 5 values per individual: Value A (miR₁₀₆ₐ₋₅ₚ Result), Value B (miR₁₇₋₅ₚ Result), Value C (miR₃₃₉₋₃ₚ Result), Value D (miR₁₆₋₅ₚ Result), Value E (miR₃₃₅₋₅ₚ Result);
(v) calculating the probability of having breast cancer for each individual by integrating the 5 results of the miRNAs in the following equation:

p(x)=1/(e\hat{ }(−(β₀+β₁*Value A+β₂*Value B+β₃*Value C+β₄*Value D+β₅*Value E))+1), wherein the values of the beta coefficients are the following: β₀=3.9420,β₁=1.0664,β₂=−2.8282,β₃=1.8165,β₄=1.9203,β₅=−0.824; and
(vi) comparing the obtained probability score (p(x)) with the specific cut-off point for this predictive model, which is 0.4432;
wherein if the value of the individual's probability of having breast cancer is equal to or greater than 0.39, the individual will be classified as sick, and if it is less than 0.4432, it will be classified as healthy.

In some aspects, PM2 comprises calculating the probability of having breast cancer for each individual by integrating the 5 results of the miRNAs in the following equation: p(x)=1/(e\hat{ }(−(β₀+β₁*Value A+β₂*Value B+β₃*Value C+β₄*Value D+β₅*Value E))+1), wherein the values of the beta coefficients are the following: β₀=3.9420, β₁=1.0664, β₂=−2.8282, β₃=1.8165, β₄=1.9203, β₅=−0.824, and wherein each parameter in the equation has been calculated as described above. In some aspects, PM2 further comprises comparing the obtained probability score (p(x)) with the specific cut-off point for this predictive model, which is 0.4432; wherein if the value of the individual's probability of having breast cancer is equal to or greater than 0.39, the individual will be classified as sick, and if it is less than 0.4432, it will be classified as healthy.

In some aspects, PM2 has a sensitivity of about 88%. In some aspects, PM2 has a specificity of about 77%. In some aspects, PM2 has an AUCROC of about 0.89. In some aspects, PM2 has an accuracy of about 83%. In some aspects, PM2 has a positive predictive value of about 84%. In some aspects, PM2 has a negative predictive value of about 82%. In some aspects, PM2 has a false positive rate of about 23%.

Predictive Model 3—PM3: PM3 comprises analyzing the expression levels of a set of miRNAs consisting of miR105-5p, miR106a-5p, miR125a-5p, miR17-5p, miR339-5p, miR339-3p, miR335-5p, and miR26-5p. In some aspects, PM3 comprises
(i) averaging the Ct values obtained from the qPCR for each of the 8 specific miRNAs Averaging the Ct values obtained from the qPCR for each miRNA separately (miR150-5p, miR106a-5p, miR125a-5p, miR17-5p, miR339-5p, miR339-3p, miR335-5p, miR16-5p);
(ii) subtracting the average value of each of the miRNAs from the Ct value obtained from the control (cel-miR-39-3p): Ct control−Ct miR_{specific}=ΔCtmiR_{specific};
(iii) squaring the ΔCtmiR_{specific}: 2\hat{ }(ΔCtmiR_{specific})=Value×miR_{specific};
(iv) calculating the logarithm in base e of Value×miR_{specific}: Ln(Value×miR_{specific})=Result miR_{specific},
which results in 8 values per individual: Value A (miR₁₅₀₋₅ₚ Result), Value B (miR₁₀₆ₐ₋₅ₚ Result), Value C (miR₁₂₅ₐ₋₅ₚ Result), Value D (miR₁₇₋₅ₚ Result), Value E (miR₃₃₉₋₅ₚ Result), Value F (miR₃₃₉₋₃ₚ Result), Value G (miR₃₃₅₋₅ₚ Result), Value H (miR₁₆₋₅ₚ Result);
(v) calculating the probability of having breast cancer for each individual by integrating the 8 results of the miRNAs in the following equation: p(x)=1/(e\hat{ }(−(β₀+β₁*Value A+β₂*Value B+β₃*Value C+β₄*Value D+β₅*Value E+β₆*Value F+β₇*Value G+β₈*Value H))+1),
wherein the values of the beta coefficients are the following:
β₀=4.0444, β₁=0.2895, β₂=1.1846, β₃=−0.6834, β₄=−2.5341, β₅=−0.1790, β₆=2.2036, β₇=−0.9407, β₈=1.7400;
(vi) comparing the obtained probability score (p(x)) with the specific cut-off point for this predictive model, which is 0.6217;
wherein if the value of the individual's probability of having breast cancer is equal to or greater than 0.6217, the individual will be classified as sick, and if it is less than 0.6217, it will be classified as healthy.

In some aspects, PM3 comprises calculating the probability of having breast cancer for each individual by integrating the 8 results of the miRNAs in the following equation: p(x)=1/(e\hat{ }(−(β₀+β₁*Value A+β₂*Value B+β₃*Value C+β₄*Value D+β₅*Value E+β₆*Value F+β₇*Value G+β₈*Value H))+1), wherein the values of the beta coefficients are the following; β₀=4.0444, β₁=0.2895, β₂=1.1846, β₃=−0.6834, β₄=−2.5341, β₅=−0.1790, β₆=2.2036, β₇=−0.9407, β₈=1.7400, and wherein each parameter in the equation has been calculated as described above. In some aspects, PM3 further comprises comparing the obtained probability score (p(x)) with the specific cut-off point for this predictive model, which is 0.6217.

In some aspects, PM3 has a sensitivity of about 77%. In some aspects, PM3 has a specificity of about 86%. In some aspects, PM3 has an AUCROC of about 0.89. In some aspects, PM3 has an accuracy of about 81%. In some aspects, PM3 has a positive predictive value of about 89%. In some aspects, PM3 has a negative predictive value of about 73%. In some aspects, PM3 has a false positive rate of about 14%.

Predictive Model 4—PM4: PM4 comprises analyzing the expression levels of a set of miRNA consisting of miR106-5p, miR17-5p, miR339-3p, and miR16-5p. In some aspects, PM4 comprises
(i) averaging the Ct values obtained from the qPCR for each of the 4 specific miRNAs (miR-106a-5p, miR-17-5p, miR-339-3p and miR-16-5p);
(ii) subtracting the average value of each of the miRNAs from the Ct value obtained from the control (cel-miR-39-3p): Ct control−Ct miR_{specific}=ΔCtmiR_{specific};
(iii) squaring the ΔCtmiR_{specific}: 2\hat{ }(ΔCtmiR_{specific})=Value×miR_{specific};
(iv) calculating the logarithm in base e of Value×miR_{specific}:

$$Ln ( Value \times {miR}_{specific} ) = Result {miR}_{specific ,}$$

which results in 4 values per individual: Value A (miR₁₀₆ₐ₋₅ₚ Result), Value B (miR₁₇₋₅ₚ Result), Value C (miR₃₃₉₋₃ₚ Result) and Value D (miR₁₆₋₅ₚ Result);
(v) calculating the probability of having breast cancer for each individual by integrating the 4 results of the miRNAs in the following equation:

$$p ( x ) = 1 / ( e \hat{} ( - ( {β}_{0} + {β}_{1} * Value A + {β}_{2} * Value B + {β}_{3} * Value C + {β}_{4} * Value D ) ) + 1 )$$

wherein the values of the beta coefficients are:
β₀=5.9446; β₁=1.1062; β₂=−3.5628; β₃=1.5886; β₄=1.9661; and,
(vi) comparing the obtained probability score (p(x)) with the specific cut-off point for this predictive model, which is 0.3744;
wherein if the value of the individual's probability of having breast cancer is equal to or greater than 0.3744, the individual will be classified as sick, and if it is less than 0.3744, it will be classified as healthy.

In some aspects, PM4 comprises calculating the probability of having breast cancer for each individual by integrating the 4 results of the miRNAs in the following equation: p(x)=1/(e\hat{ }(−(β₀+β₁*Value A+β₂*Value B+β₃*Value C+β₄*Value D))+1) wherein the values of the beta coefficients are: β₀=5.9446; β₁=1.1062; β₂=−3.5628; β₃=1.5886; β₄=1.9661; and, wherein each parameter in the equation has been calculated as described above. In some aspects, PM4 further comprises comparing the obtained probability score (p(x)) with the specific cut-off point for this predictive model, which is 0.3744; wherein if the value of the individual's probability of having breast cancer is equal to or greater than 0.3744, the individual will be classified as sick, and if it is less than 0.3744, it will be classified as healthy.

In some aspects, PM4 has a sensitivity of about 92%. In some aspects, PM4 has a specificity of about 71%. In some aspects, PM4 has an AUCROC of about 0.89. In some aspects, PM4 has an accuracy of about 83%. In some aspects, PM4 has a positive predictive value of about 81%. In some aspects, PM4 has a negative predictive value of about 87%. In some aspects, PM4 has a false positive rate of about 29%.

Predictive Model 5—PM5: PM5 comprises analyzing the expression levels of a set of miRNA consisting of miR150-5p, miR106-5p, miR339-5p, miR339-3p, and miR16-5p. In some aspects, PM5 comprises
(i) averaging the Ct values obtained from the qPCR for each of the 5 specific miRNAs Averaging the Ct values obtained from the qPCR for each miRNA separately (miR150-5p, miR106a-5p, miR339-5p, miR339-3p, miR16-5);
(ii) subtracting the average value of each of the miRNAs from the Ct value obtained from the control (cel-miR-39-3p): Ct control−Ct miR_{specific}=ΔCtmiR_{specific};
(iii) squaring the ΔCtmiR_{specific}: 2\hat{ }(ΔCtmiR_{specific})=Value×miR_{specific};
(iv) calculating the logarithm in base e of Value×miR_{specific}: Ln(Value×miR_{specific})=Result miR_{specific},
which results in 5 values per individual: Value A (miR₁₅₀₋₅ₚ Result), Value B (miR₁₀₆ₐ₋₅ₚ Result), Value C (miR₃₃₉₋₅ₚ Result), Value D (miR₃₃₉₋₃ₚ Result), Value E (miR₁₆₋₅ Result);
(v) calculating the probability of having breast cancer for each individual by integrating the 5 results of the miRNAs in the following equation: p(x)=1/(e\hat{ }(−(β₀+β₁*Value A+β₂*Value B+β₃*Value C+β₄*Value D+β₅*Value E))+1), wherein the values of the beta coefficients are the following: β₀=2.8464, β₁=0.6173, β₂=−0.3038, β₃=0.5280, β₄=−0.3079, β₅=0.4969
(vi) comparing the obtained probability score (p(x)) with the specific cut-off point for this predictive model, which is 0.4905;
wherein if the value of the individual's probability of having breast cancer is equal to or greater than 0.4905, the individual will be classified as sick, and if it is less than 0.4905, it will be classified as healthy.

In some aspects, PM5 comprises calculating the probability of having breast cancer for each individual by integrating the 5 results of the miRNAs in the following equation: p(x)=1/(e\hat{ }(−(β₀+β₁*Value A+β₂*Value B+β₃*Value C+β₄*Value D+β₅*Value E))+1), wherein the values of the beta coefficients are the following: β₀=2.8464, β₁=0.6173, β₂=−0.3038, β₃=0.5280, β₄=−0.3079, β₅=0.4969, and wherein each parameter in the equation has been calculated as described above. In some aspects, PM5 further comprises comparing the obtained probability score (p(x)) with the specific cut-off point for this predictive model, which is 0.4905; wherein if the value of the individual's probability of having breast cancer is equal to or greater than 0.4905, the individual will be classified as sick, and if it is less than 0.4905, it will be classified as healthy.

In some aspects, PM5 has a sensitivity of about 85%. In some aspects, PM5 has a specificity of about 66%. In some aspects, PM5 has an AUCROC of about 0.8. In some aspects, PM5 has an accuracy of about 77%. In some aspects, PM5 has a positive predictive value of about 77%. In some aspects, PM5 has a negative predictive value of about 76%. In some aspects, PM5 has a false positive rate of about 34%.

Predictive Model 6—PM6: PM6 comprises analyzing the expression levels of a set of miRNAs consisting of miR-150-5p, miR-106b-3p, miR-106a-5p, miR-125a-5p, miR-17-5p, miR-574-3p, miR-339-5p, miR-335-5p, miR-16-5p and miR-21-5p. In some aspects, PM6 comprises
(i) averaging the Ct values obtained from the qPCR for each of the 10 specific miRNAs (miR-150-5p, miR-106b-3p, miR-106a-5p, miR-125a-5p, miR-17-5p, miR-574-3p, miR-339-5p, miR-335-5p, miR-16-5p and miR-21-5p);
(ii) subtracting the average value of each of the miRNAs from the Ct value obtained from the control (cel-miR-39-3p): Ct control−Ct miR_{specific}=ΔCtmiR_{specific};
(iii) squaring the ΔCtmiR_{specific}: 2\hat{ }(ΔCtmiR_{specific})=Value×miR_{specific};
(iv) calculating the logarithm in base e of Value×miR_{specific}: Ln(Value×miR_{specific})=Result miR_{specific}, which results in 4 values per individual: Value A (miR₁₅₀₋₅ₚ Result), Value B: (miR_{106b-3p} Result), Value C (miR₁₀₆ₐ₋₅ₚ Result), Value D (miR₁₂₅ₐ₋₅ₚ Result); Value E (miR₁₇₋₅ₚ Result), Value F (miR₅₇₄₋₃ₚ Result); Value G (miR₃₃₉₋₅ₚ Result), Value H (miR₃₃₉₋₃ₚ Result), Value I (miR₃₃₅₋₅ₚ Result); Value J (miR₁₆₋₅ₚ Result), Value K (miR₂₁₋₅ₚ Result);
(v) calculating the probability of having breast cancer for each individual by integrating the 10 results of the miRNAs in the following equation:

$$p ( x ) = 1 / ( e \hat{} ( - ( {β}_{0} + {β}_{1} * ValueA + {β}_{2} * ValueB + {β}_{3} * ValueC + {β}_{4} * ValueD + {β}_{5} * ValueE + {β}_{6} * ValueF + {β}_{7} * ValueG + {β}_{8} * ValueH + {β}_{9} * ValueI + {β}_{10} * ValueJ + {β}_{11} * ValueK ) ) + 1 ) ,$$

wherein the values of the beta coefficients are the following: β₀=2.6258, β₁=0.3280, β₂=−0.990, β₃=1.2630, ₄=−0.6357, β₅=−2.6589, β₆=0.5139, β₇=−0.1197, β₈=2.3412, β₉=−1.0167, β₁₀=1.6683, β₁₁=0.3948
(vi) comparing the obtained probability score (p(x)) with the specific cut-off point for this predictive model, which is 0.39;
wherein if the value of the individual's probability of having breast cancer is equal to or greater than 0.39, the individual will be classified as sick, and if it is less than 0.39, it will be classified as healthy.

In some aspects, PM1 comprises calculating the probability of having breast cancer for each individual by integrating the 10 results of the miRNAs in the following equation:

$$p ( x ) = 1 / ( e \hat{} ( - ( {β}_{0} + {β}_{1} * ValueA + {β}_{2} * ValueB + {β}_{3} * ValueC + {β}_{4} * ValueD + {β}_{5} * ValueE + {β}_{6} * ValueF + {β}_{7} * ValueG + {β}_{8} * ValueH + {β}_{9} * ValueI + {β}_{10} * ValueJ + {β}_{11} * ValueK ) ) + 1 ) ,$$

wherein the values of the beta coefficients are the following: β₀=2.6258, β₁=0.3280, β₂=−0.990, β₃=1.2630, ₄=−0.6357, β₅=−2.6589, β₆=0.5139, β₇=−0.1197, β₈=2.3412, β₉=−1.0167, β₁₀=1.6683, β₁₁=0.3948, and wherein each parameter in the equation has been calculated as described above. In some aspects, PM6 further comprises comparing the obtained probability score (p(x)) with the specific cut-off point for this predictive model, which is 0.39; wherein if the value of the individual's probability of having breast cancer is equal to or greater than 0.39, the individual will be classified as sick, and if it is less than 0.39, it will be classified as healthy.

In some aspects, PM6 has a sensitivity of about 87%. In some aspects, PM6 has a specificity of about 73%. In some aspects, PM6 has an AUCROC of about 0.88. In some aspects, PM6 has an accuracy of about 81%. In some aspects, PM1 has a positive predictive value of about 81%. In some aspects, PM1 has a negative predictive value of about 80%. In some aspects, PM6 has a false positive rate of about 23%.

Predictive Model 7—PM7: PM7 comprises analyzing the expression levels of a set of miRNA consisting of miR-106a-5p, miR-125a-5p, miR-150-5p, miR-17-5p, miR-339-5p, miR-335-5p and miR-16-5p. In some aspects, PM7 comprises
(i) averaging the Ct values obtained from the qPCR for each of the 7 specific miRNAs Averaging the Ct values obtained from the qPCR for each miRNA separately miR-106a-5p, miR-125a-5p, miR-150-5p, miR-17-5p, miR-339-5p, miR-335-5p and miR-16-5p);
(ii) subtracting the average value of each of the miRNAs from the Ct value obtained from the control (cel-miR-39-3p): Ct control−Ct miR_{specific}=ΔCtmiR_{specific};
(iii) squaring the ΔCtmiR_{specific}: 2\hat{ }(ΔCtmiR_{specific})=Value×miR_{specific};
(iv) calculating the logarithm in base e of Value×miR_{specific}: Ln(Value×miR_{specific})=Result miR_{specific},
which results in 5 values per individual: Value A (miR₁₅₀₋₅ₚ Result), Value B (miR₁₀₆ₐ₋₅ₚ Result), Value C (miR₃₃₉₋₅ₚ Result), Value D (miR₃₃₉₋₃ₚ Result), Value E (miR₁₆₋₅ Result);
(v) calculating the probability of having breast cancer for each individual by integrating the 5 results of the miRNAs in the following equation: p(x)=1/(e\hat{ }(−(β₀+β₁*Value A+β₂*Value B+β₃*Value C+β₄*Value D+β₅*Value E))+1), wherein the values of the beta coefficients are the following: β₀=2.8464, β₁=0.6173, β₂=−0.3038, β₃=0.5280, β₄=−0.3079, β₅=0.4969
(vi) comparing the obtained probability score (p(x)) with the specific cut-off point for this predictive model, which is 0.4905;
wherein if the value of the individual's probability of having breast cancer is equal to or greater than 0.4905, the individual will be classified as sick, and if it is less than 0.4905, it will be classified as healthy.

In some aspects, PM7 comprises calculating the probability of having breast cancer for each individual by integrating the 5 results of the miRNAs in the following equation: p(x)=1/(e\hat{ }(−(β₀+β₁*Value A+β₂*Value B+β₃*Value C+β₄*Value D+β₅*Value E))+1), wherein the values of the beta coefficients are the following: β₀=2.8464, β₁=0.6173, β₂=−0.3038, β₃=0.5280, β₄=−0.3079, β₅=0.4969, and wherein each parameter in the equation has been calculated as described above. In some aspects, PM7 further comprises comparing the obtained probability score (p(x)) with the specific cut-off point for this predictive model, which is 0.4905; wherein if the value of the individual's probability of having breast cancer is equal to or greater than 0.4905, the individual will be classified as sick, and if it is less than 0.4905, it will be classified as healthy.

In some aspects, PM7 has a sensitivity of about 85%. In some aspects, PM7 has a specificity of about 66%. In some aspects, PM7 has an AUCROC of about 0.8. In some aspects, PM7 has an accuracy of about 77%. In some aspects, PM7 has a positive predictive value of about 77%. In some aspects, PM7 has a negative predictive value of about 76%. In some aspects, PM7 has a false positive rate of about 34%.

Methods of treatment: The present disclosure provides methods (e.g., PM1, PM2, PM3, PM4, PM5, PM6, PM7 or combinations thereof) for classifying/stratifying patients and/or cancer samples from those patients according to a breast cancer class assignment (e.g., absence or presence, or stage) resulting from applying a classifier derived from a combined biomarker (e.g., a set of miRNA expression data corresponding to a miRNA biomarker panel of the present disclosure). In some aspects, the classifier is a machine-learning based classifier, e.g., a Lasso regression model or a Random forest model disclosed herein, or a combination thereof. Based on the identification of a specific breast cancer status, a specific therapy can be selected to treat the patient's breast cancer.

In one aspect, the present disclosure provides a method for treating a human subject afflicted with breast can comprising administering a breast cancer therapy to the subject wherein, prior to the administration, the subject is identified via a classifier of the present disclosure as having breast cancer.

The present disclosure also provides a method for treating a human subject afflicted with breast cancer comprising (a) identifying a subject having breast cancer via a classifier, e.g., machine-learning classifier disclosed herein, and (b) administering a breast cancer therapy to the subject.

Also provided is a method for identifying a human subject afflict with a breast cancer suitable for treatment with a specific breast cancer therapy, the method comprising determining the presence of breast cancer in the subject via a classifier, e.g., a machine-learning classifier disclosed herein, as determined by measuring the miRNA expression levels of a plurality of miRNA biomarkers of the present disclosure, e.g., a full panel comprising, consisting, or consisting essentially of miR-150-5p, miR-106b-3p, miR-106a-5p, miR-125a-5p, miR-17-5p, miR-574-3p, miR-339-5p, miR-339-3p, miR-335-5p, miR-16-5p and miR-21-5p, or a biomarker panel comprising, consisting, or consisting essentially of a subset thereof, e.g., miR-16-5p, miR-17-5p, miR-106a-5p, and miR-339-3p; miR-106a-5p, miR-17-5p, miR-339-3p, miR-16-5p and miR-335-5p; miR-106a-5p, miR-17-5p, miR-339-3p, miR-16-5p, miR-150-5p, miR-125a-5p, miR-339-5p, and miR-335-5p; miR-150-5p, miR-106b-3p, miR-106a-5p, miR-125a-5p, miR-17-5p, miR-574-3p, miR-339-5p, miR-339-3p, miR-335-5p, miR-16-5p and miR-21-5p; miR-150-5p, miR-106b-3p, miR-106a-5p, miR-125a-5p, miR-17-5p, miR-574-3p, miR-339-5p, miR-335-5p, miR-16-5p and miR-21-5p; or, miR-106a-5p, miR-150-5p, miR-125a-5p, miR-17-5p, miR-339-5p, miR-335-5p and miR-16-5p; wherein the identification of the subject as having breast cancer indicates that a breast cancer therapy can be administered to treat the cancer.

The present disclosure also provides a gene panel comprising, consisting, or consisting essentially of miR-150-5p, miR-106b-3p, miR-106a-5p, miR-125a-5p, miR-17-5p, miR-574-3p, miR-339-5p, miR-339-3p, miR-335-5p, miR-16-5p and miR-21-5p, or a biomarker panel comprising, consisting, or consisting essentially of a subset thereof, e.g., miR-16-5p, miR-17-5p, miR-106a-5p, and miR-339-3p; miR-106a-5p, miR-17-5p, miR-339-3p, miR-16-5p and miR-335-5p; miR-106a-5p, miR-17-5p, miR-339-3p, miR-16-5p, miR-150-5p, miR-125a-5p, miR-339-5p, and miR-335-5p; miR-150-5p, miR-106b-3p, miR-106a-5p, miR-125a-5p, miR-17-5p, miR-574-3p, miR-339-5p, miR-339-3p, miR-335-5p, miR-16-5p and miR-21-5p; miR-150-5p, miR-106b-3p, miR-106a-5p, miR-125a-5p, miR-17-5p, miR-574-3p, miR-339-5p, miR-335-5p, miR-16-5p and miR-21-5p; or, miR-106a-5p, miR-150-5p, miR-125a-5p, miR-17-5p, miR-339-5p, miR-335-5p and miR-16-5p for use in determining the breast cancer status (e.g., presence or absence of breast cancer, of breast cancer stage) in a subject in need thereof via a classifier or combination thereof disclosed herein, e.g., a Lasso regression or Random forest classifier, wherein the breast cancer status is used for (i) identifying a subject suitable for an anticancer therapy; (ii) determining the prognosis of a subject undergoing anticancer therapy; (iii) initiating, suspending, or modifying the administration of an anticancer therapy; or, (iv) a combination thereof. In some aspects, the miRNA biomarker panel is used according to the methods disclosed here, e.g., to classify a breast cancer from a patient (e.g., for staging) and to administer a specific therapy (e.g., a breast cancer therapy disclosed herein or a combination thereof) based on that classification.

### Additional Methods of Detection

The present disclosure provides an in vitro method for the detection of breast cancer, comprising:
(i) obtaining a biological sample from an individual in whom it is desired to determine if they have breast cancer; for example, a biological sample comprising venous blood; and,
(ii) determine the expression level of at least four miRNAs selected from the set comprised by miR-150-5p, miR-106b-3p, miR-106a-5p, miR-125a-5p, miR-17-5p, miR-574-3p, miR-339-5p, miR-339-3p, miR-335-5p, miR-16-5p, and miR-21-5p; where an increase in the expression of said at least four miRNAs compared to a control, is indicative of the presence of breast cancer. In some aspects, said group of miRNAs is selected from the set comprised by miR-106a-5p, miR-17-5p, miR-339-3p, miR-16-5p, miR-150-5p, miR-125a-5p, miR-339-5p, and miR-335-5p. In some aspects, said group of miRNAs is selected from the set comprised by miR-106a-5p, miR-339-3p, miR-16-5p, miR-150-5p and miR-339-5p. In some aspects, said group of miRNAs is selected from the set comprised by miR-106a-5p, miR-17-5p, miR-339-3p, miR-16-5p and miR-335-5p. In some aspects, said miRNAs comprise miR-106a-5p, miR-17-5p, miR-339-3p, and miR-16-5p.

The present disclosure also provides an in vitro method for the detection of breast cancer, which comprises:
(i) obtaining a biological sample from a patient; for example, a biological sample comprising venous blood; and.
(ii) determining the expression level of at least eight miRNAs selected from the set comprised by miR-150-5p, miR-106b-3p, miR-106a-5p, miR-125a-5p, miR-17-5p, miR-574-3p, miR-339-5p, miR-339-3p, miR-335-5p, miR-16-5p, and miR-21-5p; where an increase in the expression of said at least four miRNAs compared to a control, is indicative of risk of breast cancer. In some aspects, said miRNAs comprise miR-106a-5p, miR-17-5p, miR-339-3p, miR-16-5p, miR-150-5p, miR-125a-5p, miR-339-5p, and miR-335-5p.

The present disclosure also provides an in vitro method for the detection of breast cancer, which comprises:
(i) obtaining a biological sample from a patient; for example, a biological sample comprising venous blood; and.
(ii) determining the expression level of at least seven or ten miRNAs selected from the set comprised by miR-150-5p, miR-106b-3p, miR-106a-5p, miR-125a-5p, miR-17-5p, miR-574-3p, miR-339-5p, miR-339-3p, miR-335-5p, miR-16-5p, and miR-21-5p; where an increase in the expression of said at least seven or ten miRNAs compared to a control, is indicative of risk of breast cancer. In some aspects, said set of seven miRNAs comprises, consists, or consists essentially of miR-106a-5p, miR-150-5p, miR-125a-5p, miR-17-5p, miR-339-5p, miR-335-5p and miR-16-5p. In some aspects, said set of ten miRNAs comprises, consists, or consists essentially of miR-150-5p, miR-106b-3p, miR-106a-5p, miR-125a-5p, miR-17-5p, miR-574-3p, miR-339-5p, miR-335-5p, miR-16-5p. and miR-21-5p.

The present disclosure also provides an in vitro method for the detection of breast cancer, which comprises:
(i) obtaining a biological sample from a patient; and,
(ii) determining the expression level of the set comprised of miR-150-5p, miR-106b-3p, miR-106a-5p, miR-125a-5p, miR-17-5p, miR-574-3p, miR-339-5p, miR-339-3p, miR-335-5p, miR-16-5p, and miR-21-5p; where an increase in the expression of said miRNAs compared to a control, is indicative of risk of breast cancer.

In some aspects, the term “administering” can also comprise commencing a therapy, discontinuing or suspending a therapy, temporarily suspending a therapy, or modifying a therapy (e.g., increasing dosage or frequency of doses, or adding one of more therapeutic agents in a combination therapy).

In some aspects, samples can, for example, be requested by a healthcare provider (e.g., a doctor) or healthcare benefits provider, obtained and/or processed by the same or a different healthcare provider (e.g., a nurse, a hospital) or a clinical laboratory, and after processing, the results can be forwarded to the original healthcare provider or yet another healthcare provider, healthcare benefits provider or the patient. Similarly, the quantification of the expression level of a biomarker disclosed herein; comparisons between biomarker scores or protein expression levels; evaluation of the absence or presence of biomarkers; determination of biomarker levels with respect to a certain threshold; treatment decisions; or combinations thereof, can be performed by one or more healthcare providers, healthcare benefits providers, and/or clinical laboratories.

As used herein, the term “healthcare provider” refers to individuals or institutions that directly interact with and administer to living subjects, e.g., human patients. Non-limiting examples of healthcare providers include doctors, nurses, technicians, therapist, pharmacists, counselors, alternative medicine practitioners, medical facilities, doctor's offices, hospitals, emergency rooms, clinics, urgent care centers, alternative medicine clinics/facilities, and any other entity providing general and/or specialized treatment, assessment, maintenance, therapy, medication, and/or advice relating to all, or any portion of, a patient's state of health, including but not limited to general medical, specialized medical, surgical, and/or any other type of treatment, assessment, maintenance, therapy, medication and/or advice.

As used herein, the term “clinical laboratory” refers to a facility for the examination or processing of materials derived from a living subject, e.g., a human being. Non-limiting examples of processing include biological, biochemical, serological, chemical, immunohematological, hematological, biophysical, cytological, pathological, genetic, or other examination of materials derived from the human body for the purpose of providing information, e.g., for the diagnosis, prevention, or treatment of any disease or impairment of, or the assessment of the health of living subjects, e.g., human beings. These examinations can also include procedures to collect or otherwise obtain a sample, prepare, determine, measure, or otherwise describe the presence or absence of various substances in the body of a living subject, e.g., a human being, or a sample obtained from the body of a living subject, e.g., a human being.

As used herein, the term “healthcare benefits provider” encompasses individual parties, organizations, or groups providing, presenting, offering, paying for in whole or in part, or being otherwise associated with giving a patient access to one or more healthcare benefits, benefit plans, health insurance, and/or healthcare expense account programs.

In some aspects, a healthcare provider can administer or instruct another healthcare provider to administer a therapy disclosed herein to treat a cancer. A healthcare provider can implement or instruct another healthcare provider or patient to perform the following actions: obtain a sample, process a sample, submit a sample, receive a sample, transfer a sample, analyze or measure a sample, quantify a sample, provide the results obtained after analyzing/measuring/quantifying a sample, receive the results obtained after analyzing/measuring/quantifying a sample, compare/score the results obtained after analyzing/measuring/quantifying one or more samples, provide the comparison/score from one or more samples, obtain the comparison/score from one or more samples, administer a therapy, commence the administration of a therapy, cease the administration of a therapy, continue the administration of a therapy, temporarily interrupt the administration of a therapy, increase the amount of an administered therapeutic agent, decrease the amount of an administered therapeutic agent, continue the administration of an amount of a therapeutic agent, increase the frequency of administration of a therapeutic agent, decrease the frequency of administration of a therapeutic agent, maintain the same dosing frequency on a therapeutic agent, replace a therapy or therapeutic agent by at least another therapy or therapeutic agent, combine a therapy or therapeutic agent with at least another therapy or additional therapeutic agent.

In some aspects, a healthcare benefits provider can authorize or deny, for example, collection of a sample, processing of a sample, submission of a sample, receipt of a sample, transfer of a sample, analysis or measurement a sample, quantification of a sample, provision of results obtained after analyzing/measuring/quantifying a sample, transfer of results obtained after analyzing/measuring/quantifying a sample, comparison/scoring of results obtained after analyzing/measuring/quantifying one or more samples, transfer of the comparison/score from one or more samples, administration of a therapy or therapeutic agent, commencement of the administration of a therapy or therapeutic agent, cessation of the administration of a therapy or therapeutic agent, continuation of the administration of a therapy or therapeutic agent, temporary interruption of the administration of a therapy or therapeutic agent, increase of the amount of administered therapeutic agent, decrease of the amount of administered therapeutic agent, continuation of the administration of an amount of a therapeutic agent, increase in the frequency of administration of a therapeutic agent, decrease in the frequency of administration of a therapeutic agent, maintain the same dosing frequency on a therapeutic agent, replace a therapy or therapeutic agent by at least another therapy or therapeutic agent, or combine a therapy or therapeutic agent with at least another therapy or additional therapeutic agent.

In addition, a healthcare benefits can provide, e.g., authorize or deny the prescription of a therapy, authorize or deny coverage for therapy, authorize or deny reimbursement for the cost of therapy, determine or deny eligibility for therapy, etc.

In some aspects, a clinical laboratory can, for example, collect or obtain a sample, process a sample, submit a sample, receive a sample, transfer a sample, analyze or measure a sample, quantify a sample, provide the results obtained after analyzing/measuring/quantifying a sample, receive the results obtained after analyzing/measuring/quantifying a sample, compare/score the results obtained after analyzing/measuring/quantifying one or more samples, provide the comparison/score from one or more samples, obtain the comparison/score from one or more samples, or other related activities.

The assignment of a patient to a specific breast cancer class or classes disclosed herein (e.g., resulting from the application of a machine-learning classifier disclosed herein) can be applied, in addition to the treatment of patients or to the selection of a patient for treatment, to other therapeutic or diagnostic methods. For example, to methods to devise new methods of treatment (e.g., by selecting patients as candidates for a certain therapy or for participation in a clinical trial), to methods to monitor the efficacy of therapeutic agents, or to methods to adjust a treatment (e.g., formulations, dosage regimens, or routes of administration).

The methods disclosed herein can also include additional steps such as prescribing, initiating, and/or altering prophylaxis and/or treatment, based at least in part on the determination of the presence or absence of breast cancer or a specific breast cancer stage in a subject through the application of machine-learning based classifier disclosed herein.

The present disclosure also provides a method of determining whether to treat with a specific breast cancer therapy disclosed herein or a combination thereof a patient having a particular breast cancer phenotype or breast cancer stage identified through the application of a machine-learning based classifier disclosed herein. Also provided are methods of selecting a patient diagnosed with breast cancer or a specific stage of breast cancer as a candidate for treatment with a specific breast cancer therapy disclosed herein or a combination thereof based on the presence and/or absence of a particular breast cancer class identified through the application of a machine-learning based classifier disclosed herein.

In one aspect, the methods disclosed herein include making a diagnosis, which can be a differential diagnosis, based at least in part on the classification of the breast cancer in a subject, wherein the breast cancer has been classified through the application of a machine-learning based disclosed herein. This diagnosis can be recorded in a patient medical record. For example, in various aspects, the classification of the breast cancer status (e.g., presence/absence, stage, or a combination thereof), the diagnosis of the patient as treatable with a specific breads cancer therapy disclosed herein or a combination thereof, or the selected treatment, can be recorded in a medical record. The medical record can be in paper form and/or can be maintained in a computer-readable medium. The medical record can be maintained by a laboratory, physician's office, a hospital, a healthcare maintenance organization, an insurance company, and/or a personal medical record website.

In some aspects, a diagnosis, based on the application of a machine-learning based classifier disclosed herein can be recorded on or in a medical alert article such as a card, a worn article, and/or a radiofrequency identification (RFID) tag. As used herein, the term “worn article” refers to any article that can be worn on a subject's body, including, but not limited to, a tag, bracelet, necklace, or armband.

In some aspects, the sample can be obtained by a healthcare professional treating or diagnosing the patient, for measurement of the miRNA biomarker levels in the sample according to the healthcare professional's instructions (e.g., using a particular assay as described herein). In some aspects, the clinical laboratory performing the assay can advise the healthcare provider as to whether the patient can benefit from treatment with a specific breast cancer therapy disclosed herein or a combination thereof based on whether the patient's cancer is classified as belonging to a particular breast cancer class. In some aspects, results of a breast cancer classification (i.e., presence/absence, staging, or a combination thereof) conducted by applying a machine-learning based classifier disclosed herein can be submitted to a healthcare benefits provider for determination of whether the patient's insurance will cover treatment with a specific breast cancer therapy disclosed herein or a combination thereof. In some aspects, the clinical laboratory performing the assay can advise the healthcare provider as to whether the patient can benefit from treatment with a specific breast cancer therapy disclosed herein or combination thereof based on the breast cancer's classification.

### III. Breast Cancer Therapies

The method for recommending a breast cancer therapy based on the classifiers of the present disclosure may comprise steps in addition to those explicitly mentioned above. For example, further steps may relate, e.g., to isolating miRNAs from a sample, to the additional determination of other markers, to the use of an automatic device in the determination steps, or to the diagnosis of breast cancer prior to applying the method. As used herein, the term “therapy” refers to all measures applied to a subject to ameliorate the diseases or disorders referred to herein or the symptoms accompanied therewith to a significant extent. Said therapy as used herein also includes measures leading to an entire restoration of the health with respect to the diseases or disorders referred to herein.

It is to be understood that therapy as used in accordance with the present disclosure may not be effective in all subjects to be treated. However, the term shall require that a statistically significant portion of subjects being afflicted with a disease or disorder referred to herein can be successfully treated. Whether a portion is statistically significant can be determined without further ado by the person skilled in the art using various well-known statistical evaluation tools discussed herein above. The term “breast cancer therapy”, as used herein, relates to applying to a subject afflicted with breast cancer, including metastasizing breast cancer, measures to remove cancer cells from the subject, to inhibit growth of cancer cells, to kill cancer cells, or to cause the body of a patient to inhibit the growth of or to kill cancer cells.

In some aspects, the breast cancer therapy is chemotherapy, anti-hormone therapy, targeted therapy, immunotherapy, or any combination thereof. It is, however, also envisaged that the cancer therapy is radiation therapy or surgery, alone or combination with other therapy regimens. It is understood by the skilled person that the selection of the breast cancer therapy depends on several factors, like age of the subject, tumor staging, and receptor status of tumor cells. It is, however, also understood by the person skilled in the art, that the selection of the breast cancer therapy can be assisted by the methods of the present disclosure: if, e.g. breast cancer is diagnosed by the method for diagnosing breast cancer, but no metastatic breast cancer (MBC) is diagnosed by the method for diagnosing MBC, surgical removal of tumor may be sufficient. If, e.g. breast cancer is diagnosed by the method for diagnosing breast cancer and MBC is diagnosed by the method for diagnosing MBC, therapy measures in addition to surgery, e.g. chemotherapy and/or targeted therapy, may be appropriate. Likewise, if, e.g. breast cancer is diagnosed by the method for diagnosing breast cancer, and an unfavorable CTC status is determined by the method for determining the CTC status, e.g. a further addition of immunotherapy to the therapy regimen may be required.

As used herein, the term “chemotherapy” relates to treatment of a subject with an antineoplastic drug. In some aspects, the chemotherapy is a treatment including alkylating agents (e.g. cyclophosphamide), platinum (e.g. carboplatin), anthracyclines (e.g. doxorubicin, epirubicin, idarubicin, or daunorubicin) and topoisomerase II inhibitors (e.g. etoposide, irinotecan, topotecan, camptothecin, or VP16), anaplastic lymphoma kinase (ALK)-inhibitors (e.g. Crizotinib or AP26130), aurora kinase inhibitors (e.g. N-[4-[4-(4-Methylpiperazin-1-yl)-6-[(5-methyl-1 H-pyrazol-3-yl) amino]pyrimidin-2-yl]sulfanylphenyl]cyclopropanecarboxamide (VX-680)), antiangiogenic agents (e.g. Bevacizumab), or Iodine131-1-(3-iodobenzyl)guanidine (therapeutic metaiodobenzylguanidine), histone deacetylase (HDAC) inhibitors, alone or any suitable combination thereof. It is to be understood that chemotherapy, in some aspects, relates to a complete cycle of treatment, i.e. a series of several (e.g. four, six, or eight) doses of antineoplastic drug or drugs applied to a subject separated by several days or weeks without such application.

The term “anti-hormone therapy” relates to breast cancer therapy by blocking hormone receptors, e.g. estrogen receptor or progesterone receptor, expressed on tumor cells, or by blocking the biosynthesis of estrogen. Blocking of hormone receptors can be achieved by administering compounds, e.g. tamoxifen, binding specifically and thereby blocking the activity of said hormone receptors. Blocking of estrogen biosynthesis is achieved by administration of aromatase inhibitors like, e.g. anastrozole or letrozole. It is known to the skilled artisan that anti-hormone therapy is only advisable in cases where tumor cells are expressing hormone receptors.

The term “targeted therapy”, as used herein, relates to application to a patient a chemical substance known to block growth of cancer cells by interfering with specific molecules known to be necessary for tumorigenesis or cancer or cancer cell growth. Examples known to the skilled artisan are small molecules like, e.g. PARP-inhibitors (e.g. Iniparib), or monoclonal antibodies like, e.g., Trastuzumab.

The term “immunotherapy” as used herein relates to the treatment of cancer by modulation of the immune response of a subject. Said modulation may be inducing, enhancing, or suppressing said immune response. The term “cell based immunotherapy” relates to a breast cancer therapy comprising application of immune cells, e.g. T-cells, for example, tumor-specific NK cells, to a subject. The terms “radiation therapy” or “radiotherapy” is known to the skilled artisan. The term relates to the use of ionizing radiation to treat or control cancer. The skilled person also knows the term “surgery”, relating to operative measures for treating breast cancer, e.g. excision of tumor tissue.

### IV. Diagnostic Methods

In some aspects, the miRNAs of the present disclosure are used for diagnosing breast cancer, i.e., for example, the amount of said miRNAs is determined and the value obtained is compared to a reference, or used to derive a score, or used to train a model using machine learning. Measuring the amount of a miRNA is accomplished by, e.g., quantitative real-time PCR (qRT-PCR), or mass spectrometry. In one aspect, the amount of miRNAs of the present disclosure is determined using a detection agent.

As used herein, the term “detection agent” relates to an agent specifically interacting with, and thus recognizing, a miRNA of the present disclosure. In some aspects, the detection agent is a polynucleotide or an oligonucleotide. In some aspects, the detection agent is labeled in a way allowing detection of said detection agent by appropriate measures. Labeling can be done by various techniques well known in the art and depending of the label to be used. In some aspects, labels to be used are fluorescent labels comprising, inter alia, fluorochromes such as fluorescein, rhodamine, or Texas Red. However, the label may also be an enzyme or an antibody.

It is envisaged that an enzyme to be used as a label will generate a detectable signal by reacting with a substrate. Suitable enzymes, substrates and techniques are well known in the art. An oligonucleotide to be used as label may specifically recognize a target molecule which can be detected directly (e.g., a target molecule which is itself fluorescent) or indirectly (e.g., a target molecule which generates a detectable signal, such as an enzyme). The labeled detection agents of the sample will be contacted to the sample to allow specific interaction of the labeled detection agent with the miRNAs in the sample. Washing may be required to remove nonspecifically bound detection agents which otherwise would yield false values. After this interaction step is complete, a researcher will place the detection device into a reader device or scanner. A device for detecting fluorescent labels, for example, consists of some lasers, for example, a special microscope, and a camera. The fluorescent labels will be excited by the laser, and the microscope and camera work together to create a digital image of the sample. These data may be then stored in a computer, and a special program will be used, e.g., to subtract out background data. The resulting data are, for example, normalized, and may be converted into a numeric and common unit format. The data will be analyzed to compare samples to references and to identify significant changes. It is to be understood that the labeled detection agent need not necessarily detect the specific miRNA molecule isolated from the sample; the detection agent may also detect the amplification product obtained from said miRNA molecule, e.g., by PCR, qPCR, or qRT-PCR. It is, however, also envisaged that the detection agent is used without a label. In some aspects, the detection agent is bound to a solid surface and the sample, comprising miRNAs from a sample which have been labeled, are contacted with said surface-bound detection agent.

### V. Kits and Articles of Manufacture

The present disclosure further relates to a kit for carrying out a method for diagnosing breast cancer, wherein said kit comprises instructions for carrying out said method, a detection agent for determining the amount of at least one miRNA selected from the panel of microRNA biomarkers comprising, consisting, or consisting essentially of miR-150-5p, miR-106b-3p, miR-106a-5p, miR-125a-5p, miR-17-5p, miR-574-3p, miR-339-5p, miR-339-3p, miR-335-5p, miR-16-5p and miR-21-5p, or a subset thereof, e.g., miR-16-5p, miR-17-5p, miR-106a-5p, and miR-339-3p; miR-106a-5p, miR-17-5p, miR-339-3p, miR-16-5p and miR-335-5p; miR-106a-5p, miR-17-5p, miR-339-3p, miR-16-5p, miR-150-5p, miR-125a-5p, miR-339-5p, and miR-335-5p; miR-150-5p, miR-106b-3p, miR-106a-5p, miR-125a-5p, miR-17-5p, miR-574-3p, miR-339-5p, miR-339-3p, miR-335-5p, miR-16-5p and miR-21-5p; miR-150-5p, miR-106b-3p, miR-106a-5p, miR-125a-5p, miR-17-5p, miR-574-3p, miR-339-5p, miR-335-5p, miR-16-5p and miR-21-5p; or, miR-106a-5p, miR-150-5p, miR-125a-5p, miR-17-5p, miR-339-5p, miR-335-5p and miR-16-5p in a sample of a subject suspected to be afflicted with breast cancer, and standards for a reference.

In some aspects, the expression levels of a panel of miRNAs consisting of miR-16-5p, miR-17-5p, miR-106a-5p, and miR-339-3p are measured. In some aspects, the expression levels of a panel of miRNAs consisting of miR-106a-5p, miR-17-5p, miR-339-3p, miR-16-5p and miR-335-5p are measured. In some aspects, the expression levels of a panel of miRNAs consisting of miR-106a-5p, miR-17-5p, miR-339-3p, miR-16-5p, miR-150-5p, miR-125a-5p, miR-339-5p, and miR-335-5p are measured. In some aspects, the expression levels of a panel of miRNAs consisting of miR-150-5p, miR-106b-3p, miR-106a-5p, miR-125a-5p, miR-17-5p, miR-574-3p, miR-339-5p, miR-339-3p, miR-335-5p, miR-16-5p and miR-21-5p are measured. In some aspects, the expression levels of a panel of miRNAs consisting of miR-150-5p, miR-106b-3p, miR-106a-5p, miR-125a-5p, miR-17-5p, miR-574-3p, miR-339-5p, miR-335-5p, miR-16-5p and miR-21-5p are measured. In some aspects, the expression levels of a panel of miRNAs consisting of miR-106a-5p, miR-150-5p, miR-125a-5p, miR-17-5p, miR-339-5p, miR-335-5p and miR-16-5p are measured.

The present disclosure also relates to a kit for carrying out a method for diagnosing breast cancer, wherein said kit comprises instructions for carrying out said method, a detection agent for determining the amount of at least one miRNA selected from the panel of miRNA biomarkers comprising, consisting, or consisting essentially of miR-150-5p, miR-106b-3p, miR-106a-5p, miR-125a-5p, miR-17-5p, miR-574-3p, miR-339-5p, miR-339-3p, miR-335-5p, miR-16-5p and miR-21-5p, or a subset thereof, e.g., miR-16-5p, miR-17-5p, miR-106a-5p, and miR-339-3p; miR-106a-5p, miR-17-5p, miR-339-3p, miR-16-5p and miR-335-5p; miR-106a-5p, miR-17-5p, miR-339-3p, miR-16-5p, miR-150-5p, miR-125a-5p, miR-339-5p, and miR-335-5p; miR-150-5p, miR-106b-3p, miR-106a-5p, miR-125a-5p, miR-17-5p, miR-574-3p, miR-339-5p, miR-339-3p, miR-335-5p, miR-16-5p and miR-21-5p; miR-150-5p, miR-106b-3p, miR-106a-5p, miR-125a-5p, miR-17-5p, miR-574-3p, miR-339-5p, miR-335-5p, miR-16-5p and miR-21-5p; or, miR-106a-5p, miR-150-5p, miR-125a-5p, miR-17-5p, miR-339-5p, miR-335-5p and miR-16-5p in a sample of a subject suspected to be afflicted with metastatic breast cancer, and standards for a reference.

The term “kit” as used herein refers to a collection of the aforementioned compounds, means or reagents of the present disclosure that may or may not be packaged together. The components of the kit may be composed by separate vials (i.e. as a kit of separate parts) or provided in a single vial. Moreover, it is to be understood that the kit of the present disclosure is to be used for practicing the methods referred to herein above. In some aspects, all components are provided in a ready-to-use manner for practicing the methods referred to above. In some aspects, the kit contains instructions for carrying out the said methods. The instructions can be provided by a user's manual in paper- or electronic form. For example, the manual may comprise instructions for interpreting the results obtained when carrying out the aforementioned methods using the kit of the present disclosure.

The present disclosure also provides a kit comprising a plurality of oligonucleotide probes capable of specifically detecting a miRNA disclosed herein or combination thereof. Also provided is an article of manufacture comprising a plurality of oligonucleotide probes capable of specifically detecting a miRNA disclosed herein or combination thereof, wherein the article of manufacture comprises, e.g., a microarray.

Such kits and articles of manufacture can comprise containers, each with one or more of the various reagents (e.g., in concentrated form) utilized in the method, including, for example, one or more oligonucleotides (e.g., oligonucleotide capable of hybridizing to a miRNA corresponding to a biomarker miRNA disclosed herein).

One or more oligonucleotides can be provided already attached to a solid support. One or more oligonucleotides can be provided already conjugated to a detectable label. The kit can also provide reagents, buffers, and/or instrumentation to support the practice of the methods provided herein.

In some aspects, a kit comprises one or more nucleic acid probes (e.g., oligonucleotides comprising naturally occurring and/or chemically modified nucleotide units) capable of hybridizing a subsequence of a biomarker miRNA disclosed herein, e.g., under high stringency conditions. In some aspects, one or more nucleic acid probes (e.g., oligonucleotides comprising naturally occurring and/or chemically modified nucleotide units) capable of hybridizing a subsequence of the gene sequence of a biomarker miRNA disclosed herein, e.g., under high stringency conditions are attached to a microarray, e.g., a microarray chip. In some aspects, the microarray is, e.g., an Affymetrix, Agilent, Applied Microarrays, Arrayjet, or Illumina microarray. In some aspects, the array is a DNA microarray. In some aspects, the microarray is an RNA microarray or an oligonucleotide microarray.

A kit provided according to this disclosure can also comprise brochures or instructions describing the methods disclosed herein or their practical application to classify a patient's cancer sample. Instructions included in the kits can be affixed to packaging material or can be included as a package insert. While the instructions are typically written or printed materials they are not limited to such. Any medium capable of storing such instructions and communicating them to an end user is contemplated. Such media include, but are not limited to, electronic storage media (e.g., magnetic discs, tapes, cartridges, chips), optical media (e.g., CD ROM), and the like. As used herein, the term “instructions” can include the address of an internet site that provides the instructions.

The present disclosure also provides a kit for the detection of breast cancer, which comprises:
(i) specific oligonucleotides for reverse transcription of the group of miRNA selected from the set comprised by miR-16-5p, miR-17-5p, miR-106a-5p, miR-339-3p;
(ii) oligonucleotides for quantitative PCR of the miRNA pool selected from the pool comprised of miR-16-5p, miR-17-5p, miR-106a-5p, miR-339-3p; and a universal oligonucleotide Rv;
(iii) specific oligonucleotides for a miRNA control, e.g., a cel39 control;
(iv) optionally, synthetic positive controls for quantitative PCR step; and,
(v) optionally, a procedures manual.

The present disclosure also provides a kit for detecting breast cancer, comprising:
(i) specific oligonucleotides for reverse transcription of the group of miRNA selected from the set comprised by miR-106a-5p, miR-17-5p, miR-339-3p, miR-16-5p and miR-335-5p;
(ii) oligonucleotides for quantitative PCR of the miRNA pool selected from the pool comprised of miR-106a-5p, miR-17-5p, miR-339-3p, miR-16-5p, and miR-335-5p; and a universal oligonucleotide Rv;
(iii) specific oligonucleotides for a miRNA control, e.g., a cel39 control;
(iv) optionally, synthetic positive controls for quantitative PCR step; and,
(v) optionally, a procedures manual.

The present disclosure also provides a kit for the detection of breast cancer, which comprises:
(i) specific oligonucleotides for reverse transcription of the miRNA group selected from the set comprised by miR-106a-5p, miR-17-5p, miR-339-3p, miR-16-5p, miR-150-5p, miR-125a-5p, miR-339-5p, and miR-335-5p;
(ii) oligonucleotides for quantitative PCR of miRNA pool selected from the pool comprised of miR-106a-5p, miR-17-5p, miR-339-3p, miR-16-5p, miR-150-5p, miR-125a-5p, miR-339-5p, and miR-335-5p; and a universal oligonucleotide Rv;
(iii) specific oligonucleotides for a miRNA control, e.g., a cel39 control;
(iv) optionally, synthetic positive controls for quantitative PCR step; and,
(v) optionally, a procedures manual.

The present disclosure also provides a kit for the detection of breast cancer, which comprises:
(i) specific oligonucleotides for reverse transcription of the group of miRNA selected from the set comprised by miR-150-5p, miR-106b-3p, miR-106a-5p, miR-125a-5p, miR-17-5p, miR-574-3p, miR-339-5p, miR-339-3p, miR-335-5p, miR-16-5p, and miR-21-5p;
(ii) oligonucleotides for quantitative PCR of miRNA pool selected from the pool comprised of miR-150-5p, miR-106b-3p, miR-106a-5p, miR-125a-5p, miR-17-5p, miR-574-3p, miR-339-5p, miR-339-3p, miR-335-5p, miR-16-5p and miR-21-5p and a universal oligonucleotide Rv;
(iii) specific oligonucleotides for a miRNA control, e.g., a cel39 control;
(iv) optionally, synthetic positive controls for quantitative PCR step; and,
(v) optionally, a procedures manual.

The present disclosure also provides a kit for the detection of breast cancer, which comprises:
(i) specific oligonucleotides for reverse transcription of the group of miRNA selected from the set comprised by miR-150-5p, miR-106b-3p, miR-106a-5p, miR-125a-5p, miR-17-5p, miR-574-3p, miR-339-5p, miR-335-5p, miR-16-5p. and miR-21-5p;
(ii) oligonucleotides for quantitative PCR of miRNA pool selected from the pool comprised of miR-150-5p, miR-106b-3p, miR-106a-5p, miR-125a-5p, miR-17-5p, miR-574-3p, miR-339-5p, miR-335-5p, miR-16-5p. and miR-21-5p and a universal oligonucleotide Rv;
(iii) specific oligonucleotides for a miRNA control, e.g., a cel39 control;
(iv) optionally, synthetic positive controls for quantitative PCR step; and,
(v) optionally, a procedures manual.

The present disclosure also provides a kit for the detection of breast cancer, which comprises:
(i) specific oligonucleotides for reverse transcription of the group of miRNA selected from the set comprised by miR-106a-5p, miR-150-5p, miR-125a-5p, miR-17-5p, miR-339-5p, miR-335-5p and miR-16-5p;
(ii) oligonucleotides for quantitative PCR of miRNA pool selected from the pool comprised of miR-106a-5p, miR-150-5p, miR-125a-5p, miR-17-5p, miR-339-5p, miR-335-5p and miR-16-5p and a universal oligonucleotide Rv;
(iii) specific oligonucleotides for a miRNA control, e.g., a cel39 control;
(iv) optionally, synthetic positive controls for quantitative PCR step; and,
(v) optionally, a procedures manual.

### VI. Detection Arrays

The sets of miRNA biomarkers herein, e.g., the panel of miRNA biomarkers comprising, consisting or consisting essentially of miR-150-5p, miR-106b-3p, miR-106a-5p, miR-125a-5p, miR-17-5p, miR-574-3p, miR-339-5p, miR-339-3p, miR-335-5p, miR-16-5p and miR-21-5p, or a subset thereof, e.g., miR-16-5p, miR-17-5p, miR-106a-5p, and miR-339-3p; miR-106a-5p, miR-17-5p, miR-339-3p, miR-16-5p and miR-335-5p; miR-106a-5p, miR-17-5p, miR-339-3p, miR-16-5p, miR-150-5p, miR-125a-5p, miR-339-5p, and miR-335-5p; miR-150-5p, miR-106b-3p, miR-106a-5p, miR-125a-5p, miR-17-5p, miR-574-3p, miR-339-5p, miR-339-3p, miR-335-5p, miR-16-5p and miR-21-5p; miR-150-5p, miR-106b-3p, miR-106a-5p, miR-125a-5p, miR-17-5p, miR-574-3p, miR-339-5p, miR-335-5p, miR-16-5p and miR-21-5p; or, miR-106a-5p, miR-150-5p, miR-125a-5p, miR-17-5p, miR-339-5p, miR-335-5p and miR-16-5p, can be detected and quantitated for example by using a microarray.

The microarray can be prepared from gene-specific oligonucleotide probes generated from known miRNA sequences. The array may contain two different oligonucleotide probes for each miRNA, one containing the active, mature sequence and the other being specific for the precursor of the miRNA. The array may also contain controls, such as one or more mouse sequences differing from human orthologs by only a few bases, which can serve as controls for hybridization stringency conditions. tRNAs or other RNAs (e.g., rRNAs, mRNAs) from both species may also be printed on the microchip, providing an internal, relatively stable, positive control for specific hybridization. One or more appropriate controls for non-specific hybridization may also be included on the microchip. For this purpose, sequences are selected based upon the absence of any homology with any known miRNAs.

The microarray may be fabricated using techniques known in the art. For example, probe oligonucleotides of an appropriate length, e.g., 40 nucleotides, are 5′-amine modified at position C6 and printed using commercially available microarray systems, e.g., the GeneMachine OMNIGRID™ 100 Microarrayer and Amersham CODELINK™ activated slides. Labeled cDNA oligomer corresponding to the target RNAs is prepared by reverse transcribing the target RNA with labeled primer. Following first strand synthesis, the RNA/DNA hybrids are denatured to degrade the RNA templates. The labeled target cDNAs thus prepared are then hybridized to the microarray chip under hybridizing conditions, e.g., 6×SSPE/30% formamide at 25° C. for 18 hours, followed by washing in 0.75×TNT (Tris HCl/NaCl/Tween 20) at 37° C. for 40 minutes. At positions on the array where the immobilized probe DNA recognizes a complementary target cDNA in the sample, hybridization occurs. The labeled target cDNA marks the exact position on the array where binding occurs, allowing automatic detection and quantification. The output consists of a list of hybridization events, indicating the relative abundance of specific cDNA sequences, and therefore the relative abundance of the corresponding complementary miRs, in the patient sample. According to one aspect, the labeled cDNA oligomer is a biotin-labeled cDNA, prepared from a biotin-labeled primer. The microarray is then processed by direct detection of the biotin-containing transcripts using, e.g., Streptavidin-Alexa647 conjugate, and scanned utilizing conventional scanning methods. Image intensities of each spot on the array are proportional to the abundance of the corresponding miR in the patient sample.

The use of the array has several advantages for miRNA expression detection. First, the global expression of several hundred genes can be identified in the same sample at one time point. Second, through careful design of the oligonucleotide probes, expression of both mature and precursor molecules can be identified. Third, in comparison with Northern blot analysis, the chip requires a small amount of RNA, and provides reproducible results using 2.5 μg of total RNA. The relatively limited number of miRNAs (a few hundred per species) allows the construction of a common microarray for several species, with distinct oligonucleotide probes for each. Such a tool would allow for analysis of trans-species expression for each known miR under various conditions.

In addition to use for quantitative expression level assays of specific miRNAs, a microchip containing miRNA-specific probe oligonucleotides corresponding to a substantial portion of the miRNome, for example, the entire miRNome, may be employed to carry out miR gene expression profiling, for analysis of miR expression patterns. Distinct miRNA signatures can be associated with established disease markers, or directly with a disease state. In the context of the present disclosure, a miRNA signature can be obtained from the group of miRNA biomarkers comprising, consisting, or consisting essentially of miR-150-5p, miR-106b-3p, miR-106a-5p, miR-125a-5p, miR-17-5p, miR-574-3p, miR-339-5p, miR-339-3p, miR-335-5p, miR-16-5p and miR-21-5p, or a subset thereof, e.g., miR-16-5p, miR-17-5p, miR-106a-5p, and miR-339-3p; miR-106a-5p, miR-17-5p, miR-339-3p, miR-16-5p and miR-335-5p; miR-106a-5p, miR-17-5p, miR-339-3p, miR-16-5p, miR-150-5p, miR-125a-5p, miR-339-5p, and miR-335-5p; miR-150-5p, miR-106b-3p, miR-106a-5p, miR-125a-5p, miR-17-5p, miR-574-3p, miR-339-5p, miR-339-3p, miR-335-5p, miR-16-5p and miR-21-5p; miR-150-5p, miR-106b-3p, miR-106a-5p, miR-125a-5p, miR-17-5p, miR-574-3p, miR-339-5p, miR-335-5p, miR-16-5p and miR-21-5p; or, miR-106a-5p, miR-150-5p, miR-125a-5p, miR-17-5p, miR-339-5p, miR-335-5p and miR-16-5p.

According to the expression profiling methods described herein, total RNA from a sample from a subject suspected of having breast cancer is quantitatively reverse transcribed to provide a set of labeled target oligodeoxynucleotides complementary to the RNA in the sample. The target oligodeoxynucleotides are then hybridized to a microarray comprising miRNA-specific probe oligonucleotides to provide a hybridization profile for the sample. The result is a hybridization profile for the sample representing the expression pattern of miRNA in the sample. The hybridization profile comprises the signal from the binding of the target oligodeoxynucleotides from the sample to the miRNA-specific probe oligonucleotides in the microarray. The profile may be recorded as the presence or absence of binding (signal vs. zero signal). In some aspects, the profile recorded includes the intensity of the signal from each hybridization. The profile is compared to the hybridization profile generated from a normal, i.e., noncancerous, control sample. The profile can also be used to calculate a score, or as input to a machine learning model disclosed herein, wherein the output signal is indicative of the presence of, or propensity to develop, breast cancer in the subject.

### VII. Devices

The present disclosure also relates to a device for diagnosing breast cancer comprising:
(a) an analyzing unit comprising a detection agent for determining the expression of at least one miRNA selected from the group consisting of miRNAs miR-150-5p, miR-106b-3p, miR-106a-5p, miR-125a-5p, miR-17-5p, miR-574-3p, miR-339-5p, miR-339-3p, miR-335-5p, miR-16-5p and miR-21-5p, or a subset thereof, e.g., miR-16-5p, miR-17-5p, miR-106a-5p, and miR-339-3p; miR-106a-5p, miR-17-5p, miR-339-3p, miR-16-5p and miR-335-5p; miR-106a-5p, miR-17-5p, miR-339-3p, miR-16-5p, miR-150-5p, miR-125a-5p, miR-339-5p, and miR-335-5p; miR-150-5p, miR-106b-3p, miR-106a-5p, miR-125a-5p, miR-17-5p, miR-574-3p, miR-339-5p, miR-339-3p, miR-335-5p, miR-16-5p and miR-21-5p) miR-150-5p, miR-106b-3p, miR-106a-5p, miR-125a-5p, miR-17-5p, miR-574-3p, miR-339-5p, miR-335-5p, miR-16-5p and miR-21-5p; or, miR-106a-5p, miR-150-5p, miR-125a-5p, miR-17-5p, miR-339-5p, miR-335-5p and miR-16-5p in a sample of a subject suspected to be afflicted with breast cancer; and
(b) an evaluation unit comprising a data processor having tangibly embedded an algorithm for carrying out
(i) a comparison of the expression levels determined by the analyzing unit with a reference,
(ii) the calculation of a score based on the expression levels determined by the analyzing unit and comparison of the score to a reference value ort threshold,
(iii) the application of the expression levels determined by the analyzed unit to a machine learning model,
wherein the evaluation unit is capable of generating an output file containing a diagnosis established based on (i), (ii) or (iii).

The term “device” as used herein relates to a system of means comprising at least the aforementioned means operatively linked to each other as to allow the diagnosis. How to link the means in an operating manner will depend on the type of means included into the device. For example, where means for automatically determining the amount of the miRNAs of the present disclosure are applied, the data obtained by said automatically operating means can be processed by, e.g., a computer program in order to establish a diagnosis. Said device may accordingly include an analyzing unit for the measurement of the amount of the miRNAs of the present disclosure in a sample and an evaluation unit for processing the resulting data for the diagnosis. In such a case, the means are operatively linked in that the user of the system brings together the result of the determination of the amount and the diagnostic value thereof due to the instructions and interpretations given in a manual. The means may appear as separate devices in such an aspect and are, e.g., packaged together as a kit. The person skilled in the art will realize how to link the means without further inventive skills. In some aspects, the devices are those that can be applied without the particular knowledge of a specialized clinician, e.g., test stripes or electronic devices which merely require loading with a sample. The results may be given as output of parametric diagnostic raw data, e.g, as absolute or relative amounts. It is to be understood that these data will need interpretation by the clinician. However, also envisaged are expert system devices wherein the output comprises processed diagnostic raw data the interpretation of which does not require a specialized clinician. Further exemplary devices comprise the analyzing units/devices (e.g., biosensors, arrays, solid supports coupled to ligands specifically recognizing the miRNAs of the present disclosure, Plasmon surface resonance devices, NMR spectro-meters, mass-spectrometers etc.) or evaluation units/devices referred to above in accordance with the methods of the disclosure.

### VIII. Companion Diagnostic System

The methods disclosed herein can be provided as a companion diagnostic, for example available via a web server, to inform the clinician or patient about potential treatment choices. The methods disclosed herein can comprise collecting or otherwise obtaining a biological sample and performing an analytical method (e.g., apply a classifier disclosed herein) to classify a sample from a patient, and based on the classification assignment provide a suitable treatment for administration to the patient.

At least some aspects of the methods described herein, due to the complexity of the calculations involved can be implemented with the use of a computer. In some aspects, the computer system comprises hardware elements that are electrically coupled via bus, including a processor, input device, output device, storage device, computer-readable storage media reader, communications system, processing acceleration (e.g., DSP or special-purpose processors), and memory. The computer-readable storage media reader can be further coupled to computer-readable storage media, the combination comprehensively representing remote, local, fixed and/or removable storage devices plus storage media, memory, etc. for temporarily and/or more permanently containing computer-readable information, which can include storage device, memory and/or any other such accessible system resource.

A single architecture might be utilized to implement one or more servers that can be further configured in accordance with currently desirable protocols, protocol variations, extensions, etc. However, it will be apparent to those skilled in the art that aspects may well be utilized in accordance with more specific application requirements. Customized hardware might also be utilized and/or particular elements might be implemented in hardware, software or both. Further, while connection to other computing devices such as network input/output devices (not shown) may be employed, it is to be understood that wired, wireless, modem, and/or other connection or connections to other computing devices might also be utilized.

In one aspect, the system further comprises one or more devices for providing input data to the one or more processors. The system further comprises a memory for storing a dataset of ranked data elements. In another aspect, the device for providing input data comprises a detector for detecting the characteristic of the data element, e.g., such as a fluorescent plate reader, mass spectrometer, or gene chip reader.

The system additionally may comprise a database management system. User requests or queries can be formatted in an appropriate language understood by the database management system that processes the query to extract the relevant information from the database of training sets. The system may be connectable to a network to which a network server and one or more clients are connected. The network may be a local area network (LAN) or a wide area network (WAN), as is known in the art. In some aspects, the server includes the hardware necessary for running computer program products (e.g., software) to access database data for processing user requests. The system can be in communication with an input device for providing data regarding data elements to the system (e.g., expression values). In one aspect, the input device can include a gene expression profiling system including, e.g., a mass spectrometer, gene chip or array reader, and the like.

Some aspects described herein can be implemented so as to include a computer program product. A computer program product may include a computer readable medium having computer readable program code embodied in the medium for causing an application program to execute on a computer with a database. As used herein, a “computer program product” refers to an organized set of instructions in the form of natural or programming language statements that are contained on a physical media of any nature (e.g., written, electronic, magnetic, optical or otherwise) and that may be used with a computer or other automated data processing system. Such programming language statements, when executed by a computer or data processing system, cause the computer or data processing system to act in accordance with the particular content of the statements.

Computer program products include without limitation: programs in source and object code and/or test or data libraries embedded in a computer readable medium. Furthermore, the computer program product that enables a computer system or data processing equipment device to act in pre-selected ways may be provided in a number of forms, including, but not limited to, original source code, assembly code, object code, machine language, encrypted or compressed versions of the foregoing and any and all equivalents. In one aspect, a computer program product is provided to implement the treatment, diagnostic, prognostic, or monitoring methods disclosed herein, for example, to determine whether to administer a certain therapy based on the classification of sample from a patient according to the classifiers disclosed herein.

The computer program product includes a computer readable medium embodying program code executable by a processor of a computing device or system, the program code comprising:
(a) code that retrieves data attributed to a biological sample from a subject, wherein the data comprises miRNA expression level data (or data otherwise derived from miRNA expression level values) corresponding to miRNA biomarkers genes in the biological sample (e.g., a panel a miRNA biomarkers of the present disclosure). These values can also be combined with values corresponding, for example, the patient's current therapeutic regimen or lack thereof, and,
(b) code that executes a classification method that indicates, e.g., whether to administer an therapeutic agent to a patient in need thereof based on the classification of the patient sample by a machine learning-based classifier disclosed herein.

While various aspects have been described as methods or apparatuses, it should be understood that aspects can be implemented through code coupled with a computer, e.g., code resident on a computer or accessible by the computer. For example, software and databases could be utilized to implement many of the methods discussed above. Thus, in addition to aspects accomplished by hardware, it is also noted that these aspects can be accomplished through the use of an article of manufacture consisting of a computer usable medium having a computer readable program code embodied therein, which causes the enablement of the functions disclosed in this description. Therefore, it is desired that aspects also be considered protected by this patent in their program code means as well.

Furthermore, some aspects can be code stored in a computer-readable memory of virtually any kind including, without limitation, RAM, ROM, magnetic media, optical media, or magneto-optical media. Even more generally, some aspects could be implemented in software, or in hardware, or any combination thereof including, but not limited to, software running on a general purpose processor, microcode, PLAs, or ASICs.

It is also envisioned that some aspects could be accomplished as computer signals embodied in a carrier wave, as well as signals (e.g., electrical and optical) propagated through a transmission medium. Thus, the various types of information discussed above could be formatted in a structure, such as a data structure, and transmitted as an electrical signal through a transmission medium or stored on a computer readable medium.

### IX. Additional Techniques and Tests

Factors known in the art for diagnosing and/or suggesting, selecting, designating, recommending or otherwise determining a course of treatment for a patient or class of patients suspected of having cancer can be employed, e.g., in combination with measurements of the target sequence expression, or with the methods disclosed herein. Accordingly, the methods disclosed herein can include additional techniques such as cytology, histology, ultrasound analysis, MRI results, CT scan results, and measurements of PSA levels.

Certified tests for classifying disease status and/or designating treatment modalities can also be used in diagnosing, predicting, and/or monitoring the status or outcome of a cancer in a subject. A certified test can comprise a means for characterizing the expression levels of one or more of the target sequences of interest, and a certification from a government regulatory agency endorsing use of the test for classifying the disease status of a biological sample.

In some aspects, the certified test can comprise reagents for amplification reactions used to detect and/or quantitate expression of the target sequences to be characterized in the test. An array of probe nucleic acids can be used, with or without prior target amplification, for use in measuring target sequence expression.

The test can be submitted to an agency having authority to certify the test for use in distinguishing disease status and/or outcome. Results of detection of expression levels of the target sequences used in the test and correlation with disease status and/or outcome can be submitted to the agency. A certification authorizing the diagnostic and/or prognostic use of the test can be obtained.

Also provided are portfolios of expression levels comprising a plurality of normalized expression levels of any of the miRNA biomarker sets disclosed herein. In some aspects, the miRNA biomarkers are selected from a panel of miRNA biomarkers comprising, consisting, or consisting essentially of miR-150-5p, miR-106b-3p, miR-106a-5p, miR-125a-5p, miR-17-5p, miR-574-3p, miR-339-5p, miR-339-3p, miR-335-5p, miR-16-5p and miR-21-5p, or a subset thereof, e.g., miR-16-5p, miR-17-5p, miR-106a-5p, and miR-339-3p; miR-106a-5p, miR-17-5p, miR-339-3p, miR-16-5p and miR-335-5p; miR-106a-5p, miR-17-5p, miR-339-3p, miR-16-5p, miR-150-5p, miR-125a-5p, miR-339-5p, and miR-335-5p; miR-150-5p, miR-106b-3p, miR-106a-5p, miR-125a-5p, miR-17-5p, miR-574-3p, miR-339-5p, miR-339-3p, miR-335-5p, miR-16-5p and miR-21-5p; miR-150-5p, miR-106b-3p, miR-106a-5p, miR-125a-5p, miR-17-5p, miR-574-3p, miR-339-5p, miR-335-5p, miR-16-5p and miR-21-5p; or, miR-106a-5p, miR-150-5p, miR-125a-5p, miR-17-5p, miR-339-5p, miR-335-5p and miR-16-5p.

Such portfolios can be provided by performing the methods described herein to obtain miRNA expression levels from an individual patient or from a group of patients. The miRNA expression levels can be normalized by any method known in the art; exemplary normalization methods that can be used in various aspects include Robust Multichip Average (RMA), probe logarithmic intensity error estimation (PLIER), nonlinear fit (NLFIT) quantile-based and nonlinear normalization, and combinations thereof. Background correction can also be performed on the miRNA expression data; exemplary techniques useful for background correction include mode of intensities, normalized using median polish probe modeling and sketch-normalization.

In some aspects, portfolios are established such that the combination of miRNA biomarkers in the portfolio exhibit improved sensitivity and specificity relative to known methods. In considering a group of miRNA biomarkers for inclusion in a portfolio, a small standard deviation in expression measurements correlates with greater specificity. Other measurements of variation such as correlation coefficients can also be used in this capacity.

The disclosure also encompasses the above methods where the miRNA expression level determines the status or outcome of breast cancer in the subject with at least about 45% specificity, at least about 50% specificity, at least about 55%, at least about 60% specificity, at least about 65% specificity, at least about 70% specificity, at least about 75% specificity, at least about 80% specificity, at least about 85% specificity, at least about 90% specificity, or at least about 95% specificity. The disclosure also encompasses the above methods where the miRNA expression level determines the status or outcome of breast cancer in the subject with about 45% specificity, about 50% specificity, about 55% specificity, about 60% specificity, about 65% specificity, about 70% specificity, about 75% specificity, about 80% specificity, about 85% specificity, about 90% specificity, or about 95% specificity. The disclosure also encompasses the above methods where the miRNA expression level determines the status or outcome of breast cancer in the subject with between about 45% and about 50% specificity, between about 50% and about 55% specificity, between about 55% and about 60% specificity, between about 60% and about 65% specificity, between about 65% and about 70% specificity, between about 70% and about 75% specificity, between about 75% and about 80% specificity, between about 80% and about 85% specificity, between about 85% and about 90% specificity, between about 90% and about 95% specificity, between about 95% and about 100% specificity, between about 50% and about 60% specificity, between about 60% and about 70% specificity, between about 70% and about 80% specificity, between about 80% and about 90% specificity, between about 90% and about 100% specificity, between about 50% and about 65% specificity, between about 65% and about 80% specificity, between about 80% and about 95% specificity, between about 50% and about 70% specificity, between about 70% and about 90% specificity, between about 50% and about 75% specificity, or between about 75% and about 100% specificity.

The disclosure also encompasses the any of the methods disclosed herein where the accuracy of diagnosing, monitoring, and/or predicting a status or outcome of a breast cancer is at least about 45%, at least about 50%, at least about 55%, at least about 60%, at least about 65%, at least about 70%, at least about 75%, at least about 80%, at least about 85%, at least about 90%, or at least about 95%. The disclosure also encompasses the any of the methods disclosed herein where the accuracy of diagnosing, monitoring, and/or predicting a status or outcome of a breast cancer is about 45%, about 50%, about 55%, about 60%, about 65%, about 70%, about 75%, about 80%, about 85%, about 90%, or about 95%. The disclosure also encompasses the any of the methods disclosed herein where the accuracy of diagnosing, monitoring, and/or predicting a status or outcome of a breast cancer is between about 45% and about 50%, between about 50% and about 55%, between about 55% and about 60%, between about 60% and about 65%, between about 65% and about 70%, between about 70% and about 75%, between about 75% and about 80%, between about 80% and about 85%, between about 85% and about 90%, between about 90% and about 95%, between about 95% and about 100%, between about 50% and about 60%, between about 60% and about 70%, between about 70% and about 80%, between about 80% and about 90%, between about 90% and about 100%, between about 50% and about 65%, between about 65% and about 80%, between about 80% and about 95%, between about 50% and about 70% specificity, between about 70% and about 90%, between about 50% and about 75%, or between about 75% and about 100%.

The accuracy of a classifier or biomarker set may be determined by the 95% confidence interval (CI). Generally, a classifier or biomarker set is considered to have good accuracy if the 95% CI does not overlap 1. In some instances, the 95% CI of a classifier or biomarker is at least about 1.08, 1.10, 1.12, 1.14, 1.15, 1.16, 1.17, 1.18, 1.19, 1.20, 1.21, 1.22, 1.23, 1.24, 1.25, 1.26, 1.27, 1.28, 1.29, 1.30, 1.31, 1.32, 1.33, 1.34, or 1.35 or more. The 95% CI of a classifier or biomarker set may be at least about 1.14, 1.15, 1.16, 1.20, 1.21, 1.26, or 1.28. The 95% CI of a classifier or biomarker set may be less than about 1.75, 1.74, 1.73, 1.72, 1.71, 1.70, 1.69, 1.68, 1.67, 1.66, 1.65, 1.64, 1.63, 1.62, 1.61, 1.60, 1.59, 1.58, 1.57, 1.56, 1.55, 1.54, 1.53, 1.52, 1.51, 1.50 or less. The 95% CI of a classifier or biomarker set may be less than about 1.61, 1.60, 1.59, 1.58, 1.56, 1.55, or 1.53. The 95% CI of a classifier or biomarker set may be between about 1.10 to 1.70, between about 1.12 to about 1.68, between about 1.14 to about 1.62, between about 1.15 to about 1.61, between about 1.15 to about 1.59, between about 1.16 to about 1.160, between about 1.19 to about 1.55, between about 1.20 to about 1.54, between about 1.21 to about 1.53, between about 1.26 to about 1.63, between about 1.27 to about 1.61, or between about 1.28 to about 1.60.

In some instances, the accuracy of a biomarker set or classifier set is dependent on the difference in range of the 95% CI (e.g., difference in the high value and low value of the 95% CI interval). Generally, biomarker sets or classifiers with large differences in the range of the 95% CI interval have greater variability and are considered less accurate than biomarker sets or classifiers with small differences in the range of the 95% CI intervals. In some instances, a biomarker set or classifier is considered more accurate if the difference in the range of the 95% CI is less than about 0.60, 0.55, 0.50, 0.49, 0.48, 0.47, 0.46, 0.45, 0.44, 0.43, 0.42, 0.41, 0.40, 0.39, 0.38, 0.37, 0.36, 0.35, 0.34, 0.33, 0.32, 0.31, 0.30, 0.29, 0.28, 0.27, 0.26, 0.25 or less. The difference in the range of the 95% CI of a biomarker set or classifier may be less than about 0.48, 0.45, 0.44, 0.42, 0.40, 0.37, 0.35, 0.33, or 0.32. In some instances, the difference in the range of the 95% CI for a biomarker set or classifier is between about 0.25 to about 0.50, between about 0.27 to about 0.47, or between about 0.30 to about 0.45.

The disclosure also encompasses the any of the methods disclosed herein where the sensitivity is at least about 45%. In some aspects, the sensitivity is at least about 50%. In some aspects, the sensitivity is at least about 55%. In some aspects, the sensitivity is at least about 60%. In some aspects, the sensitivity is at least about 65%. In some aspects, the sensitivity is at least about 70%. In some aspects, the sensitivity is at least about 75%. In some aspects, the sensitivity is at least about 80%. In some aspects, the sensitivity is at least about 85%. In some aspects, the sensitivity is at least about 90%. In some aspects, the sensitivity is at least about 95%.

In some instances, the classifiers or biomarker sets disclosed herein are clinically significant. In some instances, the clinical significance of the classifiers or biomarker sets is determined by the AUC value. In order to be clinically significant, the AUC value is at least about 0.5, 0.55, 0.6, 0.65, 0.7, 0.75, 0.8, 0.85, 0.9, or 0.95. The clinical significance of the classifiers or biomarker sets can be determined by the percent accuracy. For example, a classifier or biomarker set is determined to be clinically significant if the accuracy of the classifier or biomarker set is at least about 50%, 55%, 60%, 65%, 70%, 72%, 75%, 77%, 80%, 82%, 84%, 86%, 88%, 90%, 92%, 94%, 96%, or 98%.

In other instances, the clinical significance of the classifiers or biomarker sets disclosed herein is determined by the median fold difference (MDF) value. In order to be clinically significant, the MDF value is at least about 0.8, 0.9, 1.0, 1.1, 1.2, 1.3, 1.4, 1.5, 1.6, 1.7, 1.9, or 2.0. In some instances, the MDF value is greater than or equal to 1.1. In other instances, the MDF value is greater than or equal to 1.2. Alternatively, or additionally, the clinical significance of the classifiers or biomarker sets is determined by the t-test P-value. In some instances, in order to be clinically significant, the t-test P-value is less than about 0.070, 0.065, 0.060, 0.055, 0.050, 0.045, 0.040, 0.035, 0.030, 0.025, 0.020, 0.015, 0.010, 0.005, 0.004, or 0.003. The t-test P-value can be less than about 0.050. Alternatively, the t-test P-value is less than about 0.010.

In some instances, the clinical significance of the classifiers or biomarker sets disclosed herein is determined by the clinical outcome. For example, different clinical outcomes can have different minimum or maximum thresholds for AUC values, MDF values, t-test P-values, and accuracy values that would determine whether the classifier or biomarker set is clinically significant. In another example, a classifier or biomarker set is considered clinically significant if the P-value of the t-test was less than about 0.08, 0.07, 0.06, 0.05, 0.04, 0.03, 0.02, 0.01, 0.005, 0.004, 0.003, 0.002, or 0.001. In some instances, the P-value may be based on any of the following comparisons: BCR vs non-BCR, CP vs non-CP, PCSM vs non-PCSM. For example, a classifier or biomarker set is determined to be clinically significant if the P-values of the differences between the KM curves for BCR vs non-BCR, CP vs non-CP, PCSM vs non-PCSM is lower than about 0.08, 0.07, 0.06, 0.05, 0.04, 0.03, 0.02, 0.01, 0.005, 0.004, 0.003, 0.002, or 0.001.

In some instances, the performance of a classifier or biomarker set of the preset disclosure is based on the odds ratio. A classifier or biomarker set may be considered to have good performance if the odds ratio is at least about 1.30, 1.31, 1.32, 1.33, 1.34, 1.35, 1.36, 1.37, 1.38, 1.39, 1.40, 1.41, 1.42, 1.43, 1.44, 1.45, 1.46, 1.47, 1.48, 1.49, 1.50, 1.52, 1.55, 1.57, 1.60, 1.62, 1.65, 1.67, 1.70 or more. In some instances, the odds ratio of a classifier or biomarker set is at least about 1.33.

The clinical significance of the classifiers and/or biomarker sets may be based on Univariable Analysis Odds Ratio P-value (uvaORPval). The Univariable Analysis Odds Ratio P-value (uvaORPval) of the classifier and/or biomarker set may be between about 0-0.4. The Univariable Analysis Odds Ratio P-value (uvaORPval) of the classifier and/or biomarker set may be between about 0-0.3. The Univariable Analysis Odds Ratio P-value (uvaORPval)) of the classifier and/or biomarker set may be between about 0-0.2. The Univariable Analysis Odds Ratio P-value (uvaORPval)) of the classifier and/or biomarker set may be less than or equal to 0.25, 0.22, 0.21, 0.20, 0.19, 0.18, 0.17, 0.16, 0.15, 0.14, 0.13, 0.12, 0.11. The Univariable Analysis Odds Ratio P-value (uvaORPval) of the classifier and/or biomarker set may be less than or equal to 0.10, 0.09, 0.08, 0.07, 0.06, 0.05, 0.04, 0.03, 0.02, 0.01. The Univariable Analysis Odds Ratio P-value (uvaORPval) of the classifier and/or biomarker set may be less than or equal to 0.009, 0.008, 0.007, 0.006, 0.005, 0.004, 0.003, 0.002, 0.001.

The clinical significance of the classifiers and/or biomarker set may be based on multivariable analysis Odds Ratio P-value (mvaORPval). The multivariable analysis Odds Ratio P-value (mvaORPval)) of the classifier and/or biomarker set may be between about 0-1. The multivariable analysis Odds Ratio P-value (mvaORPval)) of the classifier and/or biomarker set may be between about 0-0.9. The multivariable analysis Odds Ratio P-value (mvaORPval)) of the classifier and/or biomarker set may be between about 0-0.8. The multivariable analysis Odds Ratio P-value (mvaORPval) of the classifier and/or biomarker set may be less than or equal to 0.90, 0.88, 0.86, 0.84, 0.82, 0.80. The multivariable analysis Odds Ratio P-value (mvaORPval)) of the classifier and/or biomarker set may be less than or equal to 0.78, 0.76, 0.74, 0.72, 0.70, 0.68, 0.66, 0.64, 0.62, 0.60, 0.58, 0.56, 0.54, 0.52, 0.50. The multivariable analysis Odds Ratio P-value (mvaORPval) of the classifier and/or biomarker set may be less than or equal to 0.48, 0.46, 0.44, 0.42, 0.40, 0.38, 0.36, 0.34, 0.32, 0.30, 0.28, 0.26, 0.25, 0.22, 0.21, 0.20, 0.19, 0.18, 0.17, 0.16, 0.15, 0.14, 0.13, 0.12, 0.11. The multivariable analysis Odds Ratio P-value (mvaORPval)) of the classifier and/or biomarker set may be less than or equal to 0.10, 0.09, 0.08, 0.07, 0.06, 0.05, 0.04, 0.03, 0.02, 0.01. The multivariable analysis Odds Ratio P-value (mvaORPval)) of the classifier and/or biomarker set may be less than or equal to 0.009, 0.008, 0.007, 0.006, 0.005, 0.004, 0.003, 0.002, 0.001.

The clinical significance of the classifier and/or biomarker set may be based on the Kaplan Meier P-value (KM P-value). The Kaplan Meier P-value (KM P-value) of the classifier and/or biomarker set may be between about 0-0.8. The Kaplan Meier P-value (KM P-value) of the classifier and/or biomarker set may be between about 0-0.7. The Kaplan Meier P-value (KM P-value) of the classifier and/or biomarker set may be less than or equal to 0.80, 0.78, 0.76, 0.74, 0.72, 0.70, 0.68, 0.66, 0.64, 0.62, 0.60, 0.58, 0.56, 0.54, 0.52, 0.50. The Kaplan Meier P-value (KM P-value) of the classifier and/or biomarker set may be less than or equal to 0.48, 0.46, 0.44, 0.42, 0.40, 0.38, 0.36, 0.34, 0.32, 0.30, 0.28, 0.26, 0.25, 0.22, 0.21, 0.20, 0.19, 0.18, 0.17, 0.16, 0.15, 0.14, 0.13, 0.12, 0.11. The Kaplan Meier P-value (KM P-value) of the classifier and/or biomarker set may be less than or equal to 0.10, 0.09, 0.08, 0.07, 0.06, 0.05, 0.04, 0.03, 0.02, 0.01. The Kaplan Meier P-value (KM P-value) of the classifier and/or biomarker set may be less than or equal to 0.009, 0.008, 0.007, 0.006, 0.005, 0.004, 0.003, 0.002, 0.001.

The clinical significance of the classifier and/or biomarker set may be based on the survival AUC value (survAUC). The survival AUC value (survAUC) of the classifier and/or biomarker set may be between about 0-1. The survival AUC value (survAUC) of the classifier and/or biomarker set may be between about 0-0.9. The survival AUC value (survAUC) of the classifier and/or biomarker set may be less than or equal to 1, 0.98, 0.96, 0.94, 0.92, 0.90, 0.88, 0.86, 0.84, 0.82, 0.80. The survival AUC value (survAUC) of the classifier and/or biomarker set may be less than or equal to 0.80, 0.78, 0.76, 0.74, 0.72, 0.70, 0.68, 0.66, 0.64, 0.62, 0.60, 0.58, 0.56, 0.54, 0.52, 0.50. The survival AUC value (survAUC) of the classifier and/or biomarker set may be less than or equal to 0.48, 0.46, 0.44, 0.42, 0.40, 0.38, 0.36, 0.34, 0.32, 0.30, 0.28, 0.26, 0.25, 0.22, 0.21, 0.20, 0.19, 0.18, 0.17, 0.16, 0.15, 0.14, 0.13, 0.12, 0.11. The survival AUC value (survAUC) of the classifier and/or biomarker set may be less than or equal to 0.10, 0.09, 0.08, 0.07, 0.06, 0.05, 0.04, 0.03, 0.02, 0.01. The survival AUC value (survAUC) of the classifier and/or biomarker set may be less than or equal to 0.009, 0.008, 0.007, 0.006, 0.005, 0.004, 0.003, 0.002, 0.001

The clinical significance of the classifier and/or biomarker set may be based on the Univariable Analysis Hazard Ratio P-value (uvaHRPval). The Univariable Analysis Hazard Ratio P-value (uvaHRPval) of the classifier and/or biomarker set may be between about 0-0.4. The Univariable Analysis Hazard Ratio P-value (uvaHRPval) of the classifier and/or biomarker set may be between about 0-0.3. The Univariable Analysis Hazard Ratio P-value (uvaHRPval) of the classifier and/or biomarker set may be less than or equal to 0.40, 0.38, 0.36, 0.34, 0.32. The Univariable Analysis Hazard Ratio P-value (uvaHRPval) of the classifier and/or biomarker set may be less than or equal to 0.30, 0.29, 0.28, 0.27, 0.26, 0.25, 0.24, 0.23, 0.22, 0.21, 0.20. The Univariable Analysis Hazard Ratio P-value (uvaHRPval) of the classifier and/or biomarker set may be less than or equal to 0.19, 0.18, 0.17, 0.16, 0.15, 0.14, 0.13, 0.12, 0.11. The Univariable Analysis Hazard Ratio P-value (uvaHRPval) of the classifier and/or biomarker set may be less than or equal to 0.10, 0.09, 0.08, 0.07, 0.06, 0.05, 0.04, 0.03, 0.02, 0.01. The Univariable Analysis Hazard Ratio P-value (uvaHRPval) of the classifier and/or biomarker set may be less than or equal to 0.009, 0.008, 0.007, 0.006, 0.005, 0.004, 0.003, 0.002, 0.001.

The clinical significance of the classifier and/or biomarker set may be based on the Multivariable Analysis Hazard Ratio P-value (mvaHRPval)mva HRPval. The Multivariable Analysis Hazard Ratio P-value (mvaHRPval)mva HRPval of the classifier and/or biomarker set may be between about 0-1. The Multivariable Analysis Hazard Ratio P-value (mvaHRPval)mva HRPval of the classifier and/or biomarker set may be between about 0-0.9. The Multivariable Analysis Hazard Ratio P-value (mvaHRPval)mva HRPval of the classifier and/or biomarker set may be less than or equal to 1, 0.98, 0.96, 0.94, 0.92, 0.90, 0.88, 0.86, 0.84, 0.82, 0.80. The Multivariable Analysis Hazard Ratio P-value (mvaHRPval) mva HRPval of the classifier and/or biomarker set may be less than or equal to 0.80, 0.78, 0.76, 0.74, 0.72, 0.70, 0.68, 0.66, 0.64, 0.62, 0.60, 0.58, 0.56, 0.54, 0.52, 0.50. The Multivariable Analysis Hazard Ratio P-value (mvaHRPval) mva HRPval of the classifier and/or biomarker set may be less than or equal to 0.48, 0.46, 0.44, 0.42, 0.40, 0.38, 0.36, 0.34, 0.32, 0.30, 0.28, 0.26, 0.25, 0.22, 0.21, 0.20, 0.19, 0.18, 0.17, 0.16, 0.15, 0.14, 0.13, 0.12, 0.11. The Multivariable Analysis Hazard Ratio P-value (mvaHRPval)mva HRPval of the classifier and/or biomarker set may be less than or equal to 0.10, 0.09, 0.08, 0.07, 0.06, 0.05, 0.04, 0.03, 0.02, 0.01. The Multivariable Analysis Hazard Ratio P-value (mvaHRPval)mva HRPval of the classifier and/or biomarker set may be less than or equal to 0.009, 0.008, 0.007, 0.006, 0.005, 0.004, 0.003, 0.002, 0.001.

The clinical significance of a classifier or biomarker set may be based on the Multivariable Analysis Hazard Ratio P-value (mvaHRPval). In some aspects, the Multivariable Analysis Hazard Ratio P-value (mvaHRPval) of the classifier and/or biomarker may be between about 0 to about 0.60. In some aspects, the Multivariable Analysis Hazard Ratio P-value (mvaHRPval) of the classifier or biomarker set may be between about 0 to about 0.50. In some aspects, the Multivariable Analysis Hazard Ratio P-value (mvaHRPval) of the classifier or biomarker set may be less than or equal to 0.50, 0.47, 0.45, 0.43, 0.40, 0.38, 0.35, 0.33, 0.30, 0.28, 0.25, 0.22, 0.20, 0.18, 0.16, 0.15, 0.14, 0.13, 0.12, 0.11, 0.10. In some aspects, the Multivariable Analysis Hazard Ratio P-value (mvaHRPval) of the classifier or biomarker set may be less than or equal to 0.10, 0.09, 0.08, 0.07, 0.06, 0.05, 0.04, 0.03, 0.02, 0.01. In some aspects, the Multivariable Analysis Hazard Ratio P-value (mvaHRPval) of the classifier or biomarker set may be less than or equal to 0.01, 0.009, 0.008, 0.007, 0.006, 0.005, 0.004, 0.003, 0.002, 0.001.

The classifiers and/or biomarkers disclosed herein may outperform current classifier or biomarker sets in providing clinically relevant analysis of a sample from a subject. In some instances, the classifier or biomarker set may more accurately predict a clinical outcome or status as compared to current classifier or biomarker set. For example, a classifier or biomarker set may more accurately predict metastatic disease. Alternatively, a classifier or biomarker set may more accurately predict no evidence of disease. In some instances, the classifier or biomarker may more accurately predict death from a disease. The performance of a classifier or biomarker set disclosed herein may be based on the AUC value, odds ratio, 95% CI, difference in range of the 95% CI, p-value or any combination thereof.

The performance of the classifier or biomarker sets disclosed herein may be determined by AUC values and an improvement in performance may be determined by the difference in the AUC value of the classifier or biomarker disclosed herein and the AUC value of current classifier or biomarker set. In some instances, a classifier or biomarker set disclosed herein outperforms current classifier or biomarker set when the AUC value of the classifier or biomarker set disclosed herein is greater than the AUC value of the current classifier or biomarker set by at least about 0.05, 0.06, 0.07, 0.08, 0.09, 0.10, 0.11, 0.12, 0.13, 0.14, 0.15, 0.16, 0.17, 0.18, 0.19, 0.20, 0.022, 0.25, 0.27, 0.30, 0.32, 0.35, 0.37, 0.40, 0.42, 0.45, 0.47, 0.50 or more. In some instances, the AUC value of the classifier or biomarker set disclosed herein is greater than the AUC value of the current classifier or biomarker set by at least about 0.10. In some instances, the AUC value of the classifier or biomarker set disclosed herein is greater than the AUC value of the current classifier or biomarker set by at least about 0.13. In some instances, the AUC value of the classifier or biomarker set disclosed herein is greater than the AUC value of the current classifier or biomarker set by at least about 0.18.

The performance of the classifiers and/or biomarker sets disclosed herein may be determined by the odds ratios and an improvement in performance may be determined by comparing the odds ratio of the classifier or biomarker set disclosed herein and the odds ratio of current classifiers or biomarker set. Comparison of the performance of two or more classifiers or biomarker sets can be generally be based on the comparison of the absolute value of (1-odds ratio) of a first classifier or biomarker set to the absolute value of (1-odds ratio) of a second classifier or biomarker set. Generally, the classifier or biomarker set with the greater absolute value of (1-odds ratio) can be considered to have better performance as compared to the classifier or biomarker set with a smaller absolute value of (1-odds ratio).

In some instances, the performance of a first classifier or biomarker set is based on the comparison of the odds ratio and the 95% confidence interval (CI). For example, a first classifier or biomarker set may have a greater absolute value of (1-odds ratio) than a second classifier or biomarker set, however, the 95% CI of the first classifier or biomarker set may overlap 1 (e.g., poor accuracy), whereas the 95% CI of the second classifier or biomarker set does not overlap 1. In this instance, the second classifier or biomarker set is considered to outperform the first classifier or biomarker set because the accuracy of the first classifier or biomarker set is less than the accuracy of the second classifier or biomarker set. In another example, a first classifier or biomarker set may outperform a second classifier or biomarker set based on a comparison of the odds ratio; however, the difference in the 95% CI of the first classifier or biomarker set is at least about 2 times greater than the 95% CI of the second classifier or biomarker set. In this instance, the second classifier or biomarker set is considered to outperform the first classifier or biomarker set.

In some instances, a classifier or biomarker set disclosed herein more accurate than a current classifier or biomarker set. The classifier or biomarker disclosed herein is more accurate than a current classifier or biomarker set if the range of 95% CI of the classifier or biomarker set disclosed herein does not span or overlap 1 and the range of the 95% CI of the current classifier or biomarker set spans or overlaps 1.

In some instances, a classifier or biomarker set disclosed herein more accurate than a current classifier or biomarker set. The classifier or biomarker set disclosed herein is more accurate than a current classifier or biomarker set when difference in range of the 95% CI of the classifier or biomarker set disclosed herein is about 0.70, 0.60, 0.50, 0.40, 0.30, 0.20, 0.15, 0.14, 0.13, 0.12, 0.10, 0.09, 0.08, 0.07, 0.06, 0.05, 0.04, 0.03, 0.02 times less than the difference in range of the 95% CI of the current classifier or clinical variable. The classifier or biomarker set disclosed herein is more accurate than a current classifier or biomarker set when difference in range of the 95% CI of the classifier or biomarker set disclosed herein between about 0.20 to about 0.04 times less than the difference in range of the 95% CI of the current classifier or biomarker set.

### EXAMPLES

### Example 1

Methods Used for the Identification of Candidate miRNAs

Samples from patients with breast cancer (BC) and healthy donors (HD) were analyzed in order to identify and validate miRNAs to be used as biomarkers in the detection of BC.

To this end, clinical protocols were established and evaluated by various health institutions. Patients with BC and HD were enrolled between January 2017 and July 2021. The teaching and research and/or Ethics Committees of the institutions reviewed and approved the collection of blood samples and the collection of clinical and pathological data from the patients. The study was conducted in accordance with the ethical principles for medical research outlined in the Declaration of Helsinki, and signed informed consents were obtained from all patients and HD.

Clinical protocols were established and approved with the Hospital Militar Central Dr. Cosme Argerich Central Military Hospital (CABA) (APPROVAL DATE: 7 Jun. 2017), Instituto Quirurgico del Callao (CABA) (APPROVAL DATE 5 Jun. 2019), Hospital Municipal “Prof. Dr. Bernardo Houssay” (GBA) (DATE OF APPROVAL 27 Jul. 2017), Marie Curie Oncology Hospital (CABA) (DATE OF APPROVAL 12 Jan. 2017) and Hospital Interzonal General de Agudos “Professor Dr. Luis Güemes” (GBA) (DATE OF APPROVAL 10 Jun. 2018) to enroll patients diagnosed with BC. The pre-established patient inclusion criteria for the study were: women over 18 years of age with a diagnosis of breast adenocarcinoma of any subtype and stage, documented with or without a pathology report, who had not undergone surgery and/or therapy (chemotherapy, immunotherapy or radiation), and without a history of previous oncological disease.

On the other hand, protocols for HD were established at the Hospital Militar Central Dr. Cosme Argerich (CABA) (DATE OF APPROVAL: 7 Jun. 2017) and at the Hospital Municipal “Prof. Dr. Bernardo Houssay” (GBA) (DATE OF APPROVAL 4 Jun. 2021). The inclusion criteria were women over 18 years of age who did not have a previous or current diagnosis of cancer.

After signing the informed consent (IC), the intervening physician filled out an annex with affiliation and clinical data of the patients and drew a blood sample. Blood samples were obtained by venipuncture (a minimum of 8 ml and up to a maximum of 15 ml), which was placed in RNase-free sterile tubes containing 1 mL of 0.5 M EDTA pH 8. The samples were transported with triple wrapping to IBYME. Once the samples arrived at the laboratory, they were coded. The coding of the samples was carried out by the person in charge of the investigation, who changed the name, surname and ID of the patient for a unique alpha-numeric code in a totally confidential manner. Plasma was then isolated by centrifugation of the blood at 2000 rpm for 10 minutes and storing the samples at −70° C. under lock and key.

Patients and HD were separated into different cohorts: an exploratory cohort, where 30 HD and 36 HD were included, and a validation cohort, where 100 BC and 73 HD were included. The main characteristics of the patients are detailed in TABLE 1:

TABLE 1
Characteristics of the cohorts used in the
search and validation of candidate miRNAs:

| Cohort | Exploratory | Validatory |
|---|---|---|
| N Patients BCa | 30 | 100 |
| Age (SD) | 53 (12) | 58 (13) |

Stage

| | | |
|---|---|---|
| 0 | 1 | 11 |
| I | 4 | 42 |
| II | 11 | 19 |
| III | 13 | 4 |
| IV | 1 | 1 |
| No data | — | 23 |

Molecular subtype

| | | |
|---|---|---|
| Luminal A | 5 | 58 |
| Luminal B | 8 | 5 |
| Her2 | 7 | 5 |
| Triple Negative | 4 | 3 |
| No data | 5 | 18 |

Histologic type

| | | |
|---|---|---|
| In Situ | 2 | 11 |
| Infiltrant | 26 | 78 |
| No data | 2 | 11 |
| Ductal | 10 | 29 |
| Other | 18 | 60 |
| No data | 2 | 11 |

Grade

| | | |
|---|---|---|
| G1 | 1 | 27 |
| G2 | 10 | 45 |
| G3 | 13 | 2 |
| No data | 6 | 26 |
| N Healthy donors | 36 | 73 |
| Age (SD) | 49 (11) | 38 (12) |

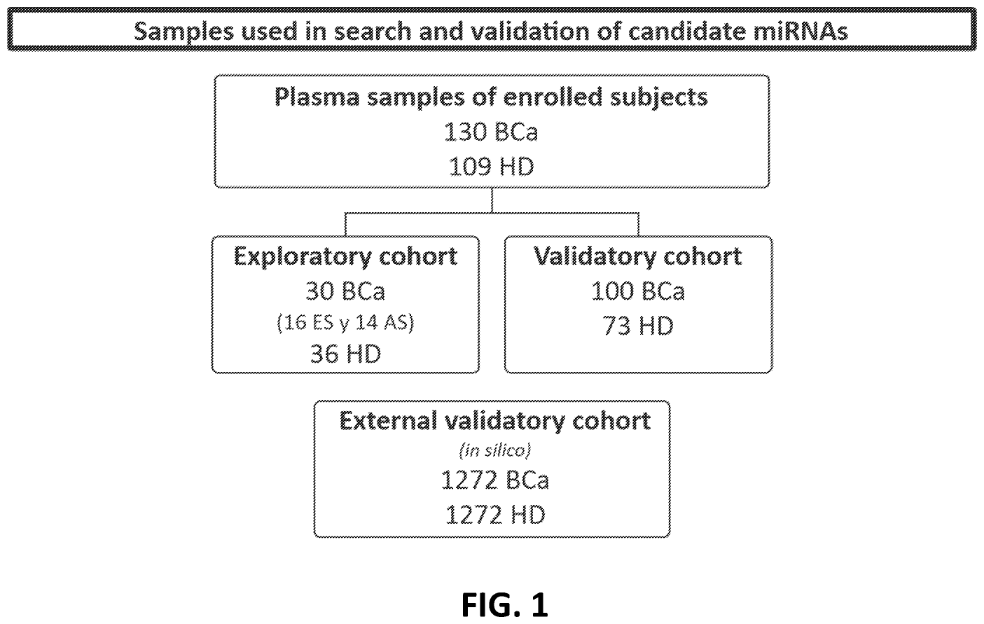

Plasma samples (n=239) from patients with BC and HD were used as detailed in FIG. 1.

The samples were divided into two cohorts: one exploratory and one validating. In the first, 30 patients with BC were included, which in turn were divided into early stages (ES) and advanced stages (AS), and were compared with 36 HD. Samples from the exploratory cohort were used for miRNA identification using expression microarrays and miRNA sequencing. In the validation cohort, 100 patients with BC and 73 HD were included, which were used for analytical validation (that is, by another technique: RT-qPCR) of the data obtained from the microarrays and the sequencing of miRNAs. Finally, an external validation cohort was used, which included miRNA expression microarray data from the serum of patients with BC and HD (1272 per group) obtained from public repositories.

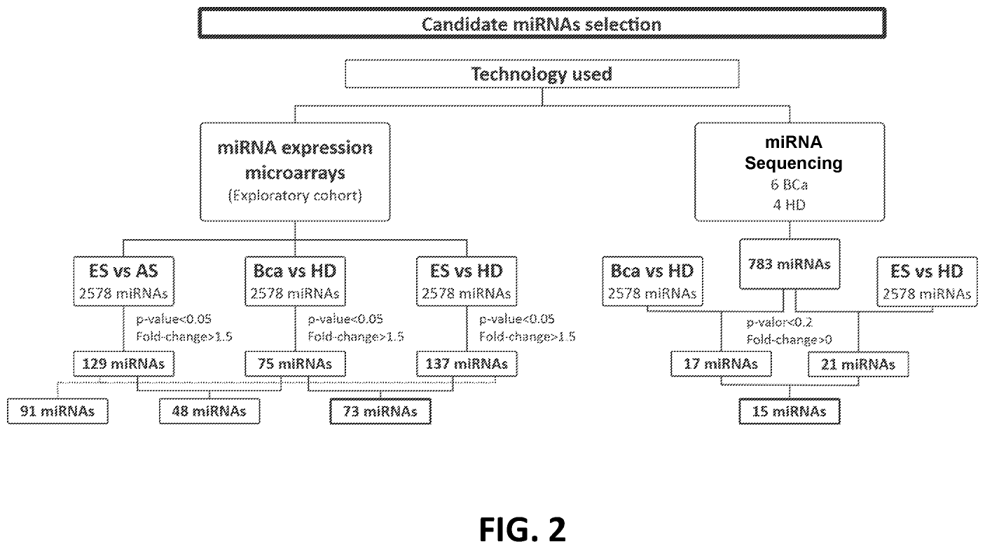

For the identification of candidate miRNAs for the early detection of BC, two technologies were used: expression microarrays (AFFYMETRIX®) and miRNA sequencing (GENOHUB®) as shown in FIG. 2.

For expression microarrays, plasma from 30 patients with BC and 36 HD, belonging to the exploratory cohort, was used. BC samples were divided into 5 subgroups according to stage and grouped as follows: Stage 0-IA (n=5), stage HA (n=6), stage IIB (n=5), stage IIIA (n=8), stage IIIB, IIIC and IV (n=6). miRNAs were then extracted from 800 μl of plasma from patients or HD in extractions of 1600 μl per column using the NUCLEOSPIN® miRNA Plasma kit (Macherey-Nagel) following the corresponding protocol. In the case of stages 0-IA, IIA, IIB and IIIB, IIIC and IV, 3 columns per group were used; in the case of stage IIIA, 4 columns were used, and finally, in the case of SV, they were separated into 4 groups, and a total of 20 columns were used. Two elutions were performed with 20 μl of molecular biology grade H₂O per column used, and all the elutions belonging to the corresponding group were pooled. They were then concentrated using the Jouan RCT 60 freeze-dryer (Thermofisher). Samples were resuspended in 11 μl H₂O RNAse-free. The concentration and purity of the miRNAs were evaluated using NanoDrop 2000, taking into account that 10% of the concentration calculated by NanoDrop corresponds to the amount of miRNAs as indicated in (Garcia-Elias et al. 2017).

Per microarray, a mass of 140 ng of circulating RNA was used to hybridize 9 miRNA expression microarrays GENECHIP® miRNA 4.0 Array (Affymetrix). Five were used for BC and 4 for HD. The hybridization was carried out in the technical service to third parties of the IFEVA (Faculty of Agronomy, UBA). Data normalization and analysis was performed using the EXPRESSION CONSOLE™ Software 1.3.1 and AFFYMETRIX® Transcriptome Analysis Console (TAC) programs. The differentially expressed miRNAs were identified by means of different analyzes detailed in FIG. 2. For the analyses, a p-value <0.05 and fold-change >1.5 were used as selection criteria. Thus, 3 groups of differentially expressed miRNAs were generated: a first group with 129 miRNAs increased in circulation from patients with BC in ES (0-IIB) compared to patients with BC in AS (IIIA-IV), a second group with 75 increased miRNAs in circulation of patients with BC compared to HD and a third group with 137 increased miRNAs in circulation of patients with BC in ES compared to HD.

Since the purpose was to identify those miRNAs that could be used for the early detection of the disease, new comparisons were made, where it was sought to identify those miRNAs that were in common between the aforementioned groups. In this way, 3 new groups were obtained as detailed in FIG. 2, one of 48 miRNAs, one of 91 miRNAs, and one of 73 miRNAs.

For miRNA sequencing, 1 mL of plasma from 6 patients with early-stage BC (n=2 Ia, n=2 Ib and n=2 Ic) and 4 HD were sent on dry ice to GenoHub (REAL SEQ BIOSCIENCES INC., US). There, the extraction of circulating miRNAs, the sequencing of the miRNAs and the corresponding statistical analysis were carried out. It was compared between BC and HD using the following systems and their versions: Ubuntu 18.04.2 LTS x86_64-pc-linux-gnu (64-bit), R version 3.6.0—“Planting of a Tree”, FastQC v0.11.5, cutadapt version 2.3, bowtie version 1.2.2, DESeq2_1.22.2, Rsubread_1.32.4, BiocParallel_1.16.6, Ggplot2_3.1.0.

The group of miRNAs corresponding to the sequencing was then compared with the candidate miRNAs obtained from the expression microarrays, as described in FIG. 2. From this analysis, a list of 783 miRNAs differentially expressed between BC and HD was obtained. Then, this list of miRNAs was compared with the miRNAs obtained from the expression microarrays, using a p-value <0.2 and a Fold-change >0 as selection criteria. The differentially expressed miRNAs identified by sequencing were compared, on the one hand, with the miRNAs identified in the expression microarrays when comparing BC vs HD and, on the other hand, with the miRNAs identified when comparing ES vs HD. From this new analysis, 2 groups of miRNAs in common were obtained, one of 17 and another of 21 respectively. Finally, these groups were compared and a final group of 15 miRNAs was obtained, with which the analysis was also continued.

Once the lists of candidate miRNAs were obtained, about 34 oligonucleotides were fine-tuned to be able to carry out the corresponding RT-qPCRs. For this, primers were designed following the Stem-loop method.

### Example 2

Validation of Candidate miRNAs

### Exploratory Cohort

Next, miRNAs were isolated from the plasma of patients with BC or HD in the exploratory cohort. One sample containing 1 μl from each BC patient (n=30) in this cohort and another containing 1 μl from each HD (n=36) were assembled. These combined samples were used for the set-up of the RT-qPCR of the 34 candidate miRNAs. Since the Stem-loop method used to identify miRNAs is usually complex, only 11 of the 34 primers were fine-tuned, with which the analysis was continued.

Once the RT-qPCR of the 11 candidate miRNAs was fine-tuned, their validation continued using stem-loop RT-qPCR. To do this, plasma RNA from 30 patients with BC and 36 HD belonging to the exploratory cohort was used, cDNA was synthesized and then qPCR was performed.

First, total RNA (including miRNAs) was isolated from patient plasma and HD was performed using Tri-Reagent (Molecular Research Center). To 200 μl of plasma, 10 fmol of a synthetic miRNA (spike in =cel-miR-39-3p) was added and then 600 μl of Trireagent was added. It was homogenized, incubated at room temperature for 5 minutes and then 120 μl of chloroform were added. It was shaken vigorously for a few seconds and then incubated at room temperature for 2-3 minutes. After incubation, it was centrifuged at 12,000 rpm for 15 minutes at 4° C. Then 350 μl of the aqueous phase were taken, 300 μl of isopropanol was added and the samples were incubated for approximately 16 hours at −20° C. The next day, it was centrifuged at 12,000 rpm for 10 minutes at 4° C., the supernatant was discarded, and 2 washes were performed with cold 75% EtOH, centrifuging after each wash at 7,500 rpm for 5 minutes at 4° C. After the washings, the pellet was left to dry at 55-60° C. for a few minutes until the EtOH had completely evaporated. The pellet was resuspended in 22 μl of RNase-free water and incubated for 10 minutes at 55-60° C. The samples were then stored at −70° C. The concentration and purity in this case were not evaluated, since, being very low concentrations, the NanoDrop does not manage to analyze the sample with certainty.

Reverse transcription was performed using the Stem-Loop method described in (Chen et al. (2005) Nucleic Acids Research 33 (20): e179). cDNA was synthesized from 4 μl of total RNA using the M-MLV Reverse Transcriptase kit (PROMEGA). RNA, RNase-free water and a mix of up to 6 specific stem-loop primers for each miRNA (1 μl of each specific STEM primer is used), which were used to elongate the original product, were mixed and incubated at 70° C. for 5 minutes. Then a mix of 4 μl of 5× buffer specific for the enzyme, 1 μl of 10 mM dNTPs and 1 μl of RT enzyme was added. It was then incubated for 30 minutes at 16° C., a key step for the stem-loop technique, then 60 minutes at 42° C. for synthesis and 5 minutes at 70° C. to inactivate the enzyme.

To perform the qPCR, 1 μl of pure cDNA, 3.5 μl of ROUX water, 0.5 μl of a mix of primers Fw specific for the miRNA to be detected and universal Rv that pairs with the stem-loop and 5 μl of the mix FastStart Universal SYBR Green Master (ROCHE). The PCR reaction was carried out in duplicate in a StepOne Plus equipment (Applied Biosystems) or in a CFX96 Touch Real-Time PCR Detection System (Bio-Rad). Amplification cycling included 1 cycle of 2 min at 50° C.; 1 cycle of 10 min at 95° C.; 40 cycles of: 15s at 95° C., 15s at 65° C. and 1 min at 60° C.; then the fluorescence reading and finally the melting curve from 60 to 95° C.+0.3° C. every 6 s.

Primers used were designed and purchased from the company Macrogen, which are listed in TABLE 2.

The design of the primers for the RT-qPCR technique was carried out by adapting the guidelines present in the work of Chen et al. (Chen et al. (2005) Nucleic Acids Research 33 (20): e179). For this, the mature miRNA sequence was downloaded using the miRBase database (http://www.mirbase.org/). Then, for the design of the stem-loop primer for the RT, a stem-loop type sequence (GTCTCCTCTGGTGCAGGGTCCGAGGTATTCGCACCAGAGGAGAC) (SEQ ID NO: 40) was used, followed by a sequence of six complementary bases and written 5′-3′ to the last six nucleotides at the end. 5′ of the mature miRNA. For the design of the Forward primer for qPCR, the sequence of the mature miRNA without the last 6 bases of the 5′ end was used. Then, to extend the length of the primer, a short C and G sequence was added to the 5′ end (for example: GCGGCGG; SEQ ID NO: 39). Finally, to create the Reverse primer that will be universal to all miRNAs, a complementary sequence to the stem-loop sequence was designed. Melting temperature (Tm), self-complementarity, and nonspecific products were analyzed using the Primer Blast tool (https://www.ncbi.nlm.nih.gov/tools/primer-blast/). The exclusion criteria were an optimal Tm of 60° C. with a range of +/−5° C., self-complementarity less than 4, and 3′ self-complementarity also less than 4.

TABLE 2
List of primers used for the Stem-loop RT-qPCR
technique from plasma

| | | | SEQ |
|---|---|---|---|
| | | T ann | ID |
| Primer | Sequence (5′-3′) | (° C.) | NO |
| RT-Stem-loop-Rv | TGGTGCAGGGTCCGAGGTATT | — | 13 |
| RT-hsa-miR-21-5p-STEM | GTCTCCTCTGGTGCAGGGTCCGAGGTATTCGC | — | 14 |
| | ACCAGAGGAGACTCAACA | | |
| RT-hsa-miR-21-5p Fw | CGGGGGGTAGCTTATCAGACTG | 65 | 15 |
| RT-hsa-miR-106a-5p- | GTCTCCTCTGGTGCAGGGTCCGAGGTATTCGC | — | 16 |
| STEM | ACCAGAGGAGACCTACCT | | |
| RT-hsa-miR-106a-5p Fw | GGCGGAAAAGTGCTTACAGTGC | 65 | 17 |
| RT-hsa-miR-125a-5p- | GTCTCCTCTGGTGCAGGGTCCGAGGTATTCGC | — | 18 |
| STEM | ACCAGAGGAGACTCACAG | | |
| RT-hsa-miR-125a-5p Fw | CCCCCTCCCTGAGACCCTTTAAC | 65 | 19 |
| RT-hsa-miR-17-5p-STEM | GTCTCCTCTGGTGCAGGGTCCGAGGTATTCGC | — | 20 |
| | ACCAGAGGAGACCTACCT | | |
| RT-hsa-miR-17-5p Fw | GGATGGCAAAGTGCTTACAGTGC | 65 | 21 |
| RT-hsa-miR-106b-3p- | GTCTCCTCTGGTGCAGGGTCCGAGGTATTCGC | — | 22 |
| STEM | ACCAGAGGAGACGCAGCA | | |
| RT-hsa-miR-106b-3p Fw | GGGCCGCACTGTGGGTAC | 65 | 23 |
| RT-hsa-miR-150-5p-STEM | GTCTCCTCTGGTGCAGGGTCCGAGGTATTCGC | — | 24 |
| | ACCAGAGGAGACCACTGG | | |
| RT-hsa-miR-150-5p Fw | CCCCCTCTCCCAACCCTTGT | 65 | 25 |
| RT-hsa-miR-16-5p-STEM | GTCTCCTCTGGTGCAGGGTCCGAGGTATTCGC | — | 26 |
| | ACCAGAGGAGACCGCCAA | | |
| RT-hsa-miR-16-5p Fw | GGCCCGTAGCAGCACGTAAATA | 65 | 27 |
| RT-hsa-miR-335-5p-STEM | GTCTCCTCTGGTGCAGGGTCCGAGGTATTCGC | — | 28 |
| | ACCAGAGGAGACACATTT | | |
| RT-hsa-miR-335-5p Fw | CGGCGGTCAAGAGCAATAACG | 65 | 29 |
| RT-hsa-miR-339-3p-STEM | GTCTCCTCTGGTGCAGGGTCCGAGGTATTCGC | — | 30 |
| | ACCAGAGGAGACCGGCTC | | |
| RT-hsa-miR-339-3p Fw | GGTGAGCGCCTCGACGAC | 65 | 31 |
| RT-hsa-miR-339-5p-STEM | GTCTCCTCTGGTGCAGGGTCCGAGGTATTCGC | — | 32 |
| | ACCAGAGGAGACCGTGAG | | |
| RT-hsa-miR-339-5p Fw | CCCTCCCTGTCCTCCAGGAG | 65 | 33 |
| RT-hsa-miR-574-3p-STEM | GTCTCCTCTGGTGCAGGGTCCGAGGTATTCGC | — | 34 |
| | ACCAGAGGAGACTGTGGG | | |
| RT-hsa-miR-574-3p Fw | CGCCACGCTCATGCACAC | 65 | 35 |
| RT-cel-miR-39-3p-STEM | GTCTCCTCTGGTGCAGGGTCCGAGGTATTCGC | — | 36 |
| | ACCAGAGGAGACCAAGCT | | |
| RT-cel-miR-39-3p Fw | CGGGGTCACCGGGTGTAAATC | 65 | 37 |

The calculation of the expression levels of the miRNAs analyzed was performed using the ΔΔCT method as mentioned above, normalizing the expression levels of the miRNA of interest to cel-miR-39-3p. In all cases, the NRT negative control was run (RNA pool from all samples from patients and volunteers) that had been incubated under the conditions of the RT reaction, but in the absence of the reverse transcriptase enzyme. Finally, the average value and standard deviation of this value obtained in “n” biological replicates were calculated.

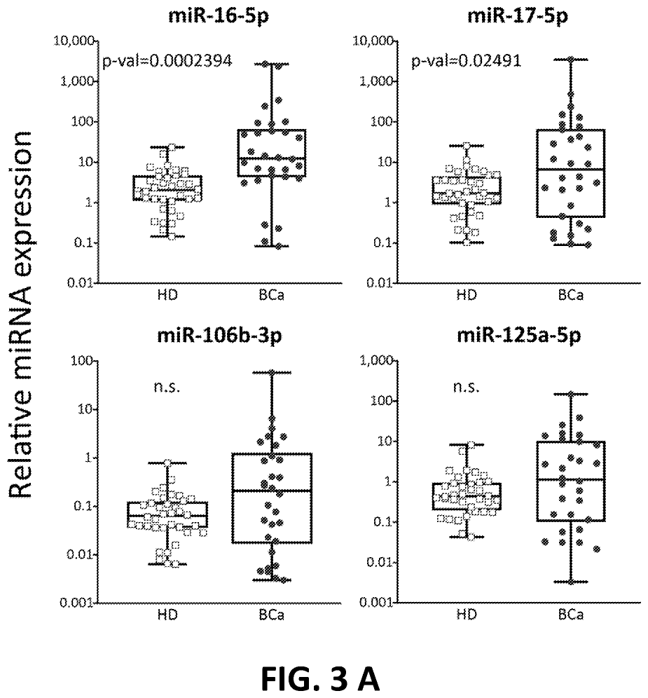

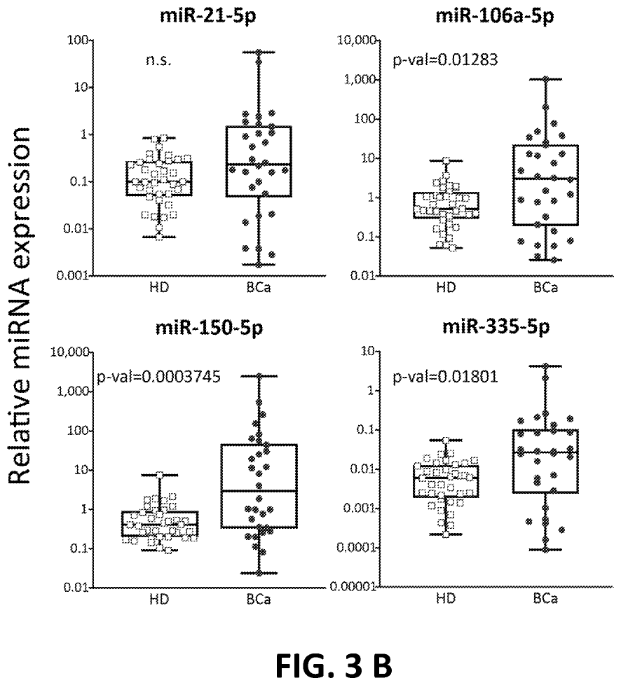

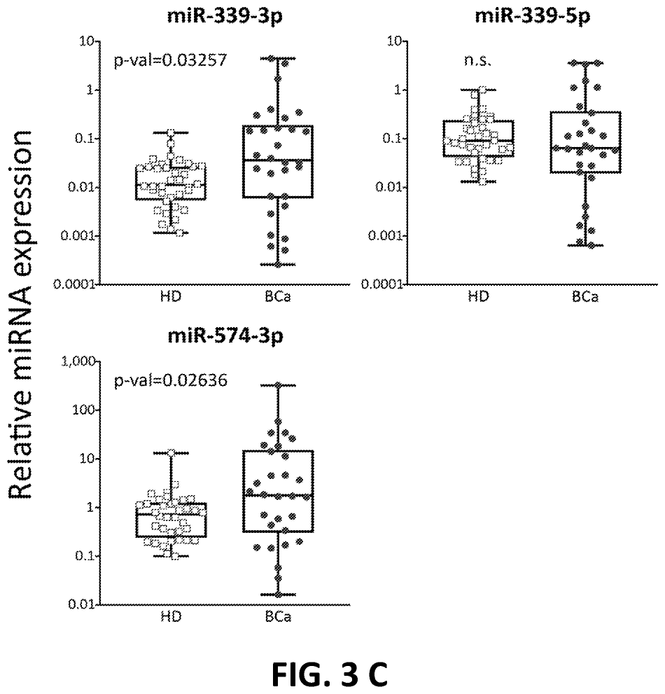

Of the 11 candidate miRNAs, the miRNAs miR-16-5p, miR-17-5p, miR-106a-5p, miR-150-5p, miR-335-5p, miR-339-3p and miR-574-3p were found increased in the circulation of patients with BC compared to HD, in line with what was found from the microarrays of expression and the sequencing of miRNAs, while no significant differences were found in the case of miRNAs miR-21-5p, miR-106b-3p, miR-125a-5p and miR-339-5p (FIGS. 3A, 3B, 3C).

Using the qPCR expression data, a ROC curve was performed for each of the candidate miRNAs. It provided information about how good a biomarker the miRNA was to be able to differentiate between a patient with BC from a HD. For this purpose, the value of the area under the curve or AUC was calculated. Its values ranged between 0.5 and 1. An AUC value of 0.5 indicated that miRNA expression did not serve to discriminate between sick and healthy subjects, since it has a 50% chance of detecting as sick and a 50% change of detecting as sick. For healthy subjects, an AUC between 0.7 and 0.8 was considered acceptable, between 0.8 and 0.9 was considered very good, and above 0.9 was considered excellent.

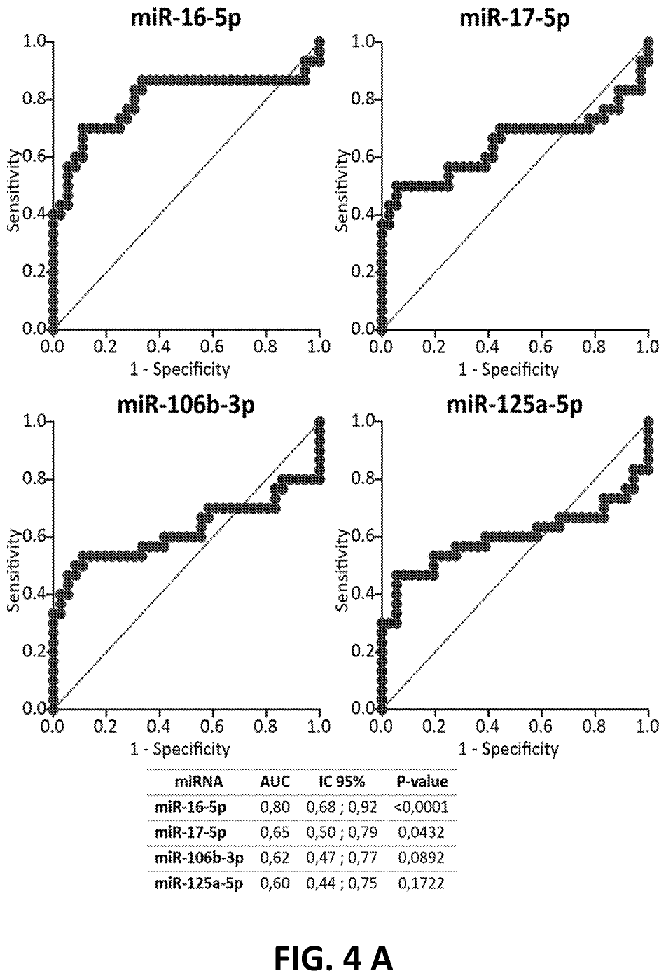

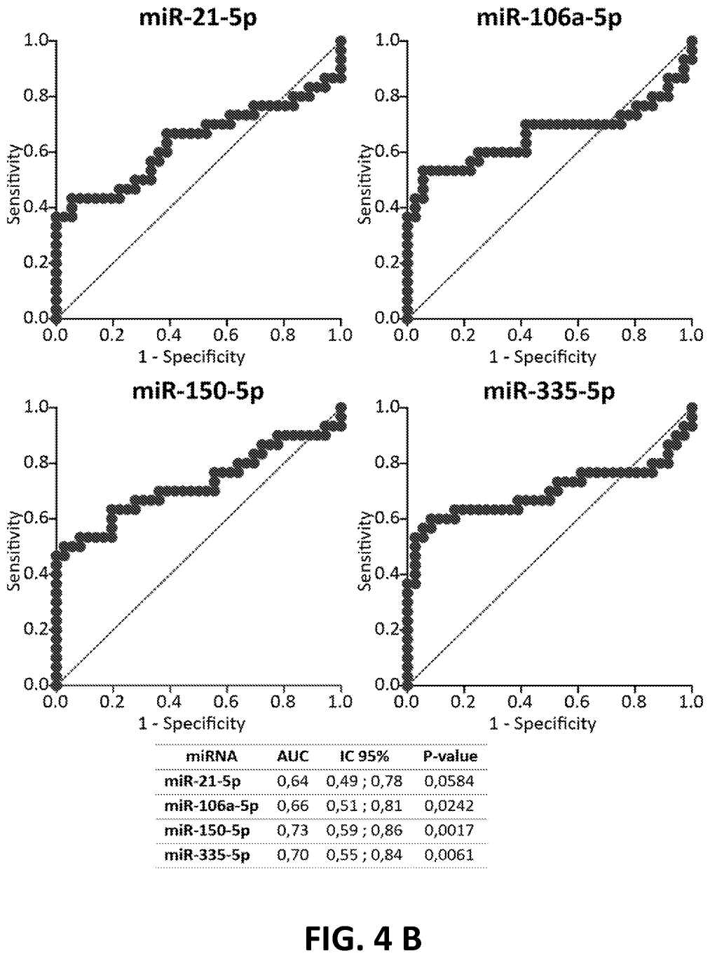

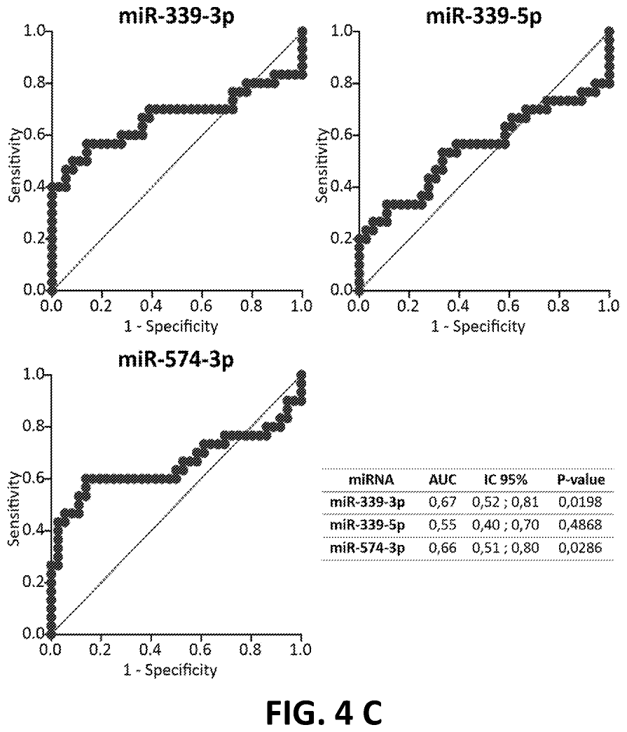

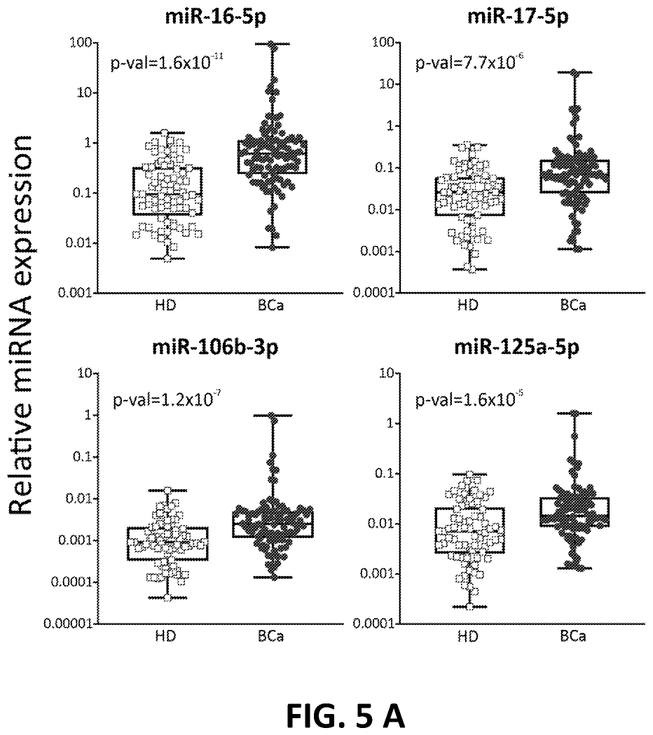

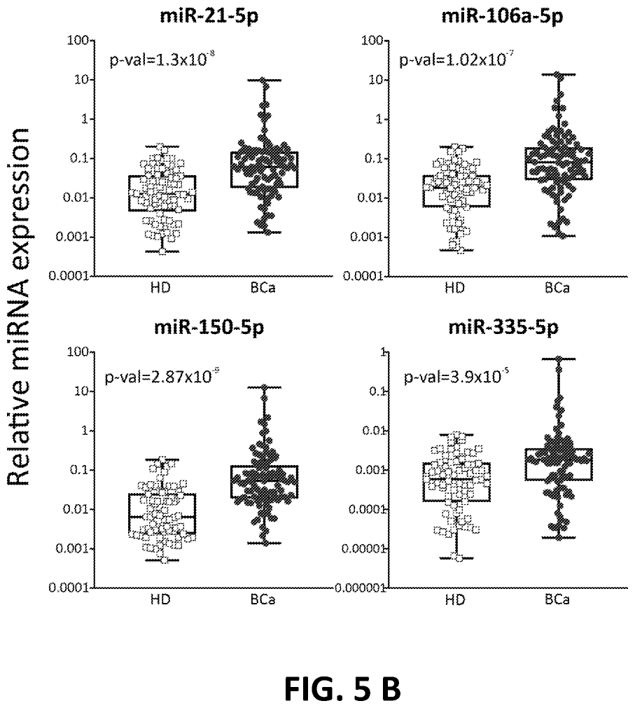

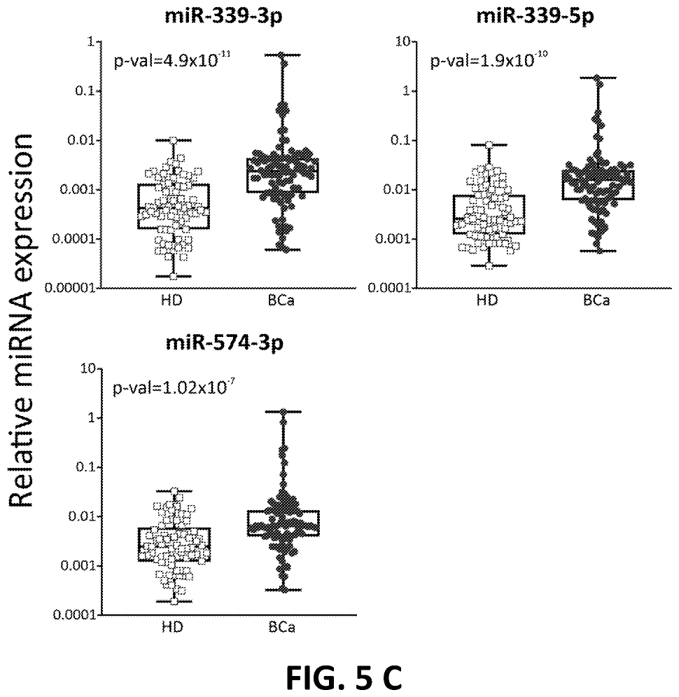

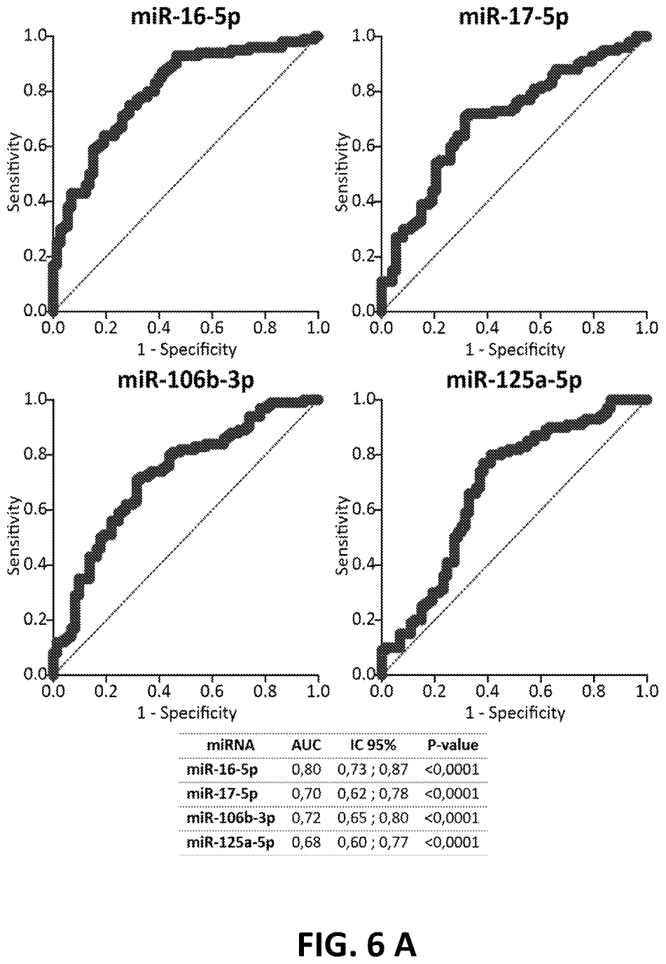

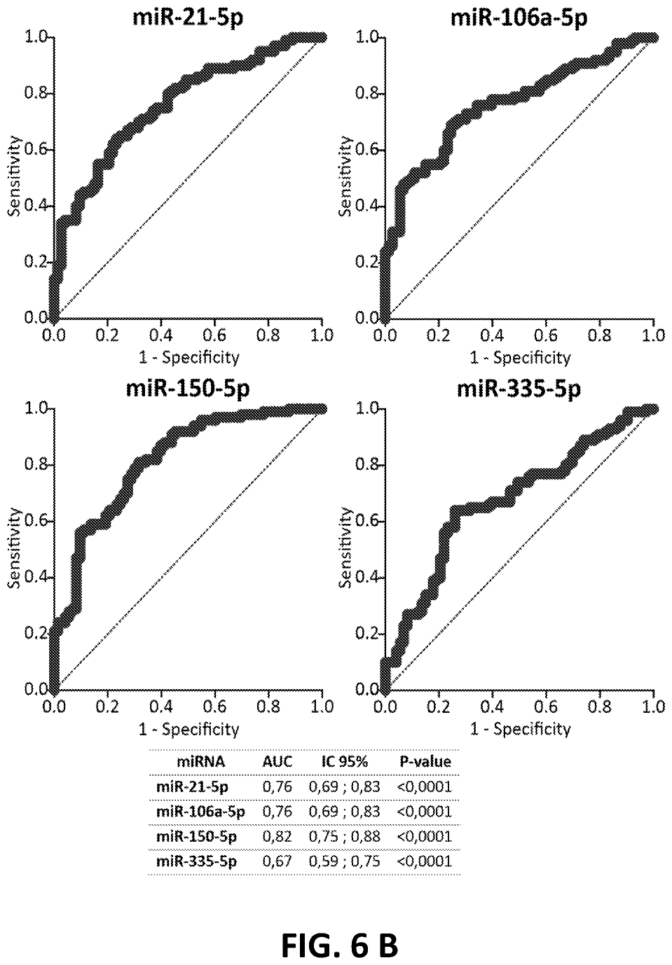

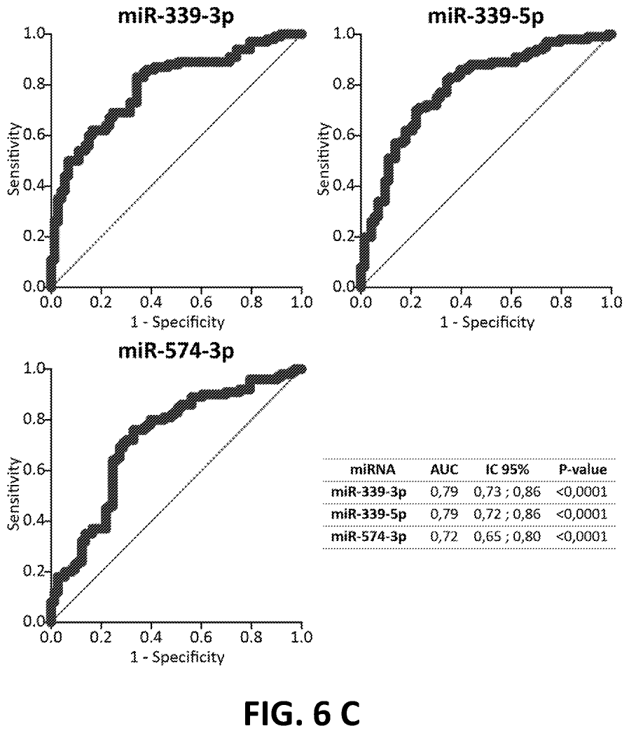

FIGS. 4A, 4B, 4C shows that the miRNAs miR-16-5p, miR-17-5p, miR-106a-5p, miR-150-5p, miR-335-5p, miR-339-3p and miR-574-3p are good biomarkers to differentiate between healthy and diseased subjects, given that their AUC values were above 0.5, and most of them were in fact between 0.6 and 0.8. On the other hand, the miRNAs miR-21-5p, miR-106b-3p, miR-125a-5p and miR-339-5p were not good at differentiating between healthy and diseased given that their AUCs were close to 0.5.

### Validation Cohort

A second independent cohort of patients with BC and HD with a larger number of samples was used to perform a more robust validation, since these new individuals (except for the 10 used for miRNA sequencing) had not been used in the identification stage of the biomarkers. Therefore, circulating miRNAs were isolated from plasma of 100 patients with BC and 73 with HD, cDNA was synthesized, and the respective qPCRs were performed for the 11 identified miRNAs.

In this case, it was observed that the 11 miRNAs were significantly increased in the circulation of patients with BC compared to HD (FIGS. 4A, 4B, 4C), in concordance with what was found from expression microarrays and miRNA sequencing.

In turn, all the ROC curves associated with each of the miRNAs in this second cohort of more individuals were also performed, and it was found that the 11 miRNAs were good biomarkers to differentiate a sick patient from a healthy individual, since the AUC values in this case were approximately between 0.7 and 0.8.

### In Silico External Validation Cohort

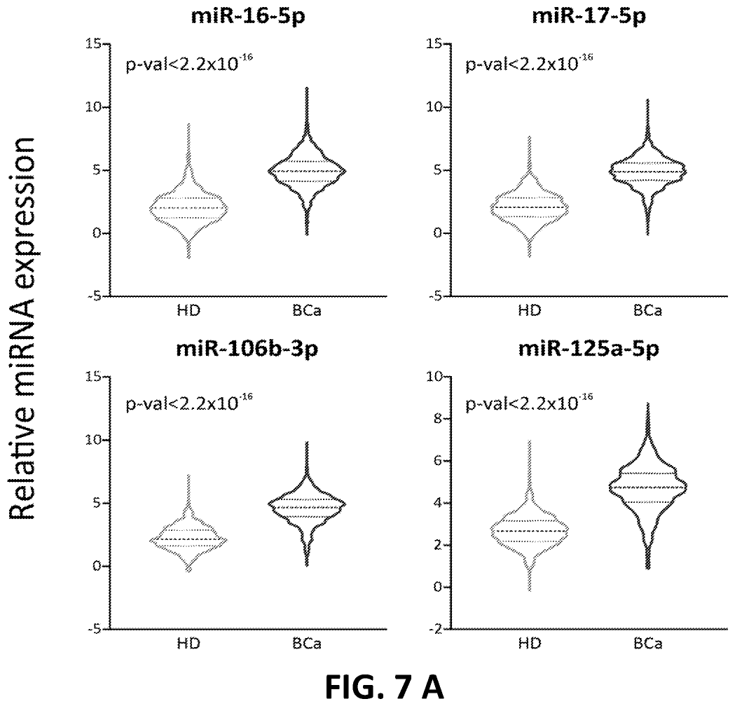

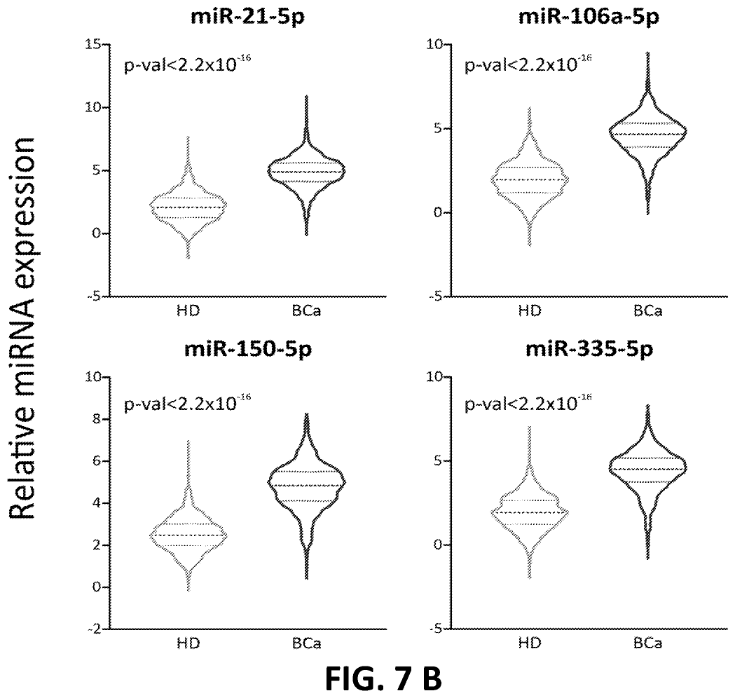

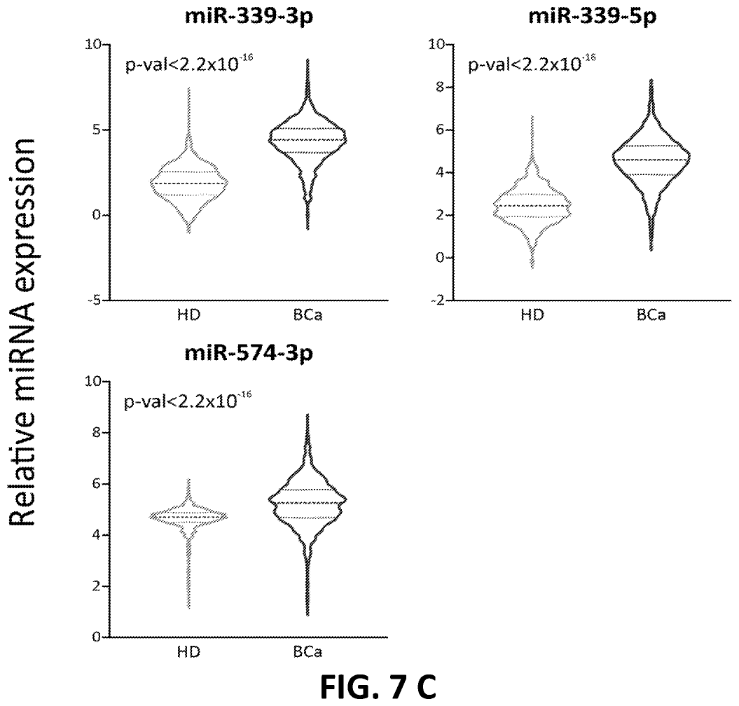

Finally, given that the analysis previously carried out with both cohorts (exploratory and validation) were in patients from different hospitals within Argentina, and in particular in CABA and GBA, without contemplating too much heterogeneity, it was proposed to verify that the miRNAs found would also be increased in cohorts of patients with BC and HD in other countries, with different environments, diet, etc. For this, expression microarray, data were obtained from the serum of 1272 patients with BC and 1272 VS from the National Cancer Center Hospital (NCCH) in Japan (Shimomura et al. 2016). Once the expression data was obtained, we proceeded to analyze the expression of the 11 candidate miRNAs in this new cohort, which was called in-silico external validation. Remarkably, all 11 miRNAs were found to be significantly increased in circulation from patients with BC relative to HD as observed in the validation cohort (FIGS. 7A, 7B, 7C).

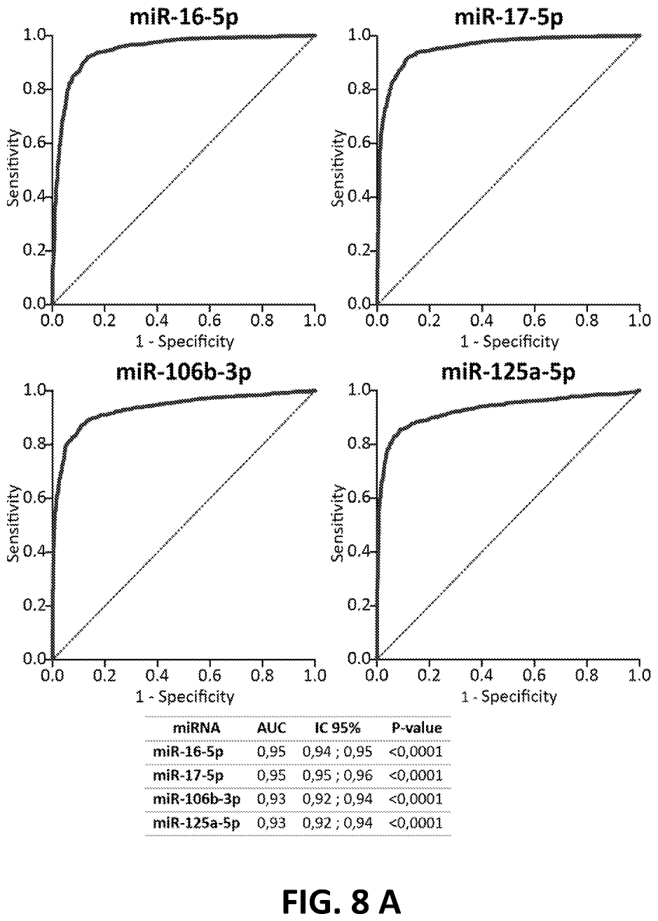

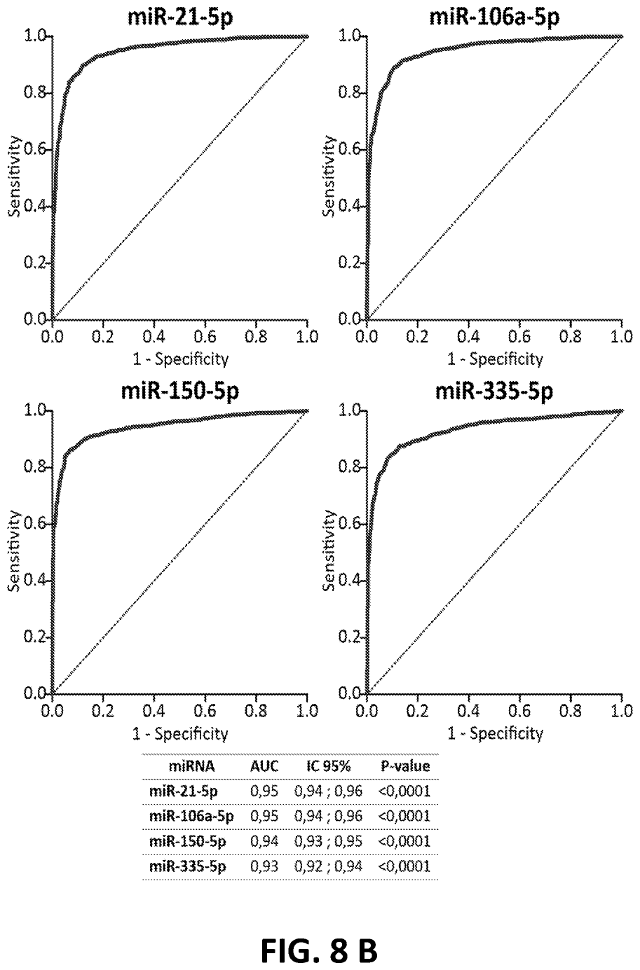

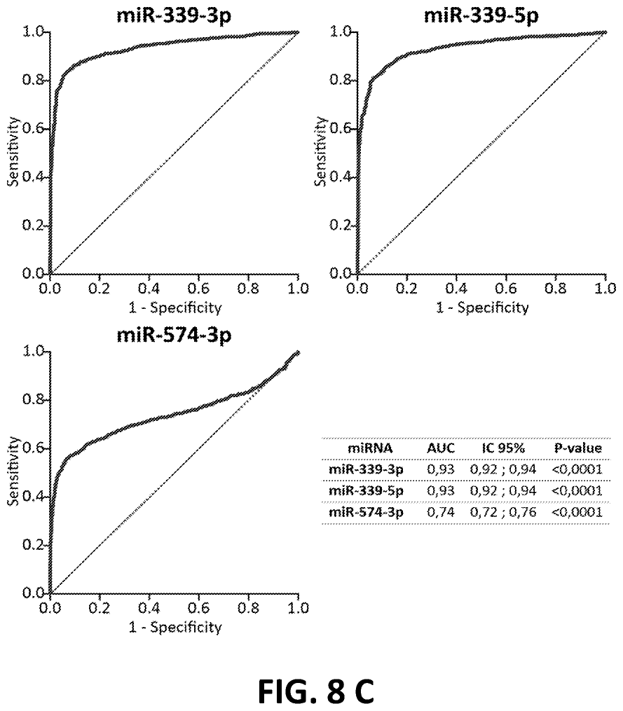

Just as the ROC curves were calculated in the two previous cohorts, in this external validation cohort they were also calculated using the expression data of the microarrays for each of the candidate miRNAs. It was found that the 11 miRNAs were excellent biomarkers since they all presented AUC values above 0.9, except for miR-574-3p, which presented an AUC of 0.7, being considered an acceptable biomarker (FIGS. 8A, 8B, 8C).

In this way, concluded that, from the use of microarrays of expression and sequencing of miRNAs, and through validation by RT-qPCR from plasma and serum samples of patients with BC and HD, 11 miRNAs were identified that could be used in the clinic as biomarkers of the disease.

### Statistical Analysis and Graphic Representation of the Results

The different experiments were analyzed as indicated in each case using the programs GraphPad 8.0.1 and RStudio 1.4.1106. Statistical significance was 5% in all cases. Statistical analyzes were performed using R software version 4.0.5 using the libraries listed below. The graphs represent the average of the experiments carried out with their standard deviation normalized to the control or the distribution of the samples using boxplots, plotting the median and the interquartile ranges, depending on what will facilitate understanding in each case. To evaluate normality and homogeneity of variances, Shapiro-Wilks and Levene's test or F test were used, respectively. When the assumptions were met, the T-Student test was used, and when they were not met, the Mann-Whitney, Wilcoxon or the median test were used.

### Example 3

### Development, Selection and Comparison of Predictive Models for the Early Detection of Breast Cancer

A predictive model for BC detection was established to be used for clinical BC screening. To do this, different machine learning tools were used to establish the model that best differentiates between patients with BC and healthy individuals, using the log 10 expression of certain miRNAs in plasma measured by RT-qPCR as explanatory variables. For this purpose, the Random-Forest and Lasso Regression variable selection techniques were used, based on the use of mathematical algorithms that allow automatically establishing relevant variables to be used in the construction of predictive models. In turn, those that were significant in the various models chosen were also taken into account for the selection of variables. Logistic regression combined with leave one out cross validation (LOOCV) was also used as a method for building the predictive model. Then, to choose the optimal model, the metrics of each one and the number of miRNAs included in each model were compared, and the one with the highest sensitivity and the lowest number of miRNAs was chosen. Finally, the combination of miRNAs chosen in various types of cancer was analyzed to establish whether this combination was specific for the detection of BC or if it could be useful in the detection of other types of cancer.

3.1 Selection of a Set of miRNAs for the Early Detection of BC

When making a predictive model, the first thing to do is analyze the data with which you are going to work (input data; training data), and try to make it as homogeneous as possible. In the present work, the variables to be used for choosing the optimal model were the plasma expression of the 11 miRNAs measured by RT-qPCR transformed with Log 10, in order to guarantee that data behave normally. Then, using the Lasso Regression and Random Forest automated variable selection techniques, different combinations of miRNAs were selected to be modeled later. Since the algebraic background inherent in the different mathematical algorithms is not the focus of this specification, different concepts of machine learning are introduced in a simplified way to allow the reader to correctly understand the techniques used until the selection of the final model. Unless specifically indicated, the terms used have the meanings used in the art.

3.2 Machine Learning Techniques Used in the Selection of miRNAs

### 3.2.1 Lasso Regression:

The Lasso Regression technique is based on a mathematical model that automatically penalizes those variables that are less relevant to the model or that do not provide new information, in order to eliminate them. This allows choosing those variables that have “survived” the selection technique objectively. The coefficient that Lasso uses to penalize is called the Lambda, and as its value grows, the number of surviving variables decreases.

To use this selection technique in the present work, the expression of the 11 candidate miRNAs transformed as mentioned above was used and, using the R software, the analysis was carried out. As a result of the application of the algorithm, the program returned a series of graphs that accounted for the selection that was made. What was observed in FIG. 9 was, on the one hand, the representation of each of the 11 miRNAs with a determined color line. Then, the way in which each of the miRNAs was penalized is observed as the corresponding lines disappear as the X axis is advanced from left to right (FIG. 9).

In the upper part of the graph (FIG. 9), the numbers that were observed are the amount of miRNAs that were surviving at each point, and denote that, as the X axis advanced, these numbers decrease. In the lower part of the graph (FIG. 9), the value of the Lambda logarithm was observed, and it can be seen how, as it growed, the miRNAs disappeared until finally all take a value of zero.

Lasso regression then made it possible to automatically define the optimal number of miRNAs to include in the model, and which miRNAs they were. In FIG. 10 it can be seen that 8 miRNAs were defined as the optimal number, a value that was defined in the upper part of the graph delimited by the lines in bold. In particular, the 8 miRNAs selected by this method were: miR-150-5p, miR-106a-5p, miR-125a-5p, miR-17-5p, miR-339-5p, miR-335-5p and miR-16-5p.

### 3.2.2 Random Forest

Another technique that was used in the selection of miRNAs to be used in predictive models was Random Forest. It consists of an algorithm that, through decision trees, results in a ranking of variables, from the most important to the least important according to this algorithm, while determining nodes or jumps in the importance of the variables, which allow make a selection of variables clearer. In particular in this work, the ranking obtained by Random Forest is detailed in FIG. 9. In it, it can be seen how the miRNAs miR-150-5p, miR-16-5p, miR-106a-5p, miR-339-3p and miR-339-5p were classified in the ranking as the 5 most important according to the order of appearance and a jump was established between these and the following miRNAs, demonstrated with the change in the MeanDecreaseAccuracy value associated with each miRNA.

In conclusion, using machine learning techniques, two sub-groups of particular miRNAs were established. The first, made up of the miRNAs miR-150-5p, miR-106a-5p, miR-125a-5p, miR-17-5p, miR-339-5p, miR-335-5p and miR-16-5p identified using Lasso Regression, and a second group, made up of the miRNAs miR-150-5p, miR-16-5p, miR-106a-5p, miR-339-3p and miR-339-5p defined by using Random Forest. These sets were then be used in the construction of predictive models for the early detection of breast cancer (BC).

### 3.3 Predictive Models

After establishing the subgroups of candidate miRNAs, the predictive models were built using the log 10 values of the expression of these circulating miRNAs measured by RT-qPCR in plasma from patients with BC or HD using machine learning. In particular, logistic regression was used, since it allows generating a model that gives as a result the probability of an individual having or not a certain condition or disease. This allowed different individuals to be classified as healthy or sick, taking into account the probability calculated for each one by the predictive model. Another important point to keep in mind is that the logistic regression also evaluated whether each of the variables (the miRNAs in this case) used in the model were relevant or not, with an associated p-value. Those that were not statistically significant were eliminated from the predictive model.

In turn, logistic regression was combined with the use of LOOCV. LOOCV is an algorithm that allows for more robust cross-validation using numbers of individuals that are not as large, as was the case in this case, and allowed for more reliable metrics to be obtained, a concept introduced later.

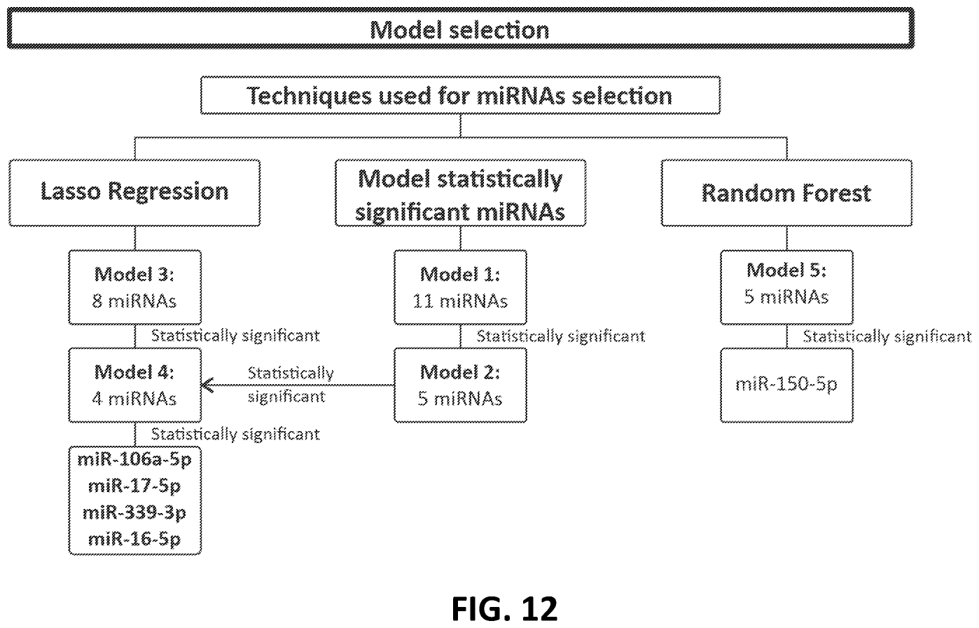

Initially, a total of 5 predictive models were built, as shown in FIG. 12. The first model (Model 1), was made up of the 11 candidate miRNAs; the second model (Model 2) was made up of 5 miRNAs (miR-106a-5p, miR-17-5p, miR-339-3p, miR-335-5p and miR-16-5p), those that were statistically significant in Model 1. Then, a model (Model 5) was built from the miRNAs identified by Random Forest (miR-150-5p, miR-16-5p, miR-106a-5p, miR-339-3p and miR-339-5p), where only miR-150-5p was significant. The model (Model 3) was also built using the miRNAs identified by Lasso Regression (miR-150-5p, miR-106a-5p, miR-125a-5p, miR-17-5p, miR-339-5p, miR-335-5p and miR-16-5p), which gave rise to a fifth model (Model 4), made up of 4 miRNAs (miR-106a-5p, miR-17-5p, miR-339-3p and miR-16-5p), which were the statistically significant ones of model 3 (which in turn were the same 4 significant miRNAs of Model 2). The final selection criterion was made by comparing the particular metrics of each model.

When choosing the best possible model, it was very important to define the criteria that were to be used for this purpose, which were based on the metric that was sought to highlight or maximize/minimize. Among the metrics that can be calculated from a model, the following were the most used:

Sensitivity: Percentage of sick individuals that are classified as positive by the model.

Specificity: Percentage of healthy individuals that are classified as negative by the model.

AUC ROC: Area under the ROC curve that combines sensitivity and specificity data to determine the cut-off point.

Accuracy: Percentage of individuals (sick and healthy) that were well classified by the model of the total number of individuals analyzed.

Positive predictive value: Percentage of true patients within those who were classified as positive by the model.

Negative predictive value: Percentage of true healthy within those who were classified as negative by the model.

False positive rate: Percentage of individuals who were classified by the model as sick while being healthy.

If, for example, the aim was to “not miss” any potentially sick individual, as is our case, the model with the greatest sensitivity would be sought, since it is possible to carry out another analysis to rule out the disease rather than under-diagnose it or, in other cases, words, have a slightly higher rate of false positives than false negatives. On the other hand, if the aim was to rule out a disease, the test or model to be used should have a high specificity. Other interesting metrics are Accuracy and positive predictive value, since both account for the performance of the model. They assess how well both sick and healthy were classified using the model and, on the other hand, how well really sick individuals were classified as positive, respectively. In turn, when evaluating the negative predictive value, this parameter also speaks of how well the model works, knowing the proportion of individuals classified as negative within those healthy. Finally, the AUC ROC combines both the specificity and the sensitivity as mentioned above, and, in turn, they are very useful to determine the cut-off point or threshold that will be used to classify individuals as healthy or sick. This cut-off point is a probability value, such as the one calculated for each individual in the model, and it is established that, if the value of the individual is above the cut-off point, it will be classified as sick, and if it is below, as healthy.

For the present invention, since it was sought to generate a predictive model for early detection, and in particular to be used in the future in a first screening in the clinic, it was very important that the model had the highest possible sensitivity. Another important point was the amount of miRNAs finally used in this model, since, by minimizing the number of miRNAs, future implementation in the clinic could be of lower cost, since a smaller number of miRNAs should be measured in the plasma of patients.

In addition to the 5 models disclosed above (Models 1 to Model 5), another two models (Model 6 and Model 7) were generated. Model 6 comprised miR106a-5p, miR 17-5p, miR16-05p, miR150-5p, miR125a-5p, miR339-5p, miR315-5p, miR21-5p, miR574-3p, and 106b-3p. Model 7 comprised miR106a-5p, miR17-5p, miR16-5p, miR150-5p, miR125a-5p, miR339-5p and miR335-5p.

After making all the predictive models using the previously described machine learning techniques, the metrics associated with each one were calculated and listed in TABLE 3. TABLE 3 presents all the metrics of the models named from 1 to 7 and the names of the miRNAs involved in each model. The chosen model was Model 7 (comprised of the miRNAs: miR-106a-5p, miR-125a-5p, miR-150-5p, miR-17-5p, miR-339-5p, miR-335-5p and miR-16-5p) since it was the one that showed the highest sensitivity of the 7, with a value of 90%, but also in our first pilot study could detect volunteers with breast cancer. Model 4 had a specificity of 71%, an Accuracy value of 83%, a precision of 81%, a negative predictive value of 87% and a false positive rate of 29%. The value of the AUC ROC was similar in all the models, which showed that, although the best model in terms of sensitivity was Model 4, all of them, to a greater or lesser extent, are good predictive models for BC. Models 4 and 7 were preliminarily selected for future development.

The seven models were evaluated in 50 volunteers recruited in a pilot study carried out at two Argentinean hospitals. Results showed that among 9 volunteers with inconclusive BiRad using mammogram (BiRad 0), one was positive for Model 3 and for Model 7. Importantly, after patient follow up and other clinical studies it was found that this volunteer in fact had breast cancer. Additionally, the application of Model 7 to the 50 volunteers indicated that 20% of them were positive (i.e., they were predicted to have breast cancer), in comparison with 16% for Model 1, 18% for Model 3; 2% for Model 4, and 6% for Model 6.

TABLE 3
Summary of the miRNAs included and the metrics obtained from the predictive models.

| | | | | | | POSITIVE | NEGATIVE | FALSE |
|---|---|---|---|---|---|---|---|---|
| | | Sensitivity | Specificity | | ACCURACY | PREDECTIVE | PREDECTIVE | POSITIVE |
| MODELS | INCLUDED miRNAs | (%) | (%) | AUCROC | (%) | VALUE (%) | VALUE (%) | RATE (%) |
| 1 | 150-5p, 106b-3p, 106a-5p, | 87 | 73 | 0.88 | 81 | 81 | 80 | 23 |
| | 125a-5p, 17-5p, 574-3p, | | | | | | | |
| | 339-5p, 339-3p, 335-5p, | | | | | | | |
| | 16-5p, 21-5p | | | | | | | |
| 2 | 106a-5p, 17-5p, 339-3p, | 88 | 77 | 0.89 | 83 | 84 | 82 | 23 |
| | 335-5p, 16-5p | | | | | | | |
| 3 | 150-5p, 106a-5p, 125a-5p, | 77 | 86 | 0.89 | 81 | 89 | 73 | 14 |
| | 17-5p, 339-5p, 339-3p, | | | | | | | |
| | 335-5p, 16-5p | | | | | | | |
| 4 | 106a-5p, 17-5p, 339-3p, | 92 | 71 | 0.89 | 83 | 81 | 87 | 29 |
| | 16-5p | | | | | | | |
| 5 | 150-5p, 106a-5p, 339-5p, | 85 | 66 | 0.8 | 77 | 77 | 76 | 34 |
| | 339-3p, 16-5p | | | | | | | |
| 6 | 106a-5p, 17-5p, 16-5p, | 87 | 75 | 0.87 | 82 | 83 | 81 | 25 |
| | 150-5p, 125a-5p, 339-5p, | | | | | | | |
| | 335-5p, 21-5p, 574-3p, | | | | | | | |
| | 106b-3p | | | | | | | |
| 7 | 106a-5p, 17-5p, 16-5p, | 90 | 73 | 0.88 | 83 | 82 | 84 | 27 |
| | 150-5p, 125a-5p, 339-5p, | | | | | | | |
| | 335-5p | | | | | | | |

In summary, through the use of machine learning, 7 predictive models were built using the log 10 of the expression of circulating miRNAs from the plasma of patients with BC and HD, including different combinations of miRNAs. The corresponding metrics were measured to measure the performance of the models, indicating that the best performing model was Model 7.

3.4 Calculation of the Probability of Suffering from BC Based on the Five Predictive Models.

To obtain the probability, in the present invention a statistical model called logistic regression associated with a cross validation of machine learning (Cross Validation Leave One Out) was used. To do this, a value (3 coefficient) must first be obtained for each miRNA, which will then be informed within the equation to obtain a probability value. This value will be compared with the threshold value or cut-off point, which will serve to classify the individual as healthy or sick.

Other statistical analysis techniques can be used, which must be fine-tuned and validated, taking into account that for any of them, the expression level of the set or group of miRNAs (predictive models) will be the basis for calculating the negative control (healthy patients) or cut-off value; and where those who exceed said cut-off or control value will be considered sick patients.

The logistic regression technique for calculating the probability that an individual has BC, for each of the groups of miRNAs (predictive models), is detailed below.

Predictive Model 1: For the predictive Model 1 of TABLE 3.
(i) averaging the Ct values obtained from the qPCR for each of the 11 specific miRNAs (miR-150-5p, miR-106b-3p, miR-106a-5p, miR-125a-5p, miR-17-5p, miR-574-3p, miR-339-5p, miR-339-3p, miR-335-5p, miR-16-5p and miR-21-5p);
(ii) subtracting the average value of each of the miRNAs from the Ct value obtained from the control (cel-miR-39-3p): Ct control−Ct miR_{specific}=ΔCtmiR_{specific};
squaring the ΔCtmiR_{specific}: 2\hat{ }(ΔCtmiR_{specific})=Value×miR_{specific};
calculating the logarithm in base e of Value×miR_{specific}:

$$Ln ( Value \times {miR}_{specific} ) = Result {miR}_{specific ,}$$

(iii) which results in 4 values per individual: Value A (miR₁₅₀₋₅ₚ Result), Value B: (miR_{106b-3p} Result), Value C (miR₁₀₆ₐ₋₅ₚ Result), Value D (miR₁₂₅ₐ₋₅ₚ Result); Value E (miR₁₇₋₅ₚ Result), Value F (miR₅₇₄₋₃ₚ Result); Value G (miR₃₃₉₋₅ₚ Result), Value H (miR₃₃₉₋₃ₚ Result), Value I (miR₃₃₅₋₅ₚ Result); Value J (miR₁₆₋₅ₚ Result), Value K (miR₂₁₋₅ₚ Result);
(iv) calculating the probability of having BC for each individual by integrating the 11 results of the miRNAs in the following equation: p(x)=1/(e\hat{ }(−(β₀+β₁*ValueA+β₂*ValueB+β₃*ValueC+β₄*ValueD+β₅*ValueE+β₆*ValueF+β₇*ValueG+β₈*ValueH+β₉*ValueI+β₁₀*ValueJ+β₁₁*ValueK))+1), wherein the values of the beta coefficients are the following: β₀=2.6258, β₁=0.3280, β₂=−0.990, β₃=1.2630, ₄=−0.6357, β₅=−2.6589, β₆=0.5139, β₇=−0.1197, β₈=2.3412, β₉=−1.0167, β₁₀=1.6683, β₁₁=0.3948
(v) comparing the obtained probability score (p(x)) with the specific cut-off point for this predictive model, which is 0.39;
wherein if the value of the individual's probability of having BC was equal to or greater than 0.39, the individual was classified as sick (i.e., it was predicted to have BC), and if it was less than 0.39, it was classified as healthy.

Predictive Model 2: For the predictive Model 2 of TABLE 3.
(i) averaging the Ct values obtained from the qPCR for each of the 5 specific miRNAs Averaging the Ct values obtained from the qPCR for each miRNA separately (miR106a-5p, miR17-5p, miR339-3p, miR16-5p, miR335-5p);
(ii) subtracting the average value of each of the miRNAs from the Ct value obtained from the control (cel-miR-39-3p): Ct control−Ct miR_{specific}=ΔCtmiR_{specific};
(iii) squaring the ΔCtmiR_{specific}: 2\hat{ }(ΔCtmiR_{specific})=Value×miR_{specific};
(iv) calculating the logarithm in base e of Value×miR_{specific}: Ln(Value×miR_{specific})=Result miR_{specific},
which results in 5 values per individual: Value A (miR₁₀₆ₐ₋₅ₚ Result), Value B (miR₁₇₋₅ₚ Result), Value C (miR₃₃₉₋₃ₚ Result), Value D (miR₁₆₋₅ₚ Result), Value E (miR₃₃₅₋₅ₚ Result);
(v) calculating the probability of having BC for each individual by integrating the 5 results of the miRNAs in the following equation: p(x)=1/(e\hat{ }(−(β₀+β₁*Value A+β₂*Value B+β₃*Value C+β₄*Value D+β₅*Value E))+1), wherein the values (vi) of the beta coefficients are the following: β₀=3.9420, β₁=1.0664, β₂=−2.8282, β₃=1.8165, β₄=1.9203, β₅=−0.824
(vii) comparing the obtained probability score (p(x)) with the specific cut-off point for this predictive model, which is 0.4432;
wherein if the value of the individual's probability of having BC was equal to or greater than 0.39, the individual was classified as sick, and if it was less than 0.4432, it was classified as healthy.

Predictive Model 3: For the predictive Model 3 of TABLE 3.
(i) averaging the Ct values
(ii) obtained from the qPCR for each of the 8 specific miRNAs Averaging the Ct values obtained from the qPCR for each miRNA separately (miR150-5p, miR106a-5p, miR125a-5p, miR17-5p, miR339-5p, miR339-3p, miR335-5p, miR16-5p);
(iii) subtracting the average value of each of the miRNAs from the Ct value obtained from the control (cel-miR-39-3p): Ct control−Ct miR_{specific}=ΔCtmiR_{specific}; (iv) squaring the ΔCtmiR_{specific}: 2\hat{ }(ΔCtmiR_{specific})=Value×miR_{specific};
(v) calculating the logarithm in base e of Value×miR_{specific}: Ln(Value×miR_{specific})=Result miR_{specific},
which results in 8 values per individual: Value A (miR₁₅₀₋₅ₚ Result), Value B (miR₁₀₆ₐ₋₅ₚ Result), Value C (miR₁₂₅ₐ₋₅ₚ Result), Value D (miR₁₇₋₅ₚ Result), Value E (miR₃₃₉₋₅ₚ Result), Value F (miR₃₃₉₋₃ₚ Result), Value G (miR₃₃₅₋₅ₚ Result), Value H (miR₁₆₋₅ₚ Result);
(vi) calculating the probability of having BC for each individual by integrating the 8 results of the miRNAs in the following equation: p(x)=1/(e\hat{ }(−(β₀+β₁*Value A+β₂*Value B+β₃*Value C+β₄*Value D+β₅*Value E+β₆*Value F+β₇*Value G+β₈*Value H))+1), wherein the values of the beta coefficients are the following:
β₀=4.0444, β₁=0.2895, β₂=1.1846, β₃=−0.6834, β₄=−2.5341, β₅=−0.1790, β₆=2.2036, β₇=−0.9407, β₈=1.7400
(vii) comparing the obtained probability score (p(x)) with the specific cut-off point for this predictive model, which is 0.6217;
wherein if the value of the individual's probability of having BC was equal to or greater than 0.6217, the individual was classified as sick, and if it was less than 0.6217, it was classified as healthy.

Predictive Model 4: Predictive Model 4 disclosed in TABLE 3 comprises the following steps:
(i) averaging the Ct values obtained from the qPCR for each of the 4 specific miRNAs (miR-106a-5p, miR-17-5p, miR-339-3p and miR-16-5p);
(ii) subtracting the average value of each of the miRNAs from the Ct value obtained from the control (cel-miR-39-3p): Ct control−Ct miR_{specific}=ΔCtmiR_{specific};
(iii) squaring the ΔCtmiR_{specific}: 2\hat{ }(ΔCtmiR_{specific})=Value×miR_{specific};
(iv) calculating the logarithm in base e of Value×miR_{specific}:

$$Ln ( Value \times {miR}_{specific} ) = Result {miR}_{specific ,}$$

which results in 4 values per individual: Value A (miR₁₀₆ₐ₋₅ₚ Result), Value B (miR₁₇₋₅ₚ Result), Value C (miR₃₃₉₋₃ₚ Result) and Value D (miR₁₆₋₅ₚ Result);
(v) calculating the probability of having BC for each individual by integrating the 4 results of the miRNAs in the following equation:

$$p ( x ) = 1 / ( e \hat{} ( - ( {β}_{0} + {β}_{1} * Value A + {β}_{2} * Value B + {β}_{3} * Value C + {β}_{4} * Value D ) ) + 1 )$$

wherein the values
of the beta coefficients are:
β₀=5.9446; β₁=1.1062; β₂=−3.5628; β₃=1.5886; β₄=1.9661; and,
(vi) comparing the obtained probability score (p(x)) with the specific cut-off point for this predictive model, which is 0.3744;
wherein if the value of the individual's probability of having BC was equal to or greater than 0.3744, the individual was classified as sick, and if it was less than 0.3744, it was classified as healthy.

Predictive Model 5: For the predictive Model 5 of TABLE 3.
(i) averaging the Ct values obtained from the qPCR for each of the 5 specific miRNAs Averaging the Ct values obtained from the qPCR for each miRNA separately (miR150-5p, miR106a-5p, miR339-5p, miR339-3p, miR16-5);
(ii) subtracting the average value of each of the miRNAs from the Ct value obtained from the control (cel-miR-39-3p): Ct control−Ct miR_{specific}=ΔCtmiR_{specific};
(iii) squaring the ΔCtmiR_{specific}: 2\hat{ }(ΔCtmiR_{specific})=Value×miR_{specific};
(iv) calculating the logarithm in base e of Value×miR_{specific}: Ln(Value×miR_{specific})=Result miR_{specific},
which results in 5 values per individual: Value A (miR₁₅₀₋₅ₚ Result), Value B (miR₁₀₆ₐ₋₅ₚ Result), Value C (miR₃₃₉₋₅ₚ Result), Value D (miR₃₃₉₋₃ₚ Result), Value E (miR₁₆₋₅ Result);
(v) calculating the probability of having BC for each individual by integrating the 5 results of the miRNAs in the following equation: p(x)=1/(e\hat{ }(−(β₀+β₁*Value A+β₂*Value B+β₃*Value C+β₄*Value D+β₅*Value E))+1), wherein the values of the beta coefficients are the following: β₀=2.8464, β₁=0.6173, β₂=−0.3038, β₃=0.5280, β₄=−0.3079, β₅=0.4969
(vi) comparing the obtained probability score (p(x)) with the specific cut-off point for this predictive model, which is 0.4905;
wherein if the value of the individual's probability of having BC was equal to or greater than 0.4905, the individual was classified as sick, and if it was less than 0.4905, it was classified as healthy.

Predictive Model 6: For the predictive Model 6 of TABLE 3.
(i) averaging the Ct values obtained from the qPCR for each of the 10 specific miRNAs Averaging the Ct values obtained from the qPCR for each miRNA separately (miR-150-5p, miR-106b-3p, miR-106a-5p, miR-125a-5p, miR-17-5p, miR-574-3p, miR-339-5p, miR-335-5p, miR-16-5p and miR-21-5p);
(ii) subtracting the average value of each of the miRNAs from the Ct value obtained from the control (cel-miR-39-3p): Ct control−Ct miR_{specific}=ΔCtmiR_{specific};
squaring the ΔCtmiR_{specific}: 2\hat{ }(ΔCtmiR_{specific})=Value×miR_{specific};
(iii) calculating the logarithm in base e of Value×miR_{specific}: Ln(Value×miR_{specific})=Result miR_{specific},
which results in 10 values per individual: Value A (miR₁₅₀₋₅ₚ Result), Value B (miR_{106b-3p} Result), Value C (miR₁₀₆ₐ₋₅ₚ Result), Value D (miR₁₂₅ₐ₋₅ₚ Result), Value E (miR₁₇₋₅ₚ Result), Value F (miR₅₇₄₋₃ₚ Result), Value G (miR₃₃₉₋₅ₚ Result), Value H (miR₃₃₅₋₅ₚ Result), Value I (miR₁₆₋₅ₚ Result), Value J (miR₂₁₋₅ₚ Result);
(iv) calculating the probability of having BC for each individual by integrating the 10 results of the miRNAs in the following equation: p(x)=1/(e\hat{ }(−(β₀+β₀*ValueA+β₂*ValueB+β₃*ValueC+β₄*ValueD+β₅*ValueE+β₆*ValueF+δ₇*ValueG+β₈*ValueH+β₉*ValueI+β₁₀*ValueJ))+1), wherein the values of the beta coefficients are the following: β₀=−2.9205, β₁=0.4202, β₂=−0.7059, β₃=1.0565, β₄=−0.7592, β₅=−2.2015, β₆=0.4475, β₇=0.9130, β₈=−0.8363, β₉=1.8845, β₁₀=0.8935
(v) comparing the obtained probability score (p(x)) with the specific cut-off point for this predictive model, which is 0.4404;
wherein if the value of the individual's probability of having BC was equal to or greater than 0.4404, the individual was classified as sick, and if it was less than 0.4404, it was classified as healthy.

Predictive Model 7: For the predictive Model 7 of TABLE 3.
(i) averaging the Ct values obtained from the qPCR for each of the 7 specific miRNAs Averaging the Ct values obtained from the qPCR for each miRNA separately (miR-106a-5p, miR-125a-5p, miR-150-5p, miR-17-5p, miR-339-5p, miR-335-5p and miR-16-5p.);
(ii) subtracting the average value of each of the miRNAs from the Ct value obtained from the control cel-miR-39-3: Ct control−Ct miR_{specific}=ΔCtmiR_{specific};

$$squaring the Δ {CtmiR}_{specific} : 2 \hat{} ( Δ {CtmiR}_{specific} ) = Value \times {miR}_{specific} ;$$

(iii) calculating the logarithm in base e of Value×miR_{specific}: Ln(Value×miR_{specific})=Result miR_{specific},
which results in 7 values per individual: Value A (miR₁₅₀₋₅ₚ Result), Value B (miR₁₀₆ₐ₋₅ₚ Result), Value C (miR₁₂₅ₐ₋₅ₚ Result), Value D (miR₁₇₋₅ₚ Result), Value E (miR₃₃₉₋₅ₚ Result), Value F (miR₃₃₅₋₅ₚ Result), Value G (miR₁₆₋₅ₚ Result);
(v) calculating the probability of having BC for each individual by integrating the 7 results of the miRNAs in the following equation: p(x)=1/(e\hat{ }(−(β₀+β₁*Value A+β₂*Value B+β₃*Value C+β₄*Value D+β₅*Value E+β₆*Value F+β₇*Value G))+1), wherein the values of the beta coefficients are the following: β₀=−1.7792, β₁=0.4219, β₂=1.0883, β₃=−0.7773, β₄=−1.8923, β₅=0.9227, β₆=−0.71850.4969, β₇=2.0524
(vi) comparing the obtained probability score (p(x)) with the specific cut-off point for this predictive model, which is 0.3724;
wherein if the value of the individual's probability of having BC was equal to or greater than 0.3724, the individual was classified as sick, and if it was less than 0.3724, it was classified as healthy.

### Example 4

### Experimental Trial for Clinical Phase Tests.

A pilot test is ongoing at the Hospital Posadas and the Hospital Militar Central, where women between 50 and 70 years old (n=500) are enrolled who attend their gynecological check-up without a history of previous oncological disease and, after informed consent signature, they are asked for a blood sample along with routine studies such as mammograms and/or breast ultrasound. In these blood samples the four miRNAs used in Model 4 are measured: miR-106a-5p, miR-17-5p, miR-339-3p and miR-16-5p, the algorithm obtained by logistic regression will be applied to obtain a probability value of having BC. In summary, in this pilot test, the combination of liquid biopsies added to conventional images will allow establishing how early the BC detection is to support the use of liquid biopsies as a routine screening method.

### Example 5

### Kit for Detection of BC

One way of implementing the BC detection method based on liquid biopsies (miRNAs) of the present invention is based on a kit comprising:
(1) Specific primers for the reverse transcription stage of the 4 miRNAs used to detect breast cancer using Model 4:

hsa-miR-16-5p:
(SEQ ID NO: 26)
GTCTCCTCTGGTGCAGGGTCCGAGGTATTCGCACCAGAGGAGACCGCCAA
hsa-miR-17-5p:
(SEQ ID NO: 20)
GTCTCCTCTGGTGCAGGGTCCGAGGTATTCGCACCAGAGGAGACCTACCT
hsa-miR-106a-5p:
(SEQ ID NO: 16)
GTCTCCTCTGGTGCAGGGTCCGAGGTATTCGCACCAGAGGAGACCTACCT
hsa-miR-339-3p:
(SEQ ID NO: 30)
GTCTCCTCTGGTGCAGGGTCCGAGGTATTCGCACCAGAGGAGACCGGCTC

(2) Specific primers for the qPCR step

```
  (SEQ ID NO: 27)
hsa-miR-16-5p: GGCCCGTAGCAGCACGTAAATA
  (SEQ ID NO: 21)
hsa-miR-17-5p: GGATGGCAAAGTGCTTACAGTGC
  (SEQ ID NO: 17)
hsa-miR-106a-5p: GGCGGAAAAGTGCTTACAGTGC
  (SEQ ID NO: 31)
hsa-miR-339-3p: GGTGAGCGCCTCGACGAC
(SEQ ID NO: 38)
Primer Universal Rv: TGTGTGCAGGGTCCGAGGTATT
```

(3) Optionally, specific primers for the cel39 control for the reverse transcription step (sequence) and for the qPCR step

cel-miR-39-3p for RT:
(SEQ ID NO: 36)
GTCTCCTCTGGTGCAGGGTCCGAGGTATTCGCACCAGAGGAGACCAAGCT
cel-miR-39-3p for qPCR:
(SEQ ID NO: 37)
CGGGGTCACCGGGTGTAAATC

(4) Optionally, synthetic positive controls for qPCR step

```
hsa-miR-16-5p:
  (SEQ ID NO: 26)
GTCTCCTCTGGTGCAGGGTCCGAGGTATTCGCACCAGAGGAGACCGCCA
ATATTTACGTGCTGCTA
hsa-miR-17-5p:
  (SEQ ID NO: 20)
GTCTCCTCTGGTGCAGGGTCCGAGGTATTCGCACCAGAGGAGACCTACC
TGCACTGTAAGCACTTTG
hsa-miR-106a-5p:
  (SEQ ID NO: 16)
GTCTCCTCTGGTGCAGGGTCCGAGGTATTCGCACCAGAGGAGACCTACC
TGCACTGTAAGCACTTTT
hsa-miR-339-3p:
  (SEQ ID NO: 30)
GTCTCCTCTGGTGCAGGGTCCGAGGTATTCGCACCAGAGGAGACCGGCT
CTGTCGTCGAGGCGCTCA
cel-miR-39-3p:
(SEQ ID NO: 36)
GTCTCCTCTGGTGCAGGGTCCGAGGTATTCGCACCAGAGGAGACCAAGC
TGATTTACACCCGGTGA
```

and
(5) optionally, a procedures manual.

The same approach disclosed above for Model 4 can be used to implement the BC detection method based on liquid biopsies (miRNAs) of the present invention as a kit specific for Model 1, Model 2, Model 3, Model 5, Model 6 or Model 7.

### Example 6

### Clinical Trials

Women within a suitable age range (e.g., 20 to 40, 30 to 45, 35 to 50, 50 to 70 years, etc) will be enrolled who attend their gynecological check-up without a history of previous oncological disease and, after informed consent signature, they will be asked for one or more blood samples along with routine studies such as mammograms and/or breast ultrasounds.

In these blood samples, the miRNAs used in Model 1, Model 2, Model 3, Model 4, Model 5, Model 6, or Model 7 will be measured. Alternatively, the full panel of miRNAs disclosed in the present application (miR-150-5p, miR-106b-3p, miR-106a-5p, miR-125a-5p, miR-17-5p, miR-574-3p, miR-339-5p, miR-339-3p, miR-335-5p, miR-16-5p, and miR-21-5p) or a subset thereof will be measured.

One or more machine learning algorithms based on the disclosed miRNA gene panels (e.g., Models 1 to 7 disclosed herein) will be applied to the measured miRNA levels to obtain a probability value of having breast cancer according to the selected Model or a combination thereof. The application of the disclosed Model or combination thereof to miRNA expression levels in blood samples, alone or in combination with biopsies (e.g., solid or liquid biopsies) and/or other detection methods (e.g., mammograms, ultrasound, breast magnetic resonance imaging, etc.) will allow establishing whether the subject has breast cancer or is at risk of developing breast cancer.

The determination of a probability of having breast cancer according to the models disclosed herein will indicate how early the detection of breast cancer is possible. Furthermore, it will support the use of liquid biopsies and miRNA-based detection methods based on machine learning applied to the full panel of miRNAs disclosed herein and subsets thereof as described in the present application as routine screening methods for breast cancer detection.

SEQUENCES

| SEQ | | |
|---|---|---|
| ID | | |
| NO | Short description | Sequence |
| 1 | hsa-miR-150-5p | UCUCCCAACCCUUGUACCAGUG |
| | MIMAT0000451 (miRBase) | |
| | URS000016FD1A_9606 | |
| | (RNAcentral) | |
| 2 | hsa-miR-17-5p | CAAAGUGCUUACAGUGCAGGUAG |
| | MIMAT0000070 (miRBase) | |
| | URS00002075FA_9606 | |
| | (RNA central) | |
| 3 | bsa-miR-574-3p | CACGCUCAUGCACACACCCACA |
| | MIMAT0003239 (miRBase) | |
| | URS00001CF056_9606 | |
| | (RNA central) | |
| 4 | hsa-miR-125a-5p | UCCCUGAGACCCUUUAACCUGUGA |
| | MIMAT0000443 (miRBase) | |
| | URS00005A4DCF 9606 | |
| | (RNA central) | |
| 5 | hsa-miR-106b-3p | CCGCACUGUGGGUACUUGCUGC |
| | MIMAT0004672 (miRBase) | |
| | URS0000384021_9606 | |
| | (RNA central) | |
| 6 | bsa-miR-16-5p | UAGCAGCACGUAAAUAUUGGCG |
| | MIMAT0000069 (miRBase) | |
| | URS00004BCD9C_9606 | |
| | (RNA central) | |
| 7 | hsa-miR-21-5p | UAGCUUAUCAGACUGAUGUUGA |
| | MIMAT0000076 (miRBase) | |
| | URS000039ED8D_9606 | |
| | (RNA central) | |
| 8 | hsa-miR-106a-5p | AAAAGUGCUUACAGUGCAGGUAG |
| | MIMAT0000103 (miRBase) | |
| | URS00003FE4D4_9606 | |
| | (RNA central) | |
| 9 | hsa-miR-339-5p | UCCCUGUCCUCCAGGAGCUCACG |
| | MIMAT0000764 (miRBase) | |
| | URS000003FD55_9606 | |
| | (RNA central) | |
| 10 | hsa-miR-339-3p | UGAGCGCCUCGACGACAGAGCCG |
| | MIMAT0004702 (miRBase) | |
| | URS000055B190_9606 | |
| | (RNA central) | |
| 11 | bsa-miR-335-5p | UCAAGAGCAAUAACGAAAAAUGU |
| | MIMAT0000765 (miRBase) | |
| | URS0000237AF9_9606 | |
| | (RNA central) | |
| 12 | cel-miR-39-3p | UCACCGGGUGUAAAUCAGCUUG |
| | MIMAT0000010 (miRBase) | |
| | URS00005D4EC7_6239 | |
| | (RNA central) | |
| 13 | RT-Stem-loop-Rv | TGGTGCAGGGTCCGAGGTATT |
| 14 | RT-hsa-miR-21-5p-STEM | GTCTCCTCTGGTGCAGGGTCCGAGGTATTCGCACCA |
| | | GAGGAGACTCAACA |
| 15 | RT-hsa-miR-21-5p Fw | CGGGGGGTAGCTTATCAGACTG |
| 16 | RT-hsa-miR-106a-5p-STEM | GTCTCCTCTGGTGCAGGGTCCGAGGTATTCGCACCA |
| | | GAGGAGACCTACCT |
| 17 | RT-hsa-miR-106a-5p Fw | GGCGGAAAAGTGCTTACAGTGC |
| 18 | RT-hsa-miR-125a-5p-STEM | GTCTCCTCTGGTGCAGGGTCCGAGGTATTCGCACCA |
| | | GAGGAGACTCACAG |
| 19 | RT-hsa-miR-125a-5p Fw | CCCCCTCCCTGAGACCCTTTAAC |
| 20 | RT-hsa-miR-17-5p-STEM | GTCTCCTCTGGTGCAGGGTCCGAGGTATTCGCACCA |
| | | GAGGAGACCTACCT |
| 21 | RT-hsa-miR-17-5p Fw | GGATGGCAAAGTGCTTACAGTGC |
| 22 | RT-hsa-miR-106b-3p-STEM | GTCTCCTCTGGTGCAGGGTCCGAGGTATTCGCACCA |
| | | GAGGAGACGCAGCA |
| 23 | RT-hsa-miR-106b-3p Fw | GGGCCGCACTGTGGGTAC |
| 24 | RT-hsa-miR-150-5p-STEM | GTCTCCTCTGGTGCAGGGTCCGAGGTATTCGCACCA |
| | | GAGGAGACCACTGG |
| 25 | RT-hsa-miR-150-5p Fw | CCCCCTCTCCCAACCCTTGT |
| 26 | RT-hsa-miR-16-5p-STEM | GTCTCCTCTGGTGCAGGGTCCGAGGTATTCGCACCA |
| | | GAGGAGACCGCCAA |
| 27 | RT-hsa-miR-16-5p Fw | GGCCCGTAGCAGCACGTAAATA |
| 28 | RT-hsa-miR-335-5p-STEM | GTCTCCTCTGGTGCAGGGTCCGAGGTATTCGCACCA |
| | | GAGGAGACACATTT |
| 29 | RT-hsa-miR-335-5p Fw | CGGCGGTCAAGAGCAATAACG |
| 30 | RT-hsa-miR-339-3p-STEM | GTCTCCTCTGGTGCAGGGTCCGAGGTATTCGCACCA |
| | | GAGGAGACCGGCTC |
| 31 | RT-hsa-miR-339-3p Fw | GGTGAGCGCCTCGACGAC |
| 32 | RT-hsa-miR-339-5p-STEM | GTCTCCTCTGGTGCAGGGTCCGAGGTATTCGCACCA |
| | | GAGGAGACCGTGAG |
| 33 | RT-hsa-miR-339-5p Fw | CCCTCCCTGTCCTCCAGGAG |
| 34 | RT-hsa-miR-574-3p-STEM | GTCTCCTCTGGTGCAGGGTCCGAGGTATTCGCACCA |
| | | GAGGAGACTGTGGG |
| 35 | RT-hsa-miR-574-3p Fw | CGCCACGCTCATGCACAC |
| 36 | RT-cel-miR-39-3p-STEM | GTCTCCTCTGGTGCAGGGTCCGAGGTATTCGCACCA |
| | | GAGGAGACCAAGCT |
| 37 | RT-cel-miR-39-3p Fw | CGGGGTCACCGGGTGTAAATC |
| 38 | Primer Universal Rv | TGTGTGCAGGGTCCGAGGTATT |
| 39 | Linker sequence | GCGGCGG |
| 40 | Stem-loop sequence | GTCTCCTCTGGTGCAGGGTCCGAGGTATTCGCACCA |
| | | GAGGAGAC |

It is to be appreciated that the Detailed Description section, and not the Summary and Abstract sections, is intended to be used to interpret the claims. The Summary and Abstract sections may set forth one or more but not all exemplary embodiments of the present invention as contemplated by the inventor(s), and thus, are not intended to limit the present invention and the appended claims in any way.

The present invention has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific embodiments will so fully reveal the general nature of the invention that others can, by applying knowledge within the skill of the art, readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present invention should not be limited by any of the above-described exemplary embodiments, but should be defined only in accordance with the following claims and their equivalents.

Unless otherwise defined, all technical and scientific terms used herein have the same meaning as commonly understood by one of ordinary skill in the art. Although methods and materials similar or equivalent to those described herein can be used in the practice or testing of the present invention, suitable methods and materials are described herein.

All publications, patent applications, patents, and other references mentioned herein are incorporated by reference in their entirety. Database entries and electronic publications disclosed in the present disclosure are incorporated by reference in their entireties. The version of the database entry or electronic publication incorporated by reference in the present application is the most recent version of the database entry or electronic publication that was publicly available at the time the present application was filed. The database entries corresponding to gene or protein identifiers (e.g., genes or proteins identified by an accession number or database identifier of a public database such as Genbank, Refseq, or Uniprot) disclosed in the present application are incorporated by reference in their entireties. The gene or protein-related incorporated information is not limited to the sequence data contained in the database entry. The information incorporated by reference includes the entire contents of the database entry in the most recent version of the database that was publicly available at the time the present application was filed. In case of conflict, the present specification, including definitions, will control. In addition, the materials, methods, and examples are illustrative only and not intended to be limiting.

1.-64. (canceled)

65. A method for determining the breast cancer status in a subject in need thereof, comprising applying a machine-learning classifier to a plurality of miRNA expression levels obtained from a miRNA biomarker panel from a sample obtained from the subject, wherein the machine-learning classifier identifies the subject as having or not having breast cancer.

66. A method for treating a human subject afflicted with breast cancer comprising: (i) identifying, prior to the administration, a subject having or not having a specific breast cancer status by applying the method according of claim 65; and (ii) administering a breast cancer therapy to the subject.

67. A method for identifying a human subject afflicted with a breast cancer suitable for treatment with a breast cancer therapy, the method comprising applying the method according to claim 65, wherein the assignment of the sample to a specific breast cancer status, indicates that a specific breast cancer therapy can be administered to treat the cancer.

68. The method of claim 65, wherein the machine-learning classifier is a model obtained by Linear Regression, Random Forest, Logistic Regression, Artificial Neural Network (ANN), Support Vector Machine (SVM), XGBoost (XGB), glmnet, cforest, Classification and Regression Trees for Machine-learning (CART), treebag, K-Nearest Neighbors (kNN), or a combination thereof.

69. The method of claim 68, wherein the Linear Regression is Lasso Regression.

70. The method of claim 65, wherein the miRNA biomarker panel comprise 4, 5, 6, 7, 8, 9, 10, or 11 miRNAs selected from the group consisting of miR-150-5p, miR-106b-3p, miR-106a-5p, miR-125a-5p, miR-17-5p, miR-574-3p, miR-339-5p, miR-339-3p, miR-335-5p, miR-16-5p and miR-21-5p.

71. The method of claim 65, wherein the sample comprises blood.

72. The method of claim 71, wherein the blood is venous blood.

73. The method of claim 65, wherein the miRNA expression levels are determined using quantitative real-time PCR (qPCR), sequencing (miRNA-seq), miRNA expression microarrays, DNA biosensors, or any technology that measures RNA.

74. The method of claim 65, wherein the machine-learning classifier is trained with miRNA expression data obtained from a reference population.

75. A classifier for determining the breast cancer status of sample from a subject in need thereof, wherein the classifier identifies the sample as having a specific breast cancer status using as input miRNA expression levels obtained from a miRNA biomarker panel comprises miR-150-5p, miR-106b-3p, miR-106a-5p, miR-125a-5p, miR-17-5p, miR-574-3p, miR-339-5p, miR-339-3p, miR-335-5p, miR-16-5p and miR-21-5p, or a subset thereof from a sample from the subject, and wherein the breast cancer status indicates that the subject can be effectively treated with a breast cancer therapy.

76. The classifier of claim 75, wherein the sample comprises blood.

77. The classifier of claim 76, wherein the blood is venous blood.

78. The classifier of claim 75, wherein the calculation of the breast cancer status comprises obtaining the probability according to a statistical model, wherein the statistical model is a logistic regression.

79. The classifier of claim 78, wherein the statistical model is cross validated with machine learning model.

80. The classifier of claim 79, wherein the machine learning model is Cross Validation Leave One Out.

81. The classifier of claim 80, wherein the calculation of the breast cancer status comprises: (i) averaging the Ct values obtained from the qPCR for each of the miRNAs biomarkers; (ii) subtracting the average value of each of the miRNAs from the Ct value obtained from a control; (iii) squaring the subtracting result of the previous step; (iv) calculating the logarithm in base e for the result of the step iii, obtaining a individual value for each miRNA (VALUE X); (v) calculating the probability by integrating each result of the previous steps according to: p(x)=1/(e\hat{ }(−(β+β*Value X))+1), wherein the β is a specific coefficient related to the statistical model selected; (vi) comparing the obtained probability score (p(x)) with a cut-off point wherein if the value of the individual's probability of having breast cancer is equal to or greater than the cut-off point, the individual will be classified as sick, and if it is less than the cut-off point, it will be classified as healthy.

82. The method of claim 65, wherein the sample is enriched in at least one miRNA from the miRNA biomarker panel comprises miR-150-5p, miR-106b-3p, miR-106a-5p, miR-125a-5p, miR-17-5p, miR-574-3p, miR-339-5p, miR-339-3p, miR-335-5p, miR-16-5p and miR-21-5p.

83. The classifier of claim 75, wherein the sample is enriched in at least one miRNA from the miRNA biomarker panel comprises miR-150-5p, miR-106b-3p, miR-106a-5p, miR-125a-5p, miR-17-5p, miR-574-3p, miR-339-5p, miR-339-3p, miR-335-5p, miR-16-5p and miR-21-5p.

84. A sample comprising body fluid enriched in at least one miRNA from the miRNA biomarker panel comprises miR-150-5p, miR-106b-3p, miR-106a-5p, miR-125a-5p, miR-17-5p, miR-574-3p, miR-339-5p, miR-339-3p, miR-335-5p, miR-16-5p and miR-21-5p.

85. The sample according to claim 84, wherein the body fluid is selected from the consisting of blood, plasma, serum, urine, saliva, lacrimal fluid, and fluids obtainable from the breast glands.

86. The method of claim 65, wherein the breast cancer treatment is based in a breast cancer therapy selected from the group consisting of chemotherapy, anti-hormone therapy, targeted therapy, immunotherapy, and any combination thereof.

87. The classifier of claim 75, wherein the breast cancer treatment is based in a breast cancer therapy selected from the group consisting of chemotherapy, anti-hormone therapy, targeted therapy, immunotherapy, and any combination thereof.

88. The method of claim 65, wherein the breast cancer status comprises absence or presence of breast cancer.

89. The classifier of claim 75, wherein the breast cancer status comprises absence or presence of breast cancer.

90. The method of claim 88, wherein the breast cancer is selected from the group consisting of metastatic, and non-metastatic.

91. The classifier of claim 89, wherein the breast cancer is selected from the group consisting of metastatic, and non-metastatic.

92. The method of claim 65, wherein the breast cancer status comprises a breast cancer risk score.

93. The classifier of claim 75, wherein the breast cancer status comprises a breast cancer risk score.

94. The method of claim 65, wherein the breast cancer status comprises a breast cancer prognosis or outcome score.

95. The classifier of claim 75, wherein the breast cancer status comprises a breast cancer prognosis or outcome score.

96. The method of claim 65, wherein the breast cancer status comprises a breast cancer response to a specific breast cancer therapy.

97. The classifier of claim 75, wherein the breast cancer status comprises a breast cancer response to a specific breast cancer therapy.

98. The method of claim 65, wherein the breast cancer status comprises a breast cancer stage score.

99. The classifier of claim 75, wherein the breast cancer status comprises a breast cancer stage score.

100. The method according to claim 98, wherein the breast cancer stage is selected from the group consisting of: T, N, M and any combination thereof.

101. The classifier according to claim 99, wherein the breast cancer stage is selected from the group consisting of: T, N, M and any combination thereof.

102. The method of claim 66, wherein administering the breast cancer therapy reduces the cancer burden.

103. The method of claim 102, wherein cancer burden is reduced by at least about 10%, at least about 20%, at least about 30%, at least about 40%, or about 50% compared to the cancer burden prior to the administration.

104. The method of claim 103, wherein the subject exhibits progression-free survival of at least about one month, at least about 2 months, at least about 3 months, at least about 4 months, at least about 5 months, at least about 6 months, at least about 7 months, at least about 8 months, at least about 9 months, at least about 10 months, at least about 11 months, at least about one year, at least about eighteen months, at least about two years, at least about three years, at least about four years, or at least about five years after the initial administration.

105. The method of claim 103, wherein the subject exhibits stable disease about one month, about 2 months, about 3 months, about 4 months, about 5 months, about 6 months, about 7 months, about 8 months, about 9 months, about 10 months, about 11 months, about one year, about eighteen months, about two years, about three years, about four years, or about five years after the initial administration.

106. The method of claim 103, wherein the subject exhibits a partial response about one month, about 2 months, about 3 months, about 4 months, about 5 months, about 6 months, about 7 months, about 8 months, about 9 months, about 10 months, about 11 months, about one year, about eighteen months, about two years, about three years, about four years, or about five years after the initial administration.

107. The method of claim 103, wherein the subject exhibits a complete response about one month, about 2 months, about 3 months, about 4 months, about 5 months, about 6 months, about 7 months, about 8 months, about 9 months, about 10 months, about 11 months, about one year, about eighteen months, about two years, about three years, about four years, or about five years after the initial administration.

108. A miRNA biomarker panel comprising miR-150-5p, miR-106b-3p, miR-106a-5p, miR-125a-5p, miR-17-5p, miR-574-3p, miR-339-5p, miR-339-3p, miR-335-5p, miR-16-5p and miR-21-5p, for use in determining the breast cancer status of a subject in need thereof using the classifier according to claim 75, wherein the breast cancer status is used for (i) identifying a subject suitable for an anticancer therapy; (ii) determining the prognosis of a subject undergoing anticancer therapy; (iii) initiating, suspending, or modifying the administration of an anticancer therapy; or, (iv) a combination thereof.

109. The method of claim 65, where the method is implemented in a computer system comprising at least one processor and at least one memory, the at least one memory comprising instructions executed by the at least one processor to cause the at least one processor to implement the machine-learning model.

110. The method of claim 109, further comprising (i) inputting, into the memory of the computer system, the machine-learning model; (ii) inputting, into the memory of the computer system, the miRNA biomarker panel input data corresponding to the subject, wherein the input data comprises miRNA expression levels; (iii) executing the machine-learning model; or (v) any combination thereof.

111. A kit for the detection of breast cancer, comprising: (i) specific oligonucleotides for reverse transcription of miR-16-5p, miR-17-5p, miR-106a-5p, and miR-339-3p in a sample; (ii) oligonucleotides for quantitative PCR of miR-16-5p, miR-17-5p, miR-106a-5p, miR-339-3p; and a universal oligonucleotide Rv.

112. The kit of claim 111, further comprising specific oligonucleotides for the cel39 control.

113. The kit of claim 111, further comprising synthetic positive controls for the quantitative PCR step.


SEQUENCE LISTING (40 sequences)

SEQ ID NO: 1 - RNA, 22 nt, synthetic construct
  source 1..22: mol_type = other RNA; note = hsa-miR-150-5p
tctcccaacccttgtaccagtg

SEQ ID NO: 2 - RNA, 23 nt, synthetic construct
  source 1..23: mol_type = other RNA; note = hsa-miR-17-5p
caaagtgcttacagtgcaggtag

SEQ ID NO: 3 - RNA, 22 nt, synthetic construct
  source 1..22: mol_type = other RNA; note = hsa-miR-574-3p
cacgctcatgcacacacccaca

SEQ ID NO: 4 - RNA, 24 nt, synthetic construct
  source 1..24: mol_type = other RNA; note = hsa-miR-125a-5p
tccctgagaccctttaacctgtga

SEQ ID NO: 5 - RNA, 22 nt, synthetic construct
  source 1..22: mol_type = other RNA; note = hsa-miR-106b-3p
ccgcactgtgggtacttgctgc

SEQ ID NO: 6 - RNA, 22 nt, synthetic construct
  source 1..22: mol_type = other RNA; note = hsa-miR-16-5p
tagcagcacgtaaatattggcg

SEQ ID NO: 7 - RNA, 22 nt, synthetic construct
  source 1..22: mol_type = other RNA; note = hsa-miR-21-5p
tagcttatcagactgatgttga

SEQ ID NO: 8 - RNA, 23 nt, synthetic construct
  source 1..23: mol_type = other RNA; note = hsa-miR-106a-5p
aaaagtgcttacagtgcaggtag

SEQ ID NO: 9 - RNA, 23 nt, synthetic construct
  source 1..23: mol_type = other RNA; note = hsa-miR-339-5p
tccctgtcctccaggagctcacg

SEQ ID NO: 10 - RNA, 23 nt, synthetic construct
  source 1..23: mol_type = other RNA; note = hsa-miR-339-3p
tgagcgcctcgacgacagagccg

SEQ ID NO: 11 - RNA, 23 nt, synthetic construct
  source 1..23: mol_type = other RNA; note = hsa-miR-335-5p
tcaagagcaataacgaaaaatgt

SEQ ID NO: 12 - RNA, 22 nt, synthetic construct
  source 1..22: mol_type = other RNA; note = cel-miR-39-3p
tcaccgggtgtaaatcagcttg

SEQ ID NO: 13 - DNA, 21 bp, synthetic construct
  source 1..21: mol_type = other DNA; note = RT-Stem-loop-Rv
tggtgcagggtccgaggtatt

SEQ ID NO: 14 - DNA, 50 bp, synthetic construct
  source 1..50: mol_type = other DNA; note = RT-hsa-miR-21-5p-STEM
gtctcctctggtgcagggtccgaggtattcgcaccagaggagactcaaca

SEQ ID NO: 15 - DNA, 22 bp, synthetic construct
  source 1..22: mol_type = other DNA; note = RT-hsa-miR-21-5p Fw
cggggggtagcttatcagactg

SEQ ID NO: 16 - DNA, 50 bp, synthetic construct
  source 1..50: mol_type = other DNA; note = RT-hsa-miR-106a-5p-STEM
gtctcctctggtgcagggtccgaggtattcgcaccagaggagacctacct

SEQ ID NO: 17 - DNA, 22 bp, synthetic construct
  source 1..22: mol_type = other DNA; note = RT-hsa-miR-106a-5p Fw
ggcggaaaagtgcttacagtgc

SEQ ID NO: 18 - DNA, 50 bp, synthetic construct
  source 1..50: mol_type = other DNA; note = RT-hsa-miR-125a-5p-STEM
gtctcctctggtgcagggtccgaggtattcgcaccagaggagactcacag

SEQ ID NO: 19 - DNA, 23 bp, synthetic construct
  source 1..23: mol_type = other DNA; note = RT-hsa-miR-125a-5p Fw
ccccctccctgagaccctttaac

SEQ ID NO: 20 - DNA, 50 bp, synthetic construct; identical to SEQ ID NO: 16
  source 1..50: mol_type = other DNA; note = RT-hsa-miR-17-5p-STEM

SEQ ID NO: 21 - DNA, 23 bp, synthetic construct
  source 1..23: mol_type = other DNA; note = RT-hsa-miR-17-5p Fw
ggatggcaaagtgcttacagtgc

SEQ ID NO: 22 - DNA, 50 bp, synthetic construct
  source 1..50: mol_type = other DNA; note = RT-hsa-miR-106b-3p-STEM
gtctcctctggtgcagggtccgaggtattcgcaccagaggagacgcagca

SEQ ID NO: 23 - DNA, 18 bp, synthetic construct
  source 1..18: mol_type = other DNA; note = RT-hsa-miR-106b-3p Fw
gggccgcactgtgggtac

SEQ ID NO: 24 - DNA, 50 bp, synthetic construct
  source 1..50: mol_type = other DNA; note = RT-hsa-miR-150-5p-STEM
gtctcctctggtgcagggtccgaggtattcgcaccagaggagaccactgg

SEQ ID NO: 25 - DNA, 20 bp, synthetic construct
  source 1..20: mol_type = other DNA; note = RT-hsa-miR-150-5p Fw
ccccctctcccaacccttgt

SEQ ID NO: 26 - DNA, 50 bp, synthetic construct
  source 1..50: mol_type = other DNA; note = RT-hsa-miR-16-5p-STEM
gtctcctctggtgcagggtccgaggtattcgcaccagaggagaccgccaa

SEQ ID NO: 27 - DNA, 22 bp, synthetic construct
  source 1..22: mol_type = other DNA; note = RT-hsa-miR-16-5p Fw
ggcccgtagcagcacgtaaata

SEQ ID NO: 28 - DNA, 50 bp, synthetic construct
  source 1..50: mol_type = other DNA; note = RT-hsa-miR-335-5p-STEM
gtctcctctggtgcagggtccgaggtattcgcaccagaggagacacattt

SEQ ID NO: 29 - DNA, 21 bp, synthetic construct
  source 1..21: mol_type = other DNA; note = RT-hsa-miR-335-5p Fw
cggcggtcaagagcaataacg

SEQ ID NO: 30 - DNA, 50 bp, synthetic construct
  source 1..50: mol_type = other DNA; note = RT-hsa-miR-339-3p-STEM
gtctcctctggtgcagggtccgaggtattcgcaccagaggagaccggctc

SEQ ID NO: 31 - DNA, 18 bp, synthetic construct
  source 1..18: mol_type = other DNA; note = RT-hsa-miR-339-3p Fw
ggtgagcgcctcgacgac

SEQ ID NO: 32 - DNA, 50 bp, synthetic construct
  source 1..50: mol_type = other DNA; note = RT-hsa-miR-339-5p-STEM
gtctcctctggtgcagggtccgaggtattcgcaccagaggagaccgtgag

SEQ ID NO: 33 - DNA, 20 bp, synthetic construct
  source 1..20: mol_type = other DNA; note = RT-hsa-miR-339-5p Fw
ccctccctgtcctccaggag

SEQ ID NO: 34 - DNA, 50 bp, synthetic construct
  source 1..50: mol_type = other DNA; note = RT-hsa-miR-574-3p-STEM
gtctcctctggtgcagggtccgaggtattcgcaccagaggagactgtggg

SEQ ID NO: 35 - DNA, 18 bp, synthetic construct
  source 1..18: mol_type = other DNA; note = RT-hsa-miR-574-3p Fw
cgccacgctcatgcacac

SEQ ID NO: 36 - DNA, 50 bp, synthetic construct
  source 1..50: mol_type = other DNA; note = RT-cel-miR-39-3p-STEM
gtctcctctggtgcagggtccgaggtattcgcaccagaggagaccaagct

SEQ ID NO: 37 - DNA, 21 bp, synthetic construct
  source 1..21: mol_type = other DNA; note = RT-cel-miR-39-3p Fw
cggggtcaccgggtgtaaatc

SEQ ID NO: 38 - DNA, 22 bp, synthetic construct
  source 1..22: mol_type = other DNA; note = Primer Universal Rv
tgtgtgcagggtccgaggtatt

SEQ ID NO: 39 - skipped

SEQ ID NO: 40 - DNA, 44 bp, synthetic construct
  source 1..44: mol_type = other DNA; note = Stem-loop sequence
gtctcctctggtgcagggtccgaggtattcgcaccagaggagac